SEQUENCE LISTING (12 sequences)

SEQ ID NO: 1 - DNA, 20 bp, Artificial Sequence
  Oligonucleotide primer
tccatgacgttcctgatgct

SEQ ID NO: 2 - DNA, 20 bp, Artificial Sequence
  Oligonucleotide primer
ttcctgctgtgcttctccac

SEQ ID NO: 3 - DNA, 20 bp, Artificial Sequence
  Oligonucleotide primer
aaggtacctttgcaccctcc

SEQ ID NO: 4 - DNA, 24 bp, Artificial Sequence
  Oligonucleotide primer
tgggacctaaaggtgaagatgctg

SEQ ID NO: 5 - DNA, 22 bp, Artificial Sequence
  Oligonucleotide primer
tgcttgatcactgtgcactggg

SEQ ID NO: 6 - DNA, 26 bp, Artificial Sequence
  Oligonucleotide primer
tcacttcacatggaagctgctatttg

SEQ ID NO: 7 - DNA, 27 bp, Artificial Sequence
  Oligonucleotide primer
ccatggcttgtttataatttcctcctc

SEQ ID NO: 8 - DNA, 22 bp, Artificial Sequence
  Oligonucleotide primer
cgggtctggtttgatgatgtcc

SEQ ID NO: 9 - DNA, 25 bp, Artificial Sequence
  Oligonucleotide primer
gaccctgaccatcactgtcaaagag

SEQ ID NO: 10 - DNA, 24 bp, Artificial Sequence
  Oligonucleotide primer
ggacaggactgaaagacttgctcg

SEQ ID NO: 11 - DNA, 24 bp, Artificial Sequence
  Oligonucleotide primer
tccaacaaagtctggcctgtatcc

SEQ ID NO: 12 - DNA, 31 bp, Artificial Sequence
  Oligonucleotide primer
ttcactgggctgaagcaggtacctaaagggc

(19) United States
(12) Patent Application Publication
(10) Pub. No.: US 2006/0257411 A1
(43) Pub. Date: Nov. 16, 2006

(54) Compositions and methods for modulating cells via CD14 and toll-like receptor 4 signaling pathway

(76) Inventors: Bruce Beutler, San Diego, CA (US); Zhengfan Jiang, San Diego, CA (US)

Correspondence Address: WOODCOCK WASHBURN LLP, ONE LIBERTY PLACE, 46TH FLOOR, 1650 MARKET STREET, PHILADELPHIA, PA, 19103, US

(21) Appl. No.: 11/418,445

(22) Filed: May 4, 2006

Related U.S. Application Data
(60) Provisional application No. 60/678,393, filed on May 6, 2005.

(51) Int. Cl. A61K 39/42; A61K 48/00

(52) U.S. Cl. 424/159.1; 514/44

(57) ABSTRACT

Compositions and methods are provided for screening and identifying compounds which modulate signaling of toll-like receptor 4 (TLR4) pathway via CD14 and a ligand. Methods are provided for treatment of various disease states such as inflammation or autoimmune disease in mammalian subjects by modulating toll-like receptor 4 (TLR4) pathway signaling via CD14 and a ligand. Transgenic non-human animals and methods for developing transgenic non-human animals are provided wherein the transgenic non-human animals comprise a loss-of-function mutation in the CD14 gene.

### CROSS-REFERENCE TO RELATED APPLICATIONS

This application claims the benefit of U.S. Provisional Application No. 60/678,393, filed May 6, 2005, the entire disclosure of which is incorporated herein by reference.

### STATEMENT OF GOVERNMENT SUPPORT

This invention was made by government support by Grant No. U54-AI54523 from National Institutes of Health. The Government has certain rights in this invention.

### FIELD

The present invention relates generally to molecular immunology and the treatment of human diseases. The invention relates to methods for screening and identifying compounds based on the characterization of toll-like receptor 4 (TLR4) pathway signaling via CD14 and a ligand. The invention further provides methods for treatment of various disease states such as infectious disease, inflammation or autoimmune disease in mammalian subjects. The invention further relates to transgenic non-human animals and methods for developing transgenic non-human animals comprising a loss-of-function mutation in the CD14 gene.

### BACKGROUND

Lipopolysaccharide (LPS) is responsible for many of the pathogenic effects of Gram-negative bacteria, but it also induces a protective immune response. O'Brien et al., J. Immunol. 124: 20-24, 1980; Rosenstreich et al., CRC Crit. Rev. Immunol. 3: 263-330, 1982. LPS consists of a lipid A moiety, a core polysaccharide, and an O-polysaccharide of variable length (often more than 50 monosaccharide units). Colony morphology (“smooth” vs. “rough”) is indicative of O-glycosylation status. Microbial variants with long O-polysaccharide chains form smooth colonies; those that lack an O-polysaccharide chain form rough colonies; hence the designations smooth and rough LPS.

Because lipid A, which has no appended sugars at all, is the bioactive moiety of LPS, glycosyl chains are thought to play a subsidiary role in endotoxicity, and there has been no clear evidence that the host distinguishes between smooth and rough LPS chemotypes. Galanos et al., European Journal of Biochemistry 140: 221-227, 1984; Galanos et al., Eur. J. Biochem. 148: 1-5, 1985. Rather, it was supposed that all LPS molecules are engaged by the plasma LPS binding protein (LBP) and transferred to CD 14, a glycosylphosphatidylinisitol (GPI)-anchored protein abundantly expressed on mononuclear phagocytes; events that concentrate the LPS signal. Tobias et al., J. Biol. Chem. 263: 13479-13481, 1988; Tobias et al., J. Biol. Chem. 264: 10867-10871, 1989; Schumann et al., Science 249: 1429-1431, 1990; Wright et al., Science 249: 1431-1433, 1990. All LPS responses are also dependent on the membrane-spanning complex formed by Toll-like receptor 4 (TLR4) and MD-2, through which a signal is propagated. Poltorak et al., Science 282: 2085-2088, 1998; Nagai et al., Nat. Immunol. 3: 667-672, 2002. TLR4 signals by way of four adapter proteins, which appear to operate in functional pairs (MyD88 with Mal (also known as TIRAP), and TRIF with TRAM). Hoebe et al., Nature 424: 743-748, 2003; Yamamoto et al., Science 301: 640-643, 2003; Yamamoto et al., Nat. Immunol. 4: 1144-1150, 2003; Beutler, Nature 430: 257-263, 2004.

The present state of the art indicates that in a mammalian subject with autoimmune disease or infectious disease, the LPS receptor complex utilizes all of these adaptors when activated (MyD88 with Mal, also known as TIRAP; and TRIF with TRAM), and none of them when quiescent. A need exists in the art for improved diagnostic and therapeutic treatment for diseases, for example, autoimmune disease and infectious disease, involving factors that regulate or control the LPS receptor complex of the innate immune system in a mammalian subject.

### SUMMARY

Compositions and methods are provided for screening and identifying compounds which modulate signaling of toll-like receptor 4 (TLR4) pathway via CD14 and a ligand. Methods are provided for treatment of various disease states such as infectious disease, inflammation or autoimmune disease in mammalian subjects by modulating toll-like receptor 4 (TLR4) pathway signaling via CD14 and a ligand. Methods for treating rhabdovirus infection are provided, for example, rabies infection or vesicular stomatitis virus infection, in a mammalian subject methods are provided for screening and identifying compounds for treatment of rhabdovirus infection, e.g., rabies infection or vesicular stomatitis virus infection Transgenic non-human animals and methods for developing transgenic non-human animals are provided wherein the transgenic non-human animals comprise a loss-of-function mutation in the CD14 gene.

A method for treating rhabdovirus infection in a mammalian subject suspected of having an infection is provided which comprises administering to the subject a modulator of Toll-like receptor 4-signaling activity via CD14 in an amount effective to reduce or eliminate the rhabdovirus infection or to prevent its occurrence or recurrence. In one aspect, the modulator is an antagonist of Toll-like receptor 4-signaling activity via CD14. In a further aspect, the modulator is an inhibitor of CD14 activity or Toll-like receptor 4-signaling activity. The inhibitor includes, but is not limited to, interfering RNA, short hairpin RNA, ribozyme, or antisense oligonucleotide to CD14 or TLR-4. In a further aspect, the inhibitor In a further aspect, a monoclonal antibody, a polyclonal antibody, a peptide, peptidomimetic, or a small chemical inhibitor to CD14 or TLR-4. The inhibitor can be, for example, an antibody to CD14 or an antibody to TLR-4. In a detailed aspect, the rhabdovirus is rabies virus or vesicular stomatitis virus.

A method for treating an autoimmune disease in a mammalian subject is provided which comprises administering to the mammalian subject a modulator of Toll-like receptor 4-signaling activity via CD14 in an amount effective to reduce or eliminate the autoimmune disease or to prevent its occurrence or recurrence. In one aspect, the modulator is an antagonist of Toll-like receptor 4-signaling activity via CD14. In a further aspect, the modulator is an inhibitor of CD14 activity or Toll-like receptor 4-signaling activity. The inhibitor includes, but is not limited to, interfering RNA, short hairpin RNA, ribozyme, or antisense oligonucleotide to CD14 or TLR-4. In a further aspect, the inhibitor In a further aspect, a monoclonal antibody, a polyclonal antibody, a peptide, peptidomimetic, or a small chemical inhibitor to CD14 or TLR-4. The inhibitor can be, for example, an antibody to CD14 or an antibody to TLR-4.

A method for treating inflammation in a mammalian subject is provided which comprises administering to the mammalian subject a modulator of Toll-like receptor 4-signaling activity via CD14 in an amount effective to reduce or eliminate inflammation or to prevent its occurrence or recurrence. In one aspect, the modulator is an antagonist of Toll-like receptor 4-signaling activity via CD14. In a further aspect, the modulator is an inhibitor of CD14 activity or Toll-like receptor 4-signaling activity. The inhibitor includes, but is not limited to, interfering RNA, short hairpin RNA, ribozyme, or antisense oligonucleotide to CD14 or TLR-4. In a further aspect, the inhibitor In a further aspect, a monoclonal antibody, a polyclonal antibody, a peptide, peptidomimetic, or a small chemical inhibitor to CD14 or TLR-4. The inhibitor can be, for example, an antibody to CD14 or an antibody to TLR-4.

A method for identifying a compound which modulates signaling in cells via a toll-like receptor 4 pathway is provided which comprises contacting a test compound with a cell-based assay system comprising a cell expressing toll-like receptor 4 capable of signaling responsiveness to a ligand, providing CD14 and the ligand to the assay system in an amount selected to be effective to activate toll-like receptor 4 signaling, and detecting an effect of the test compound on toll-like receptor 4 signaling in the assay system, effectiveness of the test compound in the assay being indicative of the modulation. The method for identifying a compound which modulates signaling in cells via a toll-like receptor 4 pathway further comprises coexpressing CD14 and toll-like receptor 4 in the cell. In a further aspect, the method comprises providing toll-like receptor 4 to the assay system, and detecting an effect of the test compound on CD14/toll-like receptor 4 signaling in the assay system, effectiveness of the test compound in the assay being indicative of the modulation.

In an embodiment of the method, the ligand is an endogenous ligand or an exogenous ligand. The exogenous ligand includes, but is not limited to, lipopolysaccharide, lipid A, di-acylated lipopeptide, tri-acylated lipopeptide, S-MALP-2, R-MALP-2, bacterial lipopeptide, Pam2CSK4, lipoteichoic acid, or zymosan A. In a detailed aspect, the exogenous ligand is rough lipopolysaccharide, smooth lipopolysaccharide, or lipid A from Salmonella minnesota. The endogenous ligand includes, but is not limited to, a lipid. In a further embodiment, the detecting step comprises measuring an effect on tumor necrosis factor production in the cell wherein TNF production is altered in response to rough lipopolysaccharide, but not in response to smooth lipopolysaccharide or lipid A from Salmonella minnesota.

In a further embodiment, the method comprises the detecting step effecting reduced binding of ligand to CD14 by the compound.

In a further embodiment of the method, the detecting step comprises effecting reduced binding of CD14 to toll-like receptor 4 by the compound. In one aspect, the compound is an antagonist of toll-like receptor 4 pathway signaling. In a further aspect, the detecting step comprises measuring a decrease in tumor necrosis factor in the cell assay.

In a further embodiment of the method, the detecting step comprises effecting enhanced binding of ligand to CD14 by the compound.

The method for identifying a compound which modulates signaling in cells via a toll-like receptor 4 pathway further comprises the detecting step which comprises effecting enhanced binding of CD14 to toll-like receptor 4 by the compound. In one aspect, the compound is an agonist of toll-like receptor 4 pathway signaling. In another aspect of the method, the detecting step further comprises measuring an increase in tumor necrosis factor in the cell assay. The cell assay further comprises a macrophage cell.

The method for identifying a compound which modulates signaling in cells via a toll-like receptor 4 pathway further comprises the detecting step which comprises measuring labeled CD14 binding to ligand or labeled CD14 binding to toll-like receptor 4. The label includes, but is not limited to, a radiolabel or a fluorescent label.

In a further embodiment, the method for identifying a compound which modulates signaling in cells via a toll-like receptor 4 pathway is provided wherein the cell expresses TRAM-Trif capable of signaling responsiveness to the ligand, further providing CD14 and the ligand to the assay system in an amount selected to be effective to activate TRAM-Trif signaling, and detecting an effect of the test compound on TRAM-Trif signaling in the assay system, effectiveness of the test compound in the assay being indicative of the modulation.

In one aspect, the method further comprises coexpressing CD14, toll-like receptor 4, and TRAM-Trif in the cell. The method further comprises providing toll-like receptor 4 to the assay system, and detecting an effect of the test compound on CD14/toll-like receptor 4/TRAM-Trif signaling in the assay system, effectiveness of the test compound in the assay being indicative of the modulation. In an aspect of the method, the detecting step further comprises effecting reduced binding of ligand to toll-like receptor 4 by the compound. In an aspect of the method, the detecting step further comprises effecting reduced binding of toll-like receptor 4 to TRAM-Trif by the compound. In an aspect of the method, the detecting step further comprises effecting enhanced binding of ligand to CD14 by the compound. In an aspect of the method, the detecting step further comprises effecting enhanced binding of toll-like receptor 4 to TRAM-Trif by the compound.

In an aspect of the method, the compound is an agonist of TRAM-Trif pathway signaling. In another aspect of the method, the compound is an antagonist of TRAM-Trif pathway signaling. In a further aspect, the ligand is an endogenous ligand or an exogenous ligand. The exogenous ligand includes, but is not limited to, a lipopolysaccharide. The endogenous ligand includes, but is not limited to, a lipid.

In a further aspect of the method, the cell assay comprises a macrophage cell. In a further aspect of the method, the detecting step comprises measuring labeled CD14 binding to ligand or labeled CD14 binding to TLR4 or TRAM-Trif. The label includes, but is not limited to, a radiolabel or fluorescent label.

In a further aspect of the method, the compound is an agonist of TRAM-Trif pathway signaling. The method further comprises the detecting step which comprises measuring an increase in phosphorylation of IRF-3 in the cell assay. In a further aspect, the detecting step comprises measuring an increase in interferon-β in the cell assay. In a further aspect, the detecting step comprises measuring a decreased susceptibility to viral infectivity in the cell assay.

In a further aspect of the method, the compound is an antagonist of TRAM-Trif pathway signaling. The method further comprises the detecting step which comprises measuring a decrease in phosphorylation of IRF-3 in the cell assay. In a further aspect, the detecting step comprises measuring a decrease in interferon-β in the cell assay. In a further aspect, the detecting step comprises measuring an increased susceptibility to viral infectivity in the cell assay.

A transgenic non-human animal is provided comprising a heterologous nucleic acid, wherein the nucleic acid, and the animal exhibits a phenotype, relative to a wild-type phenotype, comprising a characteristic of inhibition of macrophage activation, susceptibility to viral or bacterial infection, a decrease in TNF-α production, or a combination of any two or more thereof. In one aspect, the phenotype of the animal is characteristic of decreased phosphorylation and dimerization of IRF-3 upon induction by lipopolysaccharide, non-responsive INF-β production upon induction by lipopolysaccharide, or macrophage hypersensitivity to cytolysis induced by vesicular stomatitis virus or rabies virus. In a further aspect, the loss-of-function allele in the CD14 gene is a premature stop codon at Q284X. In a detailed aspect, the animal is a mouse or a rat. A cell or cell line can be derived from the transgenic non-human animal comprising the loss-of-function allele of a CD14 gene.

An in vitro method of screening for a modulator of a Toll-like receptor 4- or TRAM-Trif-signaling activity is provided wherein the method comprises contacting a cell or cell line with a test compound wherein the cell or cell line is derived from the transgenic non-human animal, and detecting an increase or a decrease in the amount of TNF-α production, susceptibility to viral or bacterial infection, or a Toll-like receptor 4- or TRAM-Trif-induced macrophage activating activity, thereby identifying the test compound as a modulator of the Toll-like receptor 4- or TRAM-Trif-induced macrophage activating activity.

An in vivo method of screening for a modulator of a Toll-like receptor 4- or TRAM-Trif-signaling activity is provided wherein the method comprises contacting a cell or cell line with a test compound, the cell or cell line derived from the transgenic non-human animal, and detecting an increase or a decrease in the amount of TNF-α production, susceptibility to viral or bacterial infection, or a Toll-like receptor 4- or TRAM-Trif-induced macrophage activating activity, thereby identifying the test compound as a modulator of a Toll-like receptor 4- or TRAM-Trif-induced macrophage activating activity.

A method for screening for a compound which modulates an autoimmune disease is provided comprising contacting a test compound with a cell-based assay system comprising a cell expressing toll-like receptor 4 capable of signaling responsiveness to a ligand, providing CD14 and the ligand to the assay system in an amount selected to be effective to activate toll-like receptor 4 signaling, and detecting an effect of the test compound on toll-like receptor 4 signaling in the assay system, effectiveness of the test compound in the assay being indicative of the modulation of the autoimmune disease. In a further embodiment, the method comprises the cell expressing TRAM-Trif capable of signaling responsiveness to the ligand, providing CD14 and the ligand to the assay system in an amount selected to be effective to activate TRAM-Trif signaling, and detecting an effect of the test compound on TRAM-Trif signaling in the assay system, effectiveness of the test compound in the assay being indicative of the modulation of the autoimmune disease.

In a detailed aspect, the autoimmune disease is insulin-dependent diabetes mellitus, multiple sclerosis, experimental autoimmune encephalomyelitis, rheumatoid arthritis, experimental autoimmune arthritis, myasthenia gravis, thyroiditis, an experimental form of uveoretinitis, Hashimoto's thyroiditis, primary myxoedema, thyrotoxicosis, pernicious anaemia, autoimmune atrophic gastritis, Addison's disease, premature menopause, male infertility, juvenile diabetes, Goodpasture's syndrome, pemphigus vulgaris, pemphigoid, sympathetic ophthalmia, phacogenic uveitis, autoimmune haemolytic anaemia, idiopathic leucopenia, primary biliary cirrhosis, active chronic hepatitis Hbₛ-ve, cryptogenic cirrhosis, ulcerative colitis, Sjogren's syndrome, scleroderma, Wegener's granulomatosis, poly/dermatomyositis, discoid LE or systemic lupus erythematosus.

A method for screening for a compound which modulates an infectious disease is provided which comprises contacting a test compound with a cell-based assay system comprising a cell expressing toll-like receptor 4 capable of signaling responsiveness to a ligand, providing CD14 and the ligand to the assay system in an amount selected to be effective to activate toll-like receptor 4 signaling, and detecting an effect of the test compound on toll-like receptor 4 signaling in the assay system, effectiveness of the test compound in the assay being indicative of the modulation of the infectious disease. In a further embodiment, the method comprises the cell expressing TRAM-Trif capable of signaling responsiveness to the ligand, providing CD14 and the ligand to the assay system in an amount selected to be effective to activate TRAM-Trif signaling, and detecting an effect of the test compound on TRAM-Trif signaling in the assay system, effectiveness of the test compound in the assay being indicative of the modulation of the infectious disease.

The infectious disease can be a bacterial or viral disease. In a detailed aspect, the infectious disease is HIV infection, AIDS, cytomegalovirus infection, or Staphylococcus aureus infection.

### BRIEF DESCRIPTION OF THE DRAWINGS

FIGS. 1a, 1b, 1c, 1d, 1e, 1f, 1g, 1h, 1i, 1j, 1k, and 1l show rough LPS and TLR2-6 specificity of the Heedless mutation.

FIGS. 2a, 2b, 2c, 2d, 2e, 2f, 2g, 2h, 2i, and 2j show Heedless prevents IFN-β induction by LPS.

FIGS. 3a, 3b, 3c, 3d, 3e, and 3f show Heedless macrophages are hypersensitive to cytolysis induced by VSV.

FIG. 4 shows Heedless, a mutation in Cd14, detected by restriction endonuclease cleavage.

FIGS. 5a, 5b, and 5c show rescue of smooth LPS responsiveness in Cd14 homozygous mutant cells by recombinant mCD14.

FIGS. 6a and 6b show the Heedless mutation, mapped and identified by sequencing.

FIG. 7 shows a schematic illustration summarizing the interactions between rough and smooth LPS, the TLR4/MD-2 complex, and CD14.

FIGS. 8a and 8b show a hypothetical mechanism whereby CD14 can permit MyD88-independent signaling from the TLR4 complex.

### DETAILED DESCRIPTION

Compositions and methods are provided for identifying compounds which modulate signaling in cells via a toll-like receptor 4 pathway. CD14 protein plays a role in toll-like receptor signaling and activation via lipopolysaccharide (LPS) sensing. In this study, a mutation in the CD14 gene has advanced the view of LPS sensing, how it occurs, and the limits of specificity of the CD14-MD-2-TLR4 complex. A method for identifying a compound which modulates signaling in cells via a toll-like receptor 4 pathway is provided comprising contacting a test compound with a cell-based assay system comprising a cell expressing toll-like receptor 4 capable of signaling responsiveness to a ligand, providing CD14 and the ligand to the assay system in an amount selected to be effective to activate toll-like receptor 4 signaling, and detecting an effect of the test compound on toll-like receptor 4 signaling in the assay system, effectiveness of the test compound in the assay being indicative of the modulation.

In an effort to identify all proteins responsible for lipopolysaccharide (LPS) sensing and understand their specificities and interactions a program of germline mutagenesis and screening in which macrophages harvested from third generation (G3) mutant C57BL/6 mice are stimulated with diverse TLR activators (including LPS) ex vivo. Tumor necrosis factor (TNF) production is monitored as the primary endpoint of phenotypic analysis. Compositions and methods are provided in which a mutation in the CD14 gene has advanced the view of LPS sensing, how it occurs, and the limits of specificity of the CD14-MD-2-TLR4 complex.

The recessive mutation “Heedless” was detected in third generation (G3) N-ethyl-N-nitrosourea-mutant mice, exhibiting defective responses to microbial inducers. Macrophages from Heedless homozygotes signaled via the MyD88-dependent pathway in response to rough LPS and lipid A, but not in response to smooth LPS. Moreover, the Heedless mutation prevented TRAM-TRIF-dependent signaling in response to all LPS chemotypes. Heedless also abolished macrophage responses to vesicular stomatitis virus (VSV), and substantially inhibited responses to specific ligands for the Toll-like receptor 2 (TLR2)-TLR6 heterodimer. The Heedless phenotype was positionally ascribed to a premature stop codon in Cd14. Ferrero and Goyert, Nucleic Acids Res., 16: 4173, 1988; NCBI GenBank P08571. Our data suggest the TLR4-MD-2 complex distinguishes LPS chemotypes, but CD14 nullifies this distinction. Thus, the TLR4-MD-2 complex receptor can function in two separate modes; one in which full signaling occurs and one limited to MyD88-dependent signaling.

It is to be understood that this invention is not limited to particular methods, reagents, compounds, compositions or biological systems, which can, of course, vary. It is also to be understood that the terminology used herein is for the purpose of describing particular embodiments only, and is not intended to be limiting. As used in this specification and the appended claims, the singular forms “a”, “an” and “the” include plural referents unless the content clearly dictates otherwise. Thus, for example, reference to “a cell” includes a combination of two or more cells, and the like.

“About” as used herein when referring to a measurable value such as an amount, a temporal duration, and the like, is meant to encompass variations of ±20% or ±10%, more preferably ±5%, even more preferably ±1%, and still more preferably +O. 1% from the specified value, as such variations are appropriate to perform the disclosed methods.

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

“Autoimmune disease” refers to a disease caused by an inability of the immune system to distinguish foreign molecules from self molecules, and a loss of immunological tolerance to self antigens, that results in destruction of the self molecules. Autoimmune diseases, include but are not limited to, insulin-dependent diabetes mellitus (IDDM), multiple sclerosis, experimental autoimmune encephalomyelitis (an animal model of multiple sclerosis), rheumatoid arthritis, experimental autoimmune arthritis, myasthenia gravis, thyroiditis, an experimental form of uveoretinitis, Hashimoto's thyroiditis, primary myxoedema, thyrotoxicosis, pernicious anaemia, autoimmune atrophic gastritis, Addison's disease, premature menopause, male infertility, juvenile diabetes, Goodpasture's syndrome, pemphigus vulgaris, pemphigoid, sympathetic ophthalmia, phacogenic uveitis, autoimmune haemolytic anaemia, idiopathic leucopenia, primary biliary cirrhosis, active chronic hepatitis Hbₛ-ve, cryptogenic cirrhosis, ulcerative colitis, Sjogren's syndrome, scleroderma, Wegener's granulomatosis, Poly/Dermatomyositis, discoid LE and systemic Lupus erythematosus.

“Autoantigen” refers to a self-antigen, that is, a substance normally found within a mammal and normally recognized as self, but due to an auto-immune disease, is erroneously recognized as foreign by the mammal. That is, an autoantigen is not recognized as part of the mammal itself by the lymphocytes or antibodies of that mammal and is erroneously attacked by the immunoregulatory system of the mammal as though such autoantigen were a foreign substance. An autoantigen thus acts to downregulate the arm of the immune system that is responsible for causing a specific autoimmune disease. As used herein, “autoantigen” also refers to autoantigenic substances which induce conditions having the symptoms of an autoimmune disease when administered to mammals. An autoantigen according to the invention also includes an epitope or a combination of epitopes derived from an autoantigen that is recognized. As foreign by the mammal and that is a self-antigen in non-disease states.

Autoantigens that are useful according to the invention include but are not limited to those autoantigens associated with suppression of T-cell mediated autoimmune diseases.

An autoantigen refers to a molecule that provokes an immune response, or induces a state of immunological tolerance, including but not limited to single or double stranded DNA, an antibody or fragments thereof, including synthetic peptides of corresponding nucleic acid genetic information, gamma globulins or fragments thereof, including synthetic peptides or corresponding nucleic acid genetic information, a transplantation antigen or fragments thereof, including synthetic peptides or corresponding nucleic acid genetic information. An autoantigen according to the invention also includes an epitope or a combination of epitopes derived from that autoantigen.

“T-cell mediated autoimmune disease” refers to an autoimmune disease wherein the effects of the disease are induced by TH1 mediated stimulation of lymphocyte inflammatory cytokine production. T-cell mediated autoimmune diseases include but are not limited to experimental autoimmune encephalomyelitis, multiple sclerosis, rheumatoid arthritis, myasthenia gravis, thyroiditis, experimental uveoretinitis and adioi disease of the intestine. Autoantigens associated with suppression of TH1 mediated autoimmune diseases include but are not limited to glutamate decarboxylase, insulin, myelin basic protein, type II collagen, nicotinic acetylcholine receptor, thyroglobulin, thyroid peroxidase, and the rhodopsin glycoproteins S-Antigen, IRBP-retinal protein and recoverin.

“Inhibition of macrophage activation” refers to inhibition of TLR4 -induced costimulatory molecule (CD14) expression in macrophages in response to inducers, for example, lipopolysaccharide. CD14 expression on macrophages can be analyzed by FACS.

“Susceptibility to viral or bacterial infection” refers to susceptibility to an infectious virus, e.g., mouse cytomegalovirus (MCMV), or an infectious bacteria, Listeria monocytogenes. Susceptibility to infection with MCMV was measured as the time to death in mice resulting from MCMV infection. Susceptibility to infection with L. monocytogenes was measured as production of TNF and IL-12 p40 mRNA in macrophages of mice infected with L. monocytogenes. Susceptibility to infection with Staphylococcus aureus was measured as the time to death in mice resulting from S. aureus infection.

“Decrease in TNF-α production” refers to macrophages from the mammalian subject that fail to produce normal quantities of TNF-α in response to lipopolysaccharide (a TLR4-selective stimulus).

“Immune cell response” refers to the response of immune system cells to external or internal stimuli (e.g., antigen, cytokines, chemokines, and other cells) producing biochemical changes in the immune cells that result in immune cell migration, killing of target cells, phagocytosis, production of antibodies, other soluble effectors of the immune response, and the like.

“T lymphocyte response” and “T lymphocyte activity” are used here interchangeably to refer to the component of immune response dependent on T lymphocytes (i.e., the proliferation and/or differentiation of T lymphocytes into helper, cytotoxic killer, or suppressor T lymphocytes, the provision of signals by helper T lymphocytes to B lymphocytes that cause or prevent antibody production, the killing of specific target cells by cytotoxic T lymphocytes, and the release of soluble factors such as cytokines that modulate the function of other immune cells).

“Immune response” refers to the concerted action of lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

“Inflammation” or “inflammatory response” refers to an innate immune response that occurs when tissues are injured by bacteria, trauma, toxins, heat, or any other cause. The damaged tissue releases compounds including histamine, bradykinin, and serotonin. Inflammation refers to both acute responses (i.e., responses in which the inflammatory processes are active) and chronic responses (i.e., responses marked by slow progression and formation of new connective tissue). Acute and chronic inflammation can be distinguished by the cell types involved. Acute inflammation often involves polymorphonuclear neutrophils; whereas chronic inflammation is normally characterized by a lymphohistiocytic and/or granulomatous response. Inflammation includes reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory. Such cells include granulocytes, macrophages, neutrophils and eosinophils. Examples of specific types of inflammation are diffuse inflammation, focal inflammation, croupous inflammation, interstitial inflammation, obliterative inflammation, parenchymatous inflammation, reactive inflammation, specific inflammation, toxic inflammation and traumatic inflammation.

“Patient”, “subject” or “mammal” are used interchangeably and refer to mammals such as human patients and non-human primates, as well as experimental animals such as rabbits, rats, and mice, and other animals. Animals include all vertebrates, e.g., mammals and non-mammals, such as sheep, dogs, cows, chickens, amphibians, and reptiles.

“Treating” or “treatment” includes the administration of the compositions, compounds or agents of the present invention to prevent or delay the onset of the symptoms, complications, or biochemical indicia of a disease, alleviating or ameliorating the symptoms or arresting or inhibiting further development of the disease, condition, or disorder (e.g., an infectious disease, inflammation, or an autoimmune disease). “Treating” further refers to any indicia of success in the treatment or amelioration or prevention of the disease, condition, or disorder (e.g., an infectious disease, inflammation, or an autoimmune disease), including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with an infectious disease, inflammation, or an autoimmune disease. The term “therapeutic effect” refers to the reduction, elimination, or prevention of the disease, symptoms of the disease, or side effects of the disease in the subject. “Treating” or “treatment” using the methods of the present invention includes preventing the onset of symptoms in a subject that can be at increased risk of an infectious disease, inflammation, or an autoimmune disease but does not yet experience or exhibit symptoms, inhibiting the symptoms of an infectious disease, inflammation, or an autoimmune disease (slowing or arresting its development), providing relief from the symptoms or side-effects an infectious disease, inflammation, or an autoimmune disease (including palliative treatment), and relieving the symptoms of an infectious disease, inflammation, or an autoimmune disease (causing regression). Treatment can be prophylactic (to prevent or delay the onset of the disease, or to prevent the manifestation of clinical or subclinical symptoms thereof) or therapeutic suppression or alleviation of symptoms after the manifestation of the disease or condition.

A method for treating an infectious disease in a mammalian subject suspected of having an infection is provided comprising administering to the subject a modulator of Toll-like receptor 4-signaling activity via CD14 in an amount effective to reduce or eliminate the rhabdovirus infection or to prevent its occurrence or recurrence. In one aspect, an inhibitor of Toll-like receptor 4-signaling activity via CD14 can be used to treat the infectious viral disease, e.g., rhabdovirus infectious disease. In addition, both gram positive and gram negative bacterial infection and fungal infection can be treated with inhibitors, which would dampen signaling via TLR4 (in the case of gram negative disease) and TLR2 (in the case of gram positive or fungal disease).

A method for treating an autoimmune disease or inflammation in a mammalian subject is provided comprising administering to the mammalian subject a modulator of Toll-like receptor 4-signaling activity via CD14 in an amount effective to reduce or eliminate the autoimmune disease or inflammation or to prevent its occurrence or recurrence. In one aspect, the modulator is an antagonist or inhibitor of Toll-like receptor 4-signaling activity via CD14. Recent studies suggest that hyaluronic acid fragments, produced during inflammation, stimulate TLR4. This suggests that blocking Toll-like receptor 4-signaling activity via CD14 with an antagonist or inhibitor will attenuate inflammation. Jiang, D., et al, Nat Med.; 11: 1173-1179, 2005; Taylor, K R, et al. J Biol Chem. 279: 17079-84, 2004. Termeer, C. et al., J Exp Med. 195: 99-111, 2002

“Inhibitors,” “activators,” and “modulators” of Toll-like receptors in cells are used to refer to inhibitory, activating, or modulating molecules, respectively, identified using in vitro and in vivo assays for Toll-like receptors binding or signaling, e.g., ligands, agonists, antagonists, and their homologs and mimetics.

“Modulator” includes inhibitors and activators. Inhibitors are agents that, e.g., bind to, partially or totally block stimulation, decrease, prevent, delay activation, inactivate, desensitize, or down regulate the activity of Toll-like receptors, e.g., antagonists. Activators are agents that, e.g., bind to, stimulate, increase, open, activate, facilitate, enhance activation, sensitize or up regulate the activity of Toll-like receptors, e.g., agonists. Modulators include agents that, e.g., alter the interaction of Toll-like receptor with: proteins that bind activators or inhibitors, receptors, including proteins, peptides, lipids, carbohydrates, polysaccharides, or combinations of the above, e.g., lipoproteins, glycoproteins, and the like. Modulators include genetically modified versions of naturally-occurring Toll-like receptor ligands, e.g., with altered activity, as well as naturally occurring and synthetic ligands, antagonists, agonists, small chemical molecules and the like. “Cell-based assays” for inhibitors and activators include, e.g., applying putative modulator compounds to a cell expressing a Toll-like receptor and then determining the functional effects on Toll-like receptor signaling, as described herein. “Cell based assays include, but are not limited to, in vivo tissue or cell samples from a mammalian subject or in vitro cell-based assays comprising Toll-like receptor that are treated with a potential activator, inhibitor, or modulator are compared to control samples without the inhibitor, activator, or modulator to examine the extent of inhibition. Control samples (untreated with inhibitors) can be assigned a relative Toll-like receptor activity value of 100%. Inhibition of Toll-like receptor is achieved when the Toll-like receptor activity value relative to the control is about 80%, optionally 50% or 25-0%. Activation of Toll-like receptor is achieved when the Toll-like receptor activity value relative to the control is 110%, optionally 150%, optionally 200-500%, or 1000-3000% higher.

For example, an agonist of Toll-like receptor 4-signaling activity via CD14 may drive signaling that would encourage an adaptive immune response; i.e., useful for vaccination. Also agonists can provide short-term augmentation of host resistance to diverse infections. Having the ability to signal selectively via the MyD88-independent or MyD88-dependent pathways might render special effects, such as lower toxicity and selective induction of type I interferon for MyD88-independent signaling, or selective induction of NF-κB dependent cytokines for MyD88-dependent signaling.

As a further example, an antagonist of Toll-like receptor 4-signaling activity via CD14 may dampen the potentially lethal inflammatory effects of a severe infection, and might be useful in autoimmune disease.

A method for identifying a modulator of signaling in cells via a toll-like receptor 4 pathway is provided which comprises contacting a test compound with a cell-based assay system comprising a cell expressing toll-like receptor 4 capable of signaling responsiveness to a ligand, providing CD14 and the ligand to the assay system in an amount selected to be effective to activate toll-like receptor 4 signaling, and detecting an effect of the test compound on toll-like receptor 4 signaling in the assay system, effectiveness of the test compound in the assay being indicative of the modulation.

In a further aspect, the method provides the cell expressing TRAM-Trif capable of signaling responsiveness to the ligand, providing CD14 and the ligand to the assay system in an amount selected to be effective to activate TRAM-Trif signaling, and detecting an effect of the test compound on TRAM-Trif signaling in the assay system, effectiveness of the test compound in the assay being indicative of the modulation. As described above for TLR4-signaling via CD14, TRAM-TRIF signaling is MyD88-independent signaling. Agonists of TRAM-TRIF signaling selectively leads to interferon production, and less toxicity than activating the receptor in the way that LPS does (stimulating both pathways). Agonists of TRAM-TRIF signaling may be useful in formulating adjuvants and for antiviral effect. Antagonists of TRAM-TRWF signaling may block inflammation to some extent, while partly preserving the induction of IL-6, IL-12, and TNF, which may help to fight infection.

The ability of a molecule to bind to Toll-like receptor can be determined, for example, by the ability of the putative ligand to bind to Toll-like receptor immunoadhesin coated on an assay plate. Specificity of binding can be determined by comparing binding to non-Toll-like receptor.

“Test compound” refers to any compound tested as a modulator of CD14 or toll-like receptor 4. The test compound can be any small organic molecule, or a biological entity, such as a protein, e.g., an antibody or peptide, a sugar, a nucleic acid, e.g., an antisense oligonucleotide, RNAi, or a ribozyme, or a lipid. Alternatively, test compound can be modulators that are genetically altered versions of CD14 protein or toll-like receptor 4 protein. Typically, test compounds will be small organic molecules, peptides, lipids, or lipid analogs.

In one embodiment, antibody binding to Toll-like receptor can be assayed by either immobilizing the ligand or the receptor. For example, the assay can include immobilizing Toll-like receptor fused to a His tag onto Ni-activated NTA resin beads. Antibody can be added in an appropriate buffer and the beads incubated for a period of time at a given temperature. After washes to remove unbound material, the bound protein can be released with, for example, SDS, buffers with a high pH, and the like and analyzed.

“Signaling responsiveness” refers to signaling via a toll-like receptor, e.g., toll-like receptor 4. Signaling responsiveness can refer to, for example, an LPS response dependent on the membrane-spanning complex formed by Toll-like receptor 4 (TLR4) and MD-2, through which a signal is propagated. TLR4 signals by way of four adapter proteins, which appear to operate in functional pairs, MyD88 with Mal (also known as TIRAP), and TRIF with TRAM. Signal generating compounds for measurement in cell-based assays can be genereated, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol.

“Detecting an effect of a test compound on toll-like receptor 4 signaling” can refer to a therapeutic or prophylactic effect in a mammalian subject, such as the reduction, elimination, or prevention of the disease, symptoms of the disease, or side effects of the disease in the subject. “Detecting an effect of a test compound on toll-like receptor 4 signaling” can refer to a compound having an effect in a cell-based assay, e.g., a diagnostic assay, as measured by LPS signaling or lipid A signaling, and measured by TNF-α expression. A loss-of-function mutation in the CD14 gene, e.g., a Heedless mutation, can affect the production of type I IFN. The mutation prevented both smooth LPS and lipid A from signaling via the MyD88-independent pathway. Specifically, CD14 loss-of-function mutation, Heedless, prevented the production of type I IFN and INF-β mRNA, as well as the induction of IFN-inducible genes such as IFIT1, ISG15. In response to lipid A, the formation of the IRF-3 phosphodimer was not detected in heedless mutant cells. Macrophages from CD14 loss-of-function mutation (Heedless) transgenic animals are hypersensitive to cytolysis induced by VSV.

“Concomitant administration” of a known drug with a compound of the present invention means administration of the drug and the compound at such time that both the known drug and the compound will have a therapeutic effect or diagnostic effect. Such concomitant administration can involve concurrent (i.e. at the same time), prior, or subsequent administration of the drug with respect to the administration of a compound of the present invention. A person of ordinary skill in the art, would have no difficulty determining the appropriate timing, sequence and dosages of administration for particular drugs and compounds of the present invention.

In general, the phrase “well tolerated” refers to the absence of adverse changes in health status that occur as a result of the treatment and would affect treatment decisions.

### Antibodies as Modulators of CD14 or Toll-Like Receptor 4

The antibodies and antigen-binding fragments thereof described herein specifically bind to CD14 or to toll-like receptor 4 and can modulate, activate or inhibit an innate immune response to exogenous ligands, for example, rough LPS or lipid A, or in response to vesicular stomatitis virus (VSV) infection or rabies virus infection in a cell, and not in response to the exogenous ligand, smooth LPS.

Antibodies that bind TLR4 or antibodies that bind CD14 are useful as compounds that modulate signaling in celss via a toll-like receptor 4 pathway. See, for example, Akashi, et al., The Journal of Immunology 164: 3471-3475, 2000; Leturcq et al., J Clin Invest. 98: 1533-1538, 1996.

In some embodiments, the antibody or antigen-binding fragment thereof or selectively binds (e.g., competitively binds, or binds to same epitope, e.g., a conformational or a linear epitope) to an antigen that is selectively bound by an antibody produced by a hybridoma cell line. Thus, the epitope can be in close proximity spatially or functionally-associated, e.g., an overlapping or adjacent epitope in linear sequence or conformational space, to a known epitope bound by an antibody. Potential epitopes can be identified computationally using a peptide threading program, and verified using methods known in the art, e.g., by assaying binding of the antibody to mutants or fragments of the toll-like receptor 4 or CD14, e.g., mutants or fragments of a domain of toll-like receptor 4 or CD14.

Methods of determining the sequence of an antibody described herein are known in the art; for example, the sequence of the antibody can be determined by using known techniques to isolate and identify a cDNA encoding the antibody from the hybridoma cell line. Methods for determining the sequence of a cDNA are known in the art.

The antibodies described herein typically have at least one or two heavy chain variable regions (V_{H}), and at least one or two light chain variable regions (V_{L}). The V_{H} and V_{L} regions can be further subdivided into regions of hypervariability, termed complementarity determining regions (CDR), which are interspersed with more highly conserved framework regions (FR). These regions have been precisely defined (see, Kabat et al., Sequences of Proteins of Immunological Interest, Fifth Edition, U.S. Department of Health and Human Services, NIH Publication No. 91-3242, 1991 and Chothia et al., J. Mol. Biol. 196: 901-917, 1987). Antibodies or antibody fragments containing one or more framework regions are also useful in the invention. Such fragments have the ability to specifically bind to a domain of toll-like receptor 4 and to activate or inhibit TNF-α activity in a cell that has been induced by lipopolysaccharide, or to activate or inhibit macrophage responses to vesicular stomatitis virus or rabies virus.

An antibody as described herein can include a heavy and/or light chain constant region (constant regions typically mediate binding between the antibody and host tissues or factors, including effector cells of the immune system and the first component (C1q) of the classical complement system), and can therefore form heavy and light immunoglobulin chains, respectively. For example, the antibody can be a tetramer (two heavy and two light immunoglobulin chains, which can be connected by, for example, disulfide bonds). The antibody can contain only a portion of a heavy chain constant region (e.g., one of the three domains heavy chain domains termed C_{H}1, C_{H}2, and C_{H}3, or a portion of the light chain constant region (e.g., a portion of the region termed C_{L}).

Antigen-binding fragments are also included in the invention. Such fragments can be: (i) a F_{ab} fragment (i.e., a monovalent fragment consisting of the V_{L}, V_{H}, C_{L}, and C_{H}1 domains); (ii) a F(_{ab′})₂ fragment (i.e., a bivalent fragment containing two F_{ab} fragments linked by a disulfide bond at the hinge region); (iii) a F_{d} fragment consisting of the V_{H} and C_{H}1 domains; (iv) a Fᵥ fragment consisting of the V_{L} and V_{H} domains of a single arm of an antibody, (v) a dAb fragment (Ward et al., Nature 341: 544-546, 1989), which consists of a V_{H} domain; and/or (vi) an isolated complementarity determining region (CDR).

Fragments of antibodies (including antigen-binding fragments as described above) can be synthesized using methods known in the art such as in an automated peptide synthesizer, or by expression of a full-length gene or of gene fragments in, for example, E. coli. F(_{ab′})₂ fragments can be produced by pepsin digestion of an antibody molecule, and F_{ab} fragments can be generated by reducing the disulfide bridges of F(_{ab′})₂ fragments. Alternatively, F_{ab} expression libraries can be constructed (Huse et al., Science 246: 1275-81, 1989) to allow relatively rapid identification of monoclonal F_{ab} fragments with the desired specificity.

Methods of making other antibodies and antibody fragments are known in the art. For example, although the two domains of the Fv fragment, V_{L} and V_{H}, are coded for by separate genes, they can be joined, using recombinant methods or a synthetic linker that enables them to be made as a single protein chain in which the V_{L} and V_{H} regions pair to form monovalent molecules (known as single chain Fv (scFv); see e.g., Bird et al., Science 242: 423-426, 1988; Huston et al., Proc. Natl. Acad. Sci. USA 85: 5879-5883, 1988; Colcher et al., Ann. NY Acad. Sci. 880: 263-80, 1999; and Reiter, Clin. Cancer Res. 2: 245-52, 1996).

Techniques for producing single chain antibodies are also described in U.S. Pat. Nos. 4,946,778 and 4,704,692. Such single chain antibodies are encompassed within the term “antigen-binding fragment” of an antibody. These antibody fragments are obtained using conventional techniques known to those of ordinary skill in the art, and the fragments are screened for utility in the same manner that intact antibodies are screened. Moreover, a single chain antibody can form complexes or multimers and, thereby, become a multivalent antibody having specificities for different epitopes of the same target protein.

Antibodies and portions thereof that are described herein can be monoclonal antibodies, generated from monoclonal antibodies, or can be produced by synthetic methods known in the art. Antibodies can be recombinantly produced (e.g., produced by phage display or by combinatorial methods, as described in, e.g., U.S. Pat. No. 5,223,409; WO 92/18619; WO 91/17271; WO 92/20791; WO 92/15679; WO 93/01288; WO 92/01047; WO 92/09690; WO 90/02809; Fuchs et al., Bio/Technology 9: 1370-1372, 1991; Hay et al., Human Antibody Hybridomas 3: 81-85, 1992; Huse et al., Science 246: 1275-1281, 1989; Griffiths et al., EMBO J. 12: 725-734, 1993; Hawkins et al., J. Mol. Biol. 226: 889-896, 1992; Clackson et al., Nature 352: 624-628, 1991; Gram et al., Proc. Natl. Acad. Sci. USA 89: 3576-3580, 1992; Garrad et al., Bio/Technology 9: 1373-1377, 1991; Hoogenboom et al., Nucl. Acids Res. 19: 4133-4137, 1991; and Barbas et al., Proc. Natl. Acad. Sci. USA 88: 7978-7982, 1991).

As one example, a toll-like receptor 4 antibody or CD14 antibody can be made by immunizing an animal with a TLR4 polypeptide or CD14 polypeptide, or fragment (e.g., an antigenic peptide fragment derived from (i.e., having the sequence of a portion of) TLR4 or CD14 thereof, or a cell expressing the TLR4 antigen or CD14 antigen or an antigenic fragment thereof. In some embodiments, antibodies or antigen-binding fragments thereof described herein can bind to a purified TLR4 or CD14. In some embodiments, the antibodies or antigen-binding fragments thereof can bind to a TLR4 or CD14 in a tissue section, a whole cell (living, lysed, or fractionated), or a membrane fraction. Antibodies can be tested, e.g., in in vitro systems such as peripheral blood mononuclear cells (PBMCs), for the ability to activate or inhibit TNF-α activity in a cell that has been induced by lipopolysaccharide, or to activate or inhibit macrophage response to vesicular stomatitis virus or rabies virus.

In the event an antigenic peptide derived from TLR4 or CD14 is used, it will typically include at least eight (e.g., 10, 15, 20, 30, 50, 100 or more) consecutive amino acid residues of a domain of TLR4 or CD14. In some embodiments, the antigenic peptide will comprise all of the domain of TLR4 or CD14. The antibodies generated can specifically bind to one of the proteins in their native form (thus, antibodies with linear or conformational epitopes are within the invention), in a denatured or otherwise non-native form, or both. Peptides likely to be antigenic can be identified by methods known in the art, e.g., by computer-based antigenicity-predicting algorithms. Conformational epitopes can sometimes be identified by identifying antibodies that bind to a protein in its native form, but not in a denatured form.

The host animal (e.g., a rabbit, mouse, guinea pig, or rat) can be immunized with the antigen, optionally linked to a carrier (i.e., a substance that stabilizes or otherwise improves the immunogenicity of an associated molecule), and optionally administered with an adjuvant (see, e.g., Ausubel et al., supra). An exemplary carrier is keyhole limpet hemocyanin (KLH) and exemplary adjuvants, which will typically be selected in view of the host animal's species, include Freund's adjuvant (complete or incomplete), adjuvant mineral gels (e.g., aluminum hydroxide), surface active substances such as lysolecithin, pluronic polyols, polyanions, peptides, oil emulsions, dinitrophenol, BCG (bacille Calmette-Guerin), and Corynebacterium parvum. KLH is also sometimes referred to as an adjuvant. The antibodies generated in the host can be purified by, for example, affinity chromatography methods in which the polypeptide antigen or a fragment thereof, is immobilized on a resin.

Epitopes encompassed by an antigenic peptide will typically be located on the surface of the protein (e.g., in hydrophilic regions), or in regions that are highly antigenic (such regions can be selected, initially, by virtue of containing many charged residues). An Emini surface probability analysis of human protein sequences can be used to indicate the regions that have a particularly high probability of being localized to the surface of the protein.

The antibody can be a fully human antibody (e.g., an antibody made in a mouse or other mammal that has been genetically engineered to produce an antibody from a human immunoglobulin sequence, such as that of a human immunoglobulin gene (the kappa, lambda, alpha (IgA₁ and IgA₂), gamma (IgG₁, IgG₂, IgG₃, IgG₄), delta, epsilon and mu constant region genes or the myriad immunoglobulin variable region genes). Alternatively, the antibody can be a non-human antibody (e.g., a rodent (e.g., a mouse or rat), goat, rabbit, or non-human primate (e.g., monkey) antibody).

Human monoclonal antibodies can be generated in transgenic mice carrying the human immunoglobulin genes rather than those of the mouse. Splenocytes obtained from these mice (after immunization with an antigen of interest) can be used to produce hybridomas that secrete human mAbs with specific affinities for epitopes from a human protein (see, e.g., WO 91/00906, WO 91/10741; WO 92/03918; WO 92/03917; Lonberg et al., Nature 368: 856-859, 1994; Green et al., Nature Genet. 7: 13-21, 1994; Morrison et al., Proc. Natl. Acad. Sci. USA 81: 6851-6855, 1994; Bruggeman et al., Immunol. 7: 33-40, 1993; Tuaillon et al., Proc. Natl. Acad. Sci. USA 90: 3720-3724, 1993; and Bruggeman et al., Eur. J. Immunol. 21: 1323-1326, 1991).

The anti-TLR4 antibody or anti-CD14 antibody can also be one in which the variable region, or a portion thereof (e.g., a CDR), is generated in a non-human organism (e.g., a rat or mouse). Thus, the invention encompasses chimeric, CDR-grafted, and humanized antibodies and antibodies that are generated in a non-human organism and then modified (in, e.g., the variable framework or constant region) to decrease antigenicity in a human. Chimeric antibodies (i.e., antibodies in which different portions are derived from different animal species (e.g., the variable region of a murine mAb and the constant region of a human immunoglobulin) can be produced by recombinant techniques known in the art. For example, a gene encoding the F_{c} constant region of a murine (or other species) monoclonal antibody molecule can be digested with restriction enzymes to remove the region encoding the murine F_{c}, and the equivalent portion of a gene encoding a human F_{c} constant region can be substituted therefore (see, e.g., European Patent Application Nos. 125,023; 184,187; 171,496; and 173,494; see also WO 86/01533; U.S. Pat. No. 4,816,567; Better et al., Science 240: 1041-1043, 1988; Liu et al., Proc. Natl. Acad. Sci. USA 84: 3439-3443, 1987; Liu et al., J. Immunol. 139: 3521-3526, 1987; Sun et al., Proc. Natl. Acad. Sci. USA 84: 214-218, 1987; Nishimura et al., Cancer Res. 47: 999-1005, 1987; Wood et al., Nature 314: 446-449, 1985; Shaw et al., J. Natl. Cancer Inst. 80: 1553-1559, 1988; Morrison et al., Proc. Natl. Acad. Sci. USA 81: 6851, 1984; Neuberger et al., Nature 312: 604, 1984; and Takeda et al., Nature 314: 452, 1984).

In a humanized or CDR-grafted antibody, at least one or two, but generally all three of the recipient CDRs (of heavy and or light immunoglobulin chains) will be replaced with a donor CDR (see, e.g., U.S. Pat. No. 5,225,539; Jones et al., Nature 321: 552-525, 1986; Verhoeyan et al., Science 239: 1534, 1988; and Beidler et al., J. Immunol. 141: 4053-4060, 1988). One need replace only the number of CDRs required for binding of the humanized antibody to toll-like receptor 4or CD14. The donor can be a rodent antibody, and the recipient can be a human framework or a human consensus framework. Typically, the immunoglobulin providing the CDRs is called the “donor” (and is often that of a rodent) and the immunoglobulin providing the framework is called the “acceptor.” The acceptor framework can be a naturally occurring (e.g., a human) framework, a consensus framework or sequence, or a sequence that is at least 85% (e.g., 90%, 95%, 99%) identical thereto. A “consensus sequence” is one formed from the most frequently occurring amino acids (or nucleotides) in a family of related sequences (see, e.g., Winnaker, From Genes to Clones, Verlagsgesellschaft, Weinheim, Germany, 1987). Each position in the consensus sequence is occupied by the amino acid residue that occurs most frequently at that position in the family (where two occur equally frequently, either can be included). A “consensus framework” refers to the framework region in the consensus immunoglobulin sequence. Humanized antibodies to toll-like receptor 4 or CD14 can be made in which specific amino acid residues have been substituted, deleted or added (in, e.g., in the framework region to improve antigen binding). For example, a humanized antibody will have framework residues identical to those of the donor or to amino acid a receptor other than those of the recipient framework residue. To generate such antibodies, a selected, small number of acceptor framework residues of the humanized immunoglobulin chain are replaced by the corresponding donor amino acids. The substitutions can occur adjacent to the CDR or in regions that interact with a CDR (U.S. Pat. No. 5,585,089, see especially columns 12-16). Other techniques for humanizing antibodies are described in EP 519596 A1.

A toll-like receptor 4 antibody or CD14 antibody can be humanized as described above or using other methods known in the art. For example, humanized antibodies can be generated by replacing sequences of the Fv variable region that are not directly involved in antigen binding with equivalent sequences from human Fv variable regions. General methods for generating humanized antibodies are provided by Morrison, Science 229: 1202-1207, 1985; Oi et al., BioTechniques 4: 214, 1986, and Queen et al. (U.S. Pat. Nos. 5,585,089; 5,693,761, and 5,693,762). The nucleic acid sequences required by these methods can be obtained from a hybridoma producing an antibody against toll-like receptor 4 or CD14 or fragments thereof having the desired properties such as the ability to activate or inhibit TNF-α activity in a cell that has been induced by lipopolysaccharide, or to activate or inhibit macrophage response to vesicular stomatitis virus or rabies virus. The recombinant DNA encoding the humanized antibody, or fragment thereof, can then be cloned into an appropriate expression vector.

In certain embodiments, the antibody has an effector function and can fix complement, while in others it can neither recruit effector cells nor fix complement. The antibody can also have little or no ability to bind an Fc receptor. For example, it can be an isotype or subtype, or a fragment or other mutant that cannot bind to an Fc receptor (e.g., the antibody can have a mutant (e.g., a deleted) Fc receptor binding region). Antibodies lacking the Fc region typically cannot fix complement, and thus are less likely to cause the death of the cells they bind to.

In other embodiments, the antibody can be coupled to a heterologous substance, such as a therapeutic agent (e.g., an antibiotic), or a detectable label. A detectable label can include an enzyme (e.g., horseradish peroxidase, alkaline phosphatase, .beta.-galactosidase, or acetylcholinesterase), a prosthetic group (e.g., streptavidin/biotin and avidin/biotin), or a fluorescent, luminescent, bioluminescent, or radioactive material (e.g., umbelliferone, fluorescein, fluorescein isothiocyanate, rhodamine, dichlorotriazinylamine fluorescein, dansyl chloride or phycoerythrin (which are fluorescent), luminol (which is luminescent), luciferase, luciferin, and aequorin (which are bioluminescent), and ⁹⁹mTc, ¹⁸⁸Re, ¹¹¹In, ¹²⁵I, ¹³¹I, ³⁵S or ³H (which are radioactive)).

The antibodies described herein (e.g., monoclonal antibodies) can also be used to isolate toll-like receptor 4 or CD14 proteins or fragments thereof such as the fragment associated with activation or inhibition of TNF-α activity in a cell that has been induced by lipopolysaccharide, or to activation or inhibition of macrophage response to vesicular stomatitis virus or rabies virus (by, for example, affinity chromatography or immunoprecipitation) or to detect them in, for example, a cell lysate or supernatant (by Western blotting, enzyme-linked immunosorbant assays (ELISAs), radioimmune assays, and the like) or a histological section. These methods permit the determination of the abundance and pattern of expression of a particular protein. This information can be useful in making a diagnosis or in evaluating the efficacy of a clinical test or treatment.

The invention also includes the nucleic acids that encode the antibodies described above and vectors and cells (e.g., mammalian cells such as CHO cells or lymphatic cells) that contain them (e.g., cells transformed with a nucleic acid that encodes an antibody that specifically binds to toll-like receptor 4 or CD14). Similarly, the invention includes cell lines (e.g., hybridomas) that make the antibodies of the invention and methods of making those cell lines.

### Immunofogical Detection of CD14 or Toll-Like Receptor 4 Polypeptides and Modulators Thereof

In addition to the detection of CD14 gene or toll-like receptor 4 gene and gene expression using nucleic acid hybridization technology, one can also use immunoassays to detect CD14 or toll-like receptor 4 proteins. Such assays are useful for screening for modulators of CD14 or toll-like receptor 4, as well as for therapeutic and diagnostic applications. Imrunoassays can be used to qualitatively or quantitatively analyze CD14 protein or toll-like receptor 4 protein. A general overview of the applicable technology can be found in Harlow & Lane, Antibodies: A Laboratory Manual, 1988.

A. Production of Antibodies

Methods of producing polyclonal and monoclonal antibodies that react specifically with CD14 or toll-like receptor 4 proteins are known to those of skill in the art (see, e.g., Coligan, Current Protocols in Immunology, 1991; Harlow & Lane, supra; Goding, Monoclonal Antibodies: Principles and Practice, 2d ed. 1986; and Kohler et al., Nature 256: 495-497, 1975. Such techniques include antibody preparation by selection of antibodies from libraries of recombinant antibodies in phage or similar vectors, as well as preparation of polyclonal and monoclonal antibodies by immunizing rabbits or mice (see, e.g., Huse et al., Science 246: 1275-1281, 1989; Ward et al., Nature 341: 544-546, 1989).

A number of immunogens comprising portions of CD14 protein or toll-like receptor 4 protein can be used to produce antibodies specifically reactive with CD14 protein or toll-like receptor 4 protein. For example, recombinant CD14 protein or toll-like receptor 4 protein or an antigenic fragment thereof, can be isolated as described herein. Recombinant protein can be expressed in eukaryotic or prokaryotic cells as described above, and purified as generally described above. Recombinant protein is the preferred immunogen for the production of monoclonal or polyclonal antibodies. Alternatively, a synthetic peptide derived from the sequences disclosed herein and conjugated to a carrier protein can be used an immunogen. Naturally occurring protein canalso be used either in pure or impure form. The product is then injected into an animal capable of producing antibodies. Either monoclonal or polyclonal antibodies can be generated, for subsequent use in immunoassays to measure the protein.

Methods of production of polyclonal antibodies are known to those of skill in the art. An inbred strain of mice (e.g., BALB/C mice) or rabbits is immunized with the protein using a standard adjuvant, such as Freund's adjuvant, and a standard immunization protocol. The animal's immune response to the immunogen preparation is monitored by taking test bleeds and determining the titer of reactivity to the beta subunits. When appropriately high titers of antibody to the immunogen are obtained, blood is collected from the animal and antisera are prepared. Further fractionation of the antisera to enrich for antibodies reactive to the protein can be done if desired (see, Harlow & Lane, supra).

Monoclonal antibodies can be obtained by various techniques familiar to those skilled in the art. Briefly, spleen cells from an animal immunized with a desired antigen are immortalized, commonly by fusion with a myeloma cell (see, Kohler et al., Eur. J. Immunol. 6: 511-519, 1976). Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells can be enhanced by various techniques, including injection into the peritoneal cavity of a vertebrate host. Alternatively, one canisolate DNA sequences which encode a monoclonal antibody or a binding fragment thereof by screening a DNA library from human B cells according to the general protocol outlined by Huse, et al., Science 246: 1275-1281, 1989.

Monoclonal antibodies and polyclonal sera are collected and titered against the immunogen protein in an immunoassay, for example, a solid phase immunoassay with the immunogen immobilized on a solid support. Typically, polyclonal antisera with a titer of 10⁴ or greater are selected and tested for their cross reactivity against non-CD14 or toll-like receptor 4 proteins, using a competitive binding immunoassay. Specific polyclonal antisera and monoclonal antibodies will usually bind with a K_{d} of at least about 0.1 mM, more usually at least about 1 μM, preferably at least about 0.1 μM or better, and most preferably, 0.01 μM or better. Antibodies specific only for a particular CD14 ortholog or toll-like receptor 4 ortholog, such as human CD14 or human toll-like receptor 4, can also be made, by subtracting out other cross-reacting orthologs from a species such as a non-human mammal. In this manner, antibodies that bind only to CD14 or toll-like receptor 4 can be obtained.

Once the specific antibodies against CD14 protein or toll-like receptor 4 protein are available, the protein can be detected by a variety of immunoassay methods. In addition, the antibody can be used therapeutically as modulators of CD14 or toll-like receptor 4. For a review of immunological and immunoassay procedures, see Basic and Clinical Immunology (Stites & Terr eds., 7^{th} ed. 1991). Moreover, the immunoassays of the present invention can be performed in any of several configurations, which are reviewed extensively in Enzyme Immunoassay (Maggio, ed., 1980); and Harlow & Lane, supra.

B. Immunological Binding Assays

CD14 protein or toll-like receptor 4 protein can be detected and/or quantified using any of a number of well recognized immunological binding assays (see, e.g., U.S. Pat. Nos. 4,366,241; 4,376,110; 4,517,288; and 4,837,168). For a review of the general immunoassays, see also Methods in Cell Biology: Antibodies in Cell Biology, volume 37 (Asai, ed. 1993); Basic and Clinical Immunology (Stites & Terr, eds., 7th ed. 1991). Immunological binding assays (or immunoassays) typically use an antibody that specifically binds to a protein or antigen of choice (in this case CD14 protein or toll-like receptor 4 protein or antigenic subsequence thereof). The antibody (e.g., anti-CD14 or anti-toll-like receptor 4) can be produced by any of a number of means well known to those of skill in the art and as described above.

Immunoassays also often use a labeling agent to specifically bind to and label the complex formed by the antibody and antigen. The labeling agent can itself be one of the moieties comprising the antibody/antigen complex. Thus, the labeling agent can be a labeled CD14 or labeled toll-like receptor 4 or a labeled anti-CD14 or anti-toll-like receptor 4 antibody. Alternatively, the labeling agent can be a third moiety, such a secondary antibody, that specifically binds to the antibody/CD14 or antibody/toll-like receptor 4 complex (a secondary antibody is typically specific to antibodies of the species from which the first antibody is derived). Other proteins capable of specifically binding immunoglobulin constant regions, such as protein A or protein G can also be used as the label agent. These proteins exhibit a strong non-immunogenic reactivity with immunoglobulin constant regions from a variety of species (see, e.g., Kronval et al., J. Immunol. 111: 1401-1406, 1973; Akerstrom et al., J. Immunol. 135: 2589-2542, 1985). The labeling agent can be modified with a detectable moiety, such as biotin, to which another molecule can specifically bind, such as streptavidin. A variety of detectable moieties are well known to those skilled in the art.

Throughout the assays, incubation and/or washing steps can be required after each combination of reagents. Incubation steps can vary from about 5 seconds to several hours, optionally from about 5 minutes to about 24 hours. However, the incubation time will depend upon the assay format, antigen, volume of solution, concentrations, and the like. Usually, the assays will be carried out at ambient temperature, although they can be conducted over a range of temperatures, such as 10° C. to 40° C.

Non-competitive assay formats: Immunoassays for detecting CD14 or toll-like receptor 4 in samples can be either competitive or noncompetitive. Noncompetitive immunoassays are assays in which the amount of antigen is directly measured. In one preferred “sandwich” assay, for example, the anti-CD14 or anti-toll-like receptor 4 antibodies can be bound directly to a solid substrate on which they are immobilized. These immobilized antibodies then capture CD14 or toll-like receptor 4 present in the test sample. CD14 protein or toll-like receptor 4 protein thus immobilized are then bound by a labeling agent, such as a second CD14 antibody or toll-like receptor 4 antibody bearing a label. Alternatively, the second antibody can lack a label, but it can, in turn, be bound by a labeled third antibody specific to antibodies of the species from which the second antibody is derived. The second or third antibody is typically modified with a detectable moiety, such as biotin, to which another molecule specifically binds, e.g., streptavidin, to provide a detectable moiety.

Competitive assay formats: In competitive assays, the amount of CD14 protein or toll-like receptor 4 protein present in the sample is measured indirectly by measuring the amount of a known, added (exogenous) CD14 protein or toll-like receptor 4 protein displaced (competed away) from an anti-CD14 or anti-toll-like receptor 4 antibody by the unknown CD14 protein or toll-like receptor 4 protein present in a sample. In one competitive assay, a known amount of CD14 protein or toll-like receptor 4 protein is added to a sample and the sample is then contacted with an antibody that specifically binds to CD14 protein or toll-like receptor 4 protein. The amount of exogenous CD14 protein or toll-like receptor 4 protein bound to the antibody is inversely proportional to the concentration of CD14 protein or toll-like receptor 4 protein present in the sample. In a particularly preferred embodiment, the antibody is immobilized on a solid substrate. The amount of CD14 protein or toll-like receptor 4 protein bound to the antibody can be determined either by measuring the amount of CD14 or toll-like receptor 4 present in CD14 protein/antibody complex or toll-like receptor 4 protein/antibody complex, or alternatively by measuring the amount of remaining uncomplexed protein. The amount of CD14 protein or toll-like receptor 4 protein can be detected by providing a labeled CD14 molecule or toll-like receptor 4 molecule.

A hapten inhibition assay is another preferred competitive assay. In this assay the known CD14 protein or toll-like receptor 4 protein is immobilized on a solid substrate. A known amount of anti-CD14 antibody or anti-toll-like receptor 4 antibody is added to the sample, and the sample is then contacted with the immobilized CD14 or toll-like receptor 4. The amount of anti-CD14 antibody or anti-toll-like receptor 4 antibody bound to the known immobilized CD14 or toll-like receptor 4 is inversely proportional to the amount of CD14 protein or toll-like receptor 4 protein present in the sample. Again, the amount of immobilized antibody can be detected by detecting either the immobilized fraction of antibody or the fraction of the antibody that remains in solution. Detection can be direct where the antibody is labeled or indirect by the subsequent addition of a labeled moiety that specifically binds to the antibody as described above.

Cross-reactivity determinations: Immunoassays in the competitive binding format can also be used for crossreactivity determinations. For example, CD14 protein or toll-like receptor 4 protein can be immobilized to a solid support. Proteins (e.g., CD14 or toll-like receptor 4 and homologs) are added to the assay that compete for binding of the antisera to the immobilized antigen. The ability of the added proteins to compete for binding of the antisera to the immobilized protein is compared to the ability of CD14 protein or toll-like receptor 4 protein to compete with itself. The percent crossreactivity for the above proteins is calculated, using standard calculations. Those antisera with less than 10% crossreactivity with each of the added proteins listed above are selected and pooled. The cross-reacting antibodies are optionally removed from the pooled antisera by immunoabsorption with the added considered proteins, e.g., distantly related homologs.

The immunoabsorbed and pooled antisera are then used in a competitive binding immunoassay as described above to compare a second protein, thought to be perhaps an allele or polymorphic variant of CD14 protein or toll-like receptor 4 protein, to the immunogen protein. In order to make this comparison, the two proteins are each assayed at a wide range of concentrations and the amount of each protein required to inhibit 50% of the binding of the antisera to the immobilized protein is determined. If the amount of the second protein required to inhibit 50% of binding is less than 10 times the amount of CD14 protein or toll-like receptor 4 protein that is required to inhibit 50% of binding, then the second protein is said to specifically bind to the polyclonal antibodies generated to CD14 or toll-like receptor 4 immunogen.

Other assay formats: Western blot (immunoblot) analysis is used to detect and quantify the presence of CD14 protein or toll-like receptor 4 protein in the sample. The technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind CD14 protein or toll-like receptor 4 protein. The anti-CD14 antibody or anti-toll-like receptor 4 antibody specifically bind to CD14 or toll-like receptor 4 on the solid support. These antibodies can be directly labeled or alternatively can be subsequently detected using labeled antibodies (e.g., labeled sheep anti-mouse antibodies) that specifically bind to the anti-CD14 antibody or anti-toll-like receptor 4 antibody.

Other assay formats include liposome immunoassays (LIA), which use liposomes designed to bind specific molecules (e.g., antibodies) and release encapsulated reagents or markers. The released chemicals are then detected according to standard techniques (see Monroe et al., Amer. Clin. Prod. Rev. 5: 34-41, 1986).

Reduction of non-specific binding: One of skill in the art will appreciate that it is often desirable to minimize non-specific binding in immunoassays. Particularly, where the assay involves an antigen or antibody immobilized on a solid substrate it is desirable to minimize the amount of non-specific binding to the substrate. Means of reducing such non-specific binding are well known to those of skill in the art. Typically, this technique involves coating the substrate with a proteinaceous composition. In particular, protein compositions such as bovine serum albumin (BSA), nonfat powdered milk, and gelatin are widely used with powdered milk being most preferred.

Labels: The particular label or detectable group used in the assay is not a critical aspect of the invention, as long as it does not significantly interfere with the specific binding of the antibody used in the assay. The detectable group can be any material having a detectable physical or chemical property. Such detectable labels have been well-developed in the field of immunoassays and, in general, most any label useful in such methods can be applied to the present invention. Thus, a label is any composition detectable by spectroscopic, photochemical, biochemical, immunochemical, electrical, optical or chemical means. Useful labels in the present invention include magnetic beads (e.g., DYNABEADS™), fluorescent dyes (e.g., fluorescein isothiocyanate, Texas red, rhodamine, and the like), radiolabels (e.g., ³H, ¹²⁵I, ³⁵S, ¹⁴C, or ³²P), enzymes (e.g., horse radish peroxidase, alkaline phosphatase and others commonly used in an ELISA), chemiluminescent labels, and colorimetric labels such as colloidal gold or colored glass or plastic beads (e.g., polystyrene, polypropylene, latex, etc.).

The label can be coupled directly or indirectly to the desired component of the assay according to methods well known in the art. As indicated above, a wide variety of labels can be used, with the choice of label depending on sensitivity required, ease of conjugation with the compound, stability requirements, available instrumentation, and disposal provisions.

Non-radioactive labels are often attached by indirect means. Generally, a ligand molecule (e.g., biotin) is covalently bound to the molecule. The ligand then binds to another molecules (e.g., streptavidin) molecule, which is either inherently detectable or covalently bound to a signal system, such as a detectable enzyme, a fluorescent compound, or a chemiluminescent compound. The ligands and their targets can be used in any suitable combination with antibodies that recognize CD14 protein or toll-like receptor 4 protein, or secondary antibodies that recognize anti-CD14 antibody or anti-toll-like receptor 4 antibody.

The molecules can also be conjugated directly to signal generating compounds, e.g., by conjugation with an enzyme or fluorophore. Enzymes of interest as labels will primarily be hydrolases, particularly phosphatases, esterases and glycosidases, or oxidotases, particularly peroxidases. Fluorescent compounds include fluorescein and its derivatives, rhodamine and its derivatives, dansyl, umbelliferone, etc. Chemiluminescent compounds include luciferin, and 2,3-dihydrophthalazinediones, e.g., luminol. For a review of various labeling or signal producing systems that can be used, see U.S. Pat. No. 4,391,904.

Means of detecting labels are well known to those of skill in the art. Thus, for example, where the label is a radioactive label, means for detection include a scintillation counter or photographic film as in autoradiography. Where the label is a fluorescent label, it can be detected by exciting the fluorochrome with the appropriate wavelength of light and detecting the resulting fluorescence. The fluorescence can be detected visually, by the use of electronic detectors such as charge coupled devices (CCDs) or photomultipliers and the like. Similarly, enzymatic labels can be detected by providing the appropriate substrates for the enzyme and detecting the resulting reaction product. Finally simple colorimetric labels can be detected simply by observing the color associated with the label. Thus, in various dipstick assays, conjugated gold often appears pink, while various conjugated beads appear the color of the bead.

Some assay formats do not require the use of labeled components. For instance, agglutination assays can be used to detect the presence of the target antibodies. In this case, antigen-coated particles are agglutinated by samples comprising the target antibodies. In this format, none of the components need be labeled and the presence of the target antibody is detected by simple visual inspection.

### High Throughput Assays for Modulators of CD14 or Toll-Like Receptor 4

The compounds tested as modulators of CD14 or toll-like receptor 4 can be any small organic molecule, or a biological entity, such as a protein, e.g., an antibody or peptide, a sugar, a nucleic acid, e.g., an antisense oligonucleotide, RNAi, or a ribozyme, or a lipid. Alternatively, modulators can be genetically altered versions of CD14 protein or toll-like receptor 4 protein. Typically, test compounds will be small organic molecules, peptides, lipids, and lipid analogs.

Essentially any chemical compound can be used as a potential modulator or ligand in the assays of the invention, although most often compounds can be dissolved in aqueous or organic (especially DMSO-based) solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

In one preferred embodiment, high throughput screening methods involve providing a combinatorial small organic molecule or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37: 487-493, 1991 and Houghton et al., Nature 354: 84-88, 1991). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90: 6909-6913, 1993), vinylogous polypeptides (Hagihara et al., J. Amer. Chem. Soc. 114: 6568, 1992), nonpeptidal peptidomimetics with glucose scaffolding (Hirschmann et al., J. Amer. Chem. Soc. 114: 9217-9218, 1992), analogous organic syntheses of small compound libraries (Chen et al., J. Amer. Chem. Soc. 116: 2661, 1994), oligocarbamates (Cho et al., Science 261: 1303, 1993), and/or peptidyl phosphonates (Campbell et al., J. Org. Chem. 59: 658, 1994), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14: 309-314, 1996 and PCT/US96/10287), carbohydrate libraries (see, e.g., Liang et al., Science 274: 1520-1522, 1996 and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

Candidate compounds are useful as part of a strategy to identify drugs for treating disorders involving TNF-α induction via pathways involving toll-like receptor 4/CD14 interaction or toll-like receptor 4/CD14/TRAM/Trif interaction. A test compound that binds to TLR4, CD14 or TRAM/Trif is considered a candidate compound.

Screening assays for identifying candidate or test compounds that bind to TLR4, CD14 or TRAM/Trif, or modulate the activity of TLR4, CD14 or TRAM/Trif proteins or polypeptides or biologically active portions thereof, are also included in the invention. The test compounds can be obtained using any of the numerous approaches in combinatorial library methods known in the art, including, but not limited to, biological libraries; spatially addressable parallel solid phase or solution phase libraries; synthetic library methods requiring deconvolution; the “one-bead one-compound” library method; and synthetic library methods using affinity chromatography selection. The biological library approach can be used for, e.g., peptide libraries, while the other four approaches are applicable to peptide, non-peptide oligomer or small molecule libraries of compounds (Lam, Anticancer Drug Des. 12: 145, 1997). Examples of methods for the synthesis of molecular libraries can be found in the art, for example in: DeWitt et al., Proc. Natl. Acad. Sci. U.S.A. 90: 6909, 1993; Erb et al., Proc. Natl. Acad. Sci. USA 91: 11422, 1994; Zuckermann et al., J. Med. Chem. 37: 2678, 1994; Cho et al., Science 261: 1303, 1993; Carrell et al., Angew. Chem. Int. Ed. Engl. 33: 2059, 1994; Carell et al., Angew. Chem. Int. Ed. Engl. 33: 2061, 1994; and Gallop et al., J. Med. Chem. 37: 1233, 1994. In some embodiments, the test compounds are dominant negative variants of TLR4, CD14 or TRAM/Trif.

Libraries of compounds can be presented in solution (e.g., Houghten, Bio/Techniques 13: 412-421, 1992), or on beads (Lam, Nature 354: 82-84, 1991), chips (Fodor, Nature 364: 555-556, 1993), bacteria (U.S. Pat. No. 5,223,409), spores (U.S. Pat. Nos. 5,571,698, 5,403,484, and 5,223,409), plasmids (Cull et al., Proc. Natl. Acad. Sci. USA 89: 1865-1869, 1992) or on phage (Scott et al., Science 249: 386-390, 1990; Devlin, Science 249: 404-406, 1990; Cwirla et al., Proc. Natl. Acad. Sci. USA 87: 6378-6382, 1990; and Felici, J. Mol. Biol. 222: 301-310, 1991).

The ability of a test compound to modulate the activity of TLR4, CD14 or TRAM/Trif or a biologically active portion thereof can be determined, e.g., by monitoring the ability to form toll-like receptor 4/CD14 complexes or toll-like receptor 4/CD14/TRAM/Trif complexes in the presence of the test compound. The ability of the test compound to modulate the activity of toll-like receptor 4 or a biologically active portion thereof can also be determined by monitoring the ability of the toll-like receptor 4 protein to bind to CD14. Such assays can be in the presence of TRAM/Trif. The binding assays can be cell-based or cell-free.

The ability of a toll-like receptor 4 protein to bind to or interact with CD14 and/or TRAM/Trif can be determined by one of the methods described herein or known in the art for determining direct binding. In one embodiment, the ability of the toll-like receptor 4 protein to bind to or interact with CD14 or TRAM/Trif can be determined by monitoring the induction of TNF-α. Detection of the TNF-α can include detection of the expression of a recombinant TNF-α that also encodes a detectable marker such as a FLAG sequence or a luciferase. This assay can be in addition to an assay of direct binding. In general, such assays are used to determine the ability of a test compound to affect the binding of toll-like receptor 4 protein to CD14 and/or TRAM/Trif.

In general, the ability of a test compound to bind to CD14; interfere with signaling through toll-like receptor 4, but not interfere with signaling through TRAM/Trif; or otherwise affect the induction of TNF-α expression is compared to a control in which the binding or induction of TNF-α expression is determined in the absence of the test compound. In some cases, a predetermined reference value is used. Such reference values can be determined relative to controls, in which case a test sample that is different from the reference would indicate that the compound binds to the molecule of interest (e.g., toll-like receptor 4) or modulates expression (e.g., activates or inhibits TNF-α activity in a cell that has been induced by lipopolysaccharide, or activates or inhibits macrophage response to vesicular stomatitis virus or rabies virus). A reference value can also reflect the amount of binding or induction of TNF-α expression observed with a standard (e.g., the affinity of antibody for toll-like receptor 4, or modulation of TNF-α expression by lipopolysaccharide). In this case, a test compound that is similar to (e.g., equal to or less than) the reference would indicate that compound is a candidate compound (e.g., binds to toll-like receptor 4 to a degree equal to or greater than a reference antibody).

This invention further pertains to novel agents identified by the above-described screening assays and uses thereof for treatments as described herein.

In one embodiment the invention provides soluble assays using CD14 or toll-like receptor 4 protein, or a cell or tissue expressing CD14 or toll-like receptor 4 protein, either naturally occurring or recombinant. In another embodiment, the invention provides solid phase based in vitro assays in a high throughput format, where CD14 or toll-like receptor 4 protein or its ligand is attached to a solid phase substrate via covalent or non-covalent interactions. Any one of the assays described herein can be adapted for high throughput screening.

In the high throughput assays of the invention, either soluble or solid state, it is possible to screen up to several thousand different modulators or ligands in a single day. This methodology can be used for CD14 or toll-like receptor 4 proteins in vitro, or for cell-based or membrane-based assays comprising CD14 or toll-like receptor 4 protein. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100- about 1500 different compounds. It is possible to assay many plates per day; assay screens for up to about 6,000, 20,000, 50,000, or more than 100,000 different compounds are possible using the integrated systems of the invention.

For a solid state reaction, the protein of interest or a fragment thereof, e.g., an extracellular domain, or a cell or membrane comprising the protein of interest or a fragment thereof as part of a fusion protein can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the F_{c} region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as toll-like receptors, transferrin, c-kit, viral receptor ligands, cytokine receptors, chemokine receptors, interleukin receptors, immunoglobulin receptors and antibodies, the cadherin family, the integrin family, the selectin family, and the like; see, e.g., Pigott & Power, The Adhesion Molecule Facts Book I, 1993. Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill upon review of this disclosure.

Common linkers such as peptides, polyethers, and the like can also serve as tags, and include polypeptide sequences, such as poly gly sequences of between about 5 and 200 amino acids. Such flexible linkers are known to persons of skill in the art. For example, polyethylene glycol linkers are available from Shearwater Polymers, Inc. Huntsville, Alabama. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85: 2149-2154, 1963 (describing solid phase synthesis of, e.g., peptides); Geysen et al., J. Immun. Meth. 102: 259-274, 1987 (describing synthesis of solid phase components on pins); Frank & Doring, Tetrahedron 44: 6031-6040, 1988 (describing synthesis of various peptide sequences on cellulose disks); Fodor et al., Science 251: 767-777, 1991; Sheldon et al., Clinical Chemistry 39: 718-719, 1993; and Kozal et al., Nature Medicine 2: 753-759, 1996 (all describing arrays of biopolymers fixed to solid substrates). Non-chemical approaches for fixing tag binders to substrates include other common methods, such as heat, cross-linking by UV radiation, and the like.

### Bispecific Compounds as Modulators of CD14 and Toll-Like Receptor 4

In one aspect, a method for identifying candidate or test bispecific compounds is provided which reduce the concentration of an agent in the serum and/or circulation of a non-human animal. Compounds selected or optimized using the instant methods can be used to treat subjects that would benefit from administration of such a compound, e.g., human subjects.

Candidate compounds that can be tested in an embodiment of the methods of the present invention are bispecific compounds. As used herein, the term “bispecific compound” includes compounds having two different binding specificities. Exemplary bispecific compounds include, e.g., bispecific antibodies, heteropolymers, and antigen-based heteropolymers.

Bispecific molecules that can be tested in an embodiment of the invention preferably include a binding moiety that is specific for CD14, preferably human CD14, crosslinked to a second binding moiety specific for a targeted agent (e.g. a distinct antibody or an antigen). Examples of binding moieties specific for toll-like receptor 4 include, but are not limited to, toll-like receptor 4 ligands, e.g. CD14 or, in preferred embodiments, antibodies to toll-like receptor 4.

In another embodiment, novel toll-like receptor 4 binding molecules can be identified based on their ability to bind to toll-like receptor 4. For example, libraries of compounds or small molecules can be tested cell-free binding assay. Any number of test compounds, e.g., peptidomimetics, small molecules or other drugs can be used for testing and can be obtained using any of the numerous approaches in combinatorial library methods known in the art, including: biological libraries; spatially addressable parallel solid phase or solution phase libraries; synthetic library methods requiring deconvolution; the ‘one-bead one-compound’ library method; and synthetic library methods using affinity chromatography selection. The biological library approach is limited to peptide libraries, while the other four approaches are applicable to peptide, non-peptide oligomer or small molecule libraries of compounds (Lam, Anticancer Drug Des. 12: 145, 1997).

In many drug screening programs which test libraries of modulating agents and natural extracts, high throughput assays are desirable in order to maximize the number of modulating agents surveyed in a given period of time. Assays which are performed in cell-free systems, such as can be derived with purified or semi-purified proteins, are often preferred as “primary” screens in that they can be generated to permit rapid development and relatively easy detection of an alteration in a molecular target which is mediated by a test modulating agent. Moreover, the effects of cellular toxicity and/or bioavailability of the test modulating agent can be generally ignored in the in vitro system, the assay instead being focused primarily on the effect of the drug on the molecular target as can be manifest in an alteration of binding affinity with upstream or downstream elements.

In another embodiment, phage display techniques known in the art can be used to identify novel TLR4, CD14 or TRAM/Trif binding molecules.

In one embodiment, the invention provides assays for screening candidate or test compounds which bind to TLR4, CD14 or TRAM/Trif or biologically active portion thereof.

Cell-based assays for identifying molecules that bind to TLR4, CD14 or TRAM/Trif can be used to identify additional agents for use in bispecific compounds of the invention. For example, cells expressing TLR4, CD14 or TRAM/Trif can be used in a screening assay. For example, compounds which produce a statistically significant change in binding to TLR4, CD14 or TRAM/Trif can be identified.

In one embodiment, the assay is a cell-free assay in which a toll-like receptor 4 binding molecule is identified based on its ability to bind to TLR4, CD14 or TRAM/Trif in vitro. The TLR4, CD14 or TRAM/Trif binding molecule can be provided and the ability of the protein to bind TLR4, CD14 or TRAM/Trif can be tested using art recognized methods for determining direct binding. Determining the ability of the protein to bind to a target molecule can be accomplished, e.g., using a technology such as real-time Biomolecular Interaction Analysis (BIA). Sjolander et al., Anal. Chem. 63: 2338-2345, 1991, and Szabo et al., Curr. Opin. Struct. Biol. 5: 699-705, 1995. As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

The cell-free assays of the present invention are amenable to use of both soluble and/or membrane-bound forms of proteins. In the case of cell-free assays in which a membrane-bound form a protein is used it can be desirable to utilize a solubilizing agent such that the membrane-bound form of the protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-14, Thesit®, Isotridecypoly(ethylene glycol ether)ₙ, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Suitable assays are known in the art that allow for the detection of protein-protein interactions (e.g., immunoprecipitations, two-hybrid assays and the like). By performing such assays in the presence and absence of test compounds, these assays can be used to identify compounds that modulate (e.g., inhibit or enhance) the interaction of a protein of the invention with a target molecule(s).

Determining the ability of the protein to bind to or interact with a target molecule can be accomplished, e.g., by direct binding. In a direct binding assay, the protein could be coupled with a radioisotope or enzymatic label such that binding of the protein to a target molecule can be determined by detecting the labeled protein in a complex. For example, proteins can be labeled with ¹²⁵I, ³⁵S, ¹⁴C, or ³H, either directly or indirectly, and the radioisotope detected by direct counting of radioemmission or by scintillation counting. Alternatively, molecules can be enzymatically labeled with, for example, horseradish peroxidase, alkaline phosphatase, or luciferase, and the enzymatic label detected by determination of conversion of an appropriate substrate to product.

Typically, it will be desirable to immobilize either a protein of the invention or its binding protein to facilitate separation of complexes from uncomplexed forms of one or both of the proteins, as well as to accommodate automation of the assay. Binding to an upstream or downstream binding element, in the presence and absence of a candidate agent, can be accomplished in any vessel suitable for containing the reactants. Examples include microtitre plates, test tubes, and micro-centrifuge tubes. In one embodiment, a fusion protein can be provided which adds a domain that allows the protein to be bound to a matrix. For example, glutathione-S-transferase/CD14 (GST/CD14) fusion proteins can be adsorbed onto glutathione sepharose beads (Sigma Chemical, St. Louis, Mo.) or glutathione derivatized microtitre plates, which are then combined with the cell lysates, e.g. ³⁵S-labeled, and the test modulating agent, and the mixture incubated under conditions conducive to complex formation, e.g., at physiological conditions for salt and pH, though slightly more stringent conditions can be used. Following incubation, the beads are washed to remove any unbound label, and the matrix immobilized and radiolabel determined directly (e.g. beads placed in scintilant), or in the supernatant after the complexes are subsequently dissociated. Alternatively, the complexes can be dissociated from the matrix, separated by SDS-PAGE, and the level of CD14 -binding protein found in the bead fraction quantitated from the gel using standard electrophoretic techniques.

Other techniques for immobilizing proteins on matrices are also available for use in the subject assay. For instance, biotinylated molecules can be prepared from biotin-NHS (N-hydroxy-succinimide) using techniques well known in the art (e.g., biotinylation kit, Pierce Chemicals, Rockford, Ill.), and immobilized in the wells of streptavidin-coated 96 well plates (Pierce Chemical).

It is also within the scope of this invention to determine the ability of a compound to modulate the interaction between TLR4, CD14 and TRAM/Trif, without the labeling of any of the interactants. For example, a microphysiometer can be used to detect the interaction of a protein of the invention with its target molecule without the labeling of either the protein or the target molecule. McConnell et al., Science 257: 1906-1912, 1992. As used herein, a “microphysiometer” (e.g., Cytosensor) is an analytical instrument that measures the rate at which a cell acidifies its environment using a light-addressable potentiometric sensor (LAPS). Changes in this acidification rate can be used as an indicator of the interaction between compound and receptor.

Antigen-based heteropolymers that can be tested in the present invention preferentially include a binding moiety that is specific for TLR4, CD14 or TRAM/Trif, preferably human TLR4, CD14 or TRAM/Trif, crosslinked to an antigen that is recognized by an autoantibody. Examples of antigens recognized by autoantibodies include, but are not limited to, any one of the following: factor VIII (antibodies associated with treatment of hemophilia by replacement recombinant factor VIII); the muscle acetylcholine receptor (the antibodies are associated with the disease myasthenia gravis); cardiolipin (associated with the disease lupus); platelet associated proteins (associated with the disease idiopathic thrombocytopenic purpura); the multiple antigens associated with Sjogren's Syndrome; the antigens implicated in the case of tissue transplantation autoimmune reactions; the antigens found on heart muscle (associated with the disease autoimmune myocarditis); the antigens associated with immune complex mediated kidney disease; the dsDNA and ssDNA antigens (associated with lupus nephritis); desmogleins and desmoplakins (associated with pemphigus and pemphigoid); or any other antigen which is well-characterized and is associated with disease pathogenesis.

Exemplary heteropolymers and antigen-based heteropolymers for testing in the instant invention and methods of making them are known in the art. For example, exemplary heteropolymers are taught in WO 03007971A1; U.S. 20020103343A1; U.S. Pat. No. 5,879,679; U.S. Pat. No. 5,487,890; U.S. Pat. No. 5,470,570; WO 9522977A1; WO/02075275A3, WO/0246208A2 or A3, W/0180883A1, WO/0145669A1, WO 9205801A1, Lindorfer et al., J. Immunol. Methods. 248: 125, 2001; Hahn et al., J. Immnol. 166: 1057, 2001; Nardin et al., J. Immunol. Methods. 211: 21, 1998; Kuhn et al., J. Immunol. 160: 5088, 1998; Taylor et al., Cancer Immunol. Immunother. 45: 152, 1997; Taylor et al., J. Immunol. 159: 4035, 1997; and Taylor et al., J. Immunol. 148: 2462, 1992. In addition, variant forms of these heteropolymers can be made. For example, in one embodiment, forms of bispecific molecules made using different linking chemistries can be used. Exemplary reagents that can be used to cross-link the components of a bispecific molecule include: polyethelyene glycol, SATA, SMCC, as well others known in the art, and available, e.g., from Pierce Biotechnology. Exemplary forms of bispecific molecules that can be tested are described in U.S. Ser. No. 60/411,731, filed on Sep. 16, 2002, the contents of which are incorporated herein by reference.

In another embodiment, different multimeric forms of bispecific molecules can be made (e.g., dimer, trimer, tetramer, pentamer, or higher multimer forms). In another embodiment, purified forms of bispecific molecules can be tested, e.g., as described in U.S. Ser. No. 60/380,211, filed on May 13, 2002, the contents of which are incorporated herein by reference.

In another embodiment, when one of the binding moieties of the heteropolymer is an antibody, antibodies of different isotypes (e.g., IgA, IgD, IgE, IgG1, IgG₂ (e.g., IgG₂a), IgG₃, IgG₄, or IgM) can be used. In another embodiment, portions of an antibody molecule (e.g., Fab fragments) can be used for one of the binding moieties. In a preferred embodiment at least one of the binding moieties is an antibody comprising an F_{c} domain. In one embodiment, the antibody is a mouse antibody.

In another embodiment, the effect of modifications to antibodies can be tested, e.g., the effect of deimmunization of the antibody, e.g., as described in U.S. Ser. No. 60/458,869, filed on Mar. 28, 2003 can be tested.

In methods provided in the present invention, the concentration of an agent, e.g. pathogenic agent, in the serum, circulation and/or tissue of the non-human animal can be reduced by at least e.g. about 20%, about 30%, about 40%, about 50%, about 60%, about 70%, about 80%, about 90% or about 100%.

In another embodiment, the concentration of an agent in the serum, circulation and/or tissue of a subject can be measured indirectly. For example, pathology resulting from the presence of the agent in the serum and/or circulation can be measured, e.g., by examining tissue samples from the animal. Another indirect measurement of the concentration of an agent in the serum, circulation and/or tissue of the non-human animal is measurement of the ability of the agent to cause infection in the non-human animal. For example, the effect of the bispecific compound on clinical signs and symptoms of infection can be measured. The ability of the bispecific compound to inhibit the spread of infection, e.g., from one organ system to another or from one individual to another can also be tested.

In another embodiment the ability of the bispecific compound to bind to cells bearing TLR4, CD14 or TRAM/Trif in the non-human animal is measured. For example, in one embodiment, determining the ability of the bispecific compound to bind to a TLR4, CD14 or TRAM/Trif target molecule can also be accomplished using a technology such as real-time Biomolecular Interaction Analysis (BIA) (Sjolander et al., Anal. Chem. 63: 2338-2345, 1991 and Szabo et al., Curr. Opin. Struct. Biol. 5: 699-705, 1995). As used herein, “BIA” is a technology for studying biospecific interactions in real time, without labeling any of the interactants (e.g., BIAcore). Changes in the optical phenomenon of surface plasmon resonance (SPR) can be used as an indication of real-time reactions between biological molecules.

In another embodiment, the destruction of the agent by cells in the non-human animal (e.g., killing by macrophage) is measured.

Compounds that reduce the concentration of the agent in the serum and/or circulation of the non-human animal (as compared with concentrations observed in non-human animals that do not receive the bispecific compound) can be selected.

Compounds for testing in the subject assays can be selected from among a plurality of compounds tested. In another embodiment, bispecific compounds for testing in the instant assays may have already been identified as being capable of binding TLR4, CD14 or TRAM/Trif, e.g., in an in vitro assay and can be further evaluated or optimized using the instant assays. In such cases, the ability of a bispecific compound to reduce the concentration of an agent in the serum and/or circulation can be compared to another bispecific compound or a non-optimized version of the same compound to determine its ability reduce the concentration of the agent in the serum and/or circulation.

In preferred embodiments, the bispecific compounds of the instant invention are administered at concentrations in the range of approximately 1 μg compound/kg of body weight to approximately 100 μg compound/kg of body weight. As defined herein, a therapeutically effective amount of a bispecific compound (i.e., an effective dosage) ranges from about 0.01 to 5000 μg/kg body weight, preferably about 0.1 to 500 μg/kg body weight, more preferably about 2 to 80 μg/kg body weight, and even more preferably about 5 to 70 μg/kg, 10 to 60 μg/kg, 20 to 50 μg/kg, 24 to 41 μg/kg, 25 to 40 μg/kg, 26 to 39 μg/kg, 27 to 38 μg/kg, 28 to 37 μg/kg, 29 to 36 μg/kg, 30 to 35 μg/kg, 31 to 34 μg/kg or 32 to 33 μg/kg body weight. The skilled artisan will appreciate that certain factors can influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments.

In a preferred example, the animal is treated with bispecific compound in the range of between about 1 to 500 μg/kg body weight following intravenous (iv) injection of an agent. It will also be appreciated that the effective dosage of a bispecific compound used for treatment can increase or decrease over the course of a particular treatment. Changes in dosage may result and become apparent from the results of diagnostic assays as described herein.

The route of administration of test compounds and/or agents can be intravenous (iv) injection into the circulation of the animal. Other administration routes include, but are not limited to, topical, parenteral, subcutaneous, or by inhalation. The term “parenteral” includes injection, e.g. by subcutaneous, intravenous, or intramuscular routes, also including localized administration, e.g., at a site of disease or injury. Sustained release of compounds from implants is also known in the art. One skilled in the pertinent art will recognize that suitable dosages will vary, depending upon such factors as the nature of the disorder to be treated, the patient's body weight, age, and general condition, and the route of administration. Preliminary doses can be determined according to animal tests, and the scaling of dosages for human administration are performed according to art-accepted practices.

The candidate compounds and agents can be administered over a range of doses to the animal. When the agent is also administered to the animal, the candidate compound can be administered either before, at the same time, or after, administration of the agent.

TLR4-, CD14-, or TRAM/Trif-expressing transgenic animals, e.g. mice, of the present invention can be used to screen or evaluate candidate compounds useful for treating disorders or diseases in humans that are associated with the presence of unwanted agents in the serum and/or circulation of a subject, such as autoantibodies, infectious agents, or toxins.

Exemplary targeted agents that can be bound by the bispecific compounds of the present invention include blood-borne agents, including, but not limited to, any of the following: viruses, viral particles, toxins, bacteria, polynucleotides, antibodies, e.g., autoantibodies associated with an autoimmune disorder. In one embodiment, exemplary targeted viral agents include, but are not limited to, any one of the following: cytomegalovirus, influenza, Newcastle disease virus, vesicular stomatitis virus, rabies virus, herpes simplex virus, hepatitis, adenovirus-2, bovine viral diarrhea virus, human immunodeficiency virus (HIV), dengue virus, Marburg virus, Epstein-Barr virus.

Exemplary bacterial agents include: Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacterjejuni, Campylobacter coli, Borrelia burgdorferi, Vibrio cholerae, Vibrio parahaemolyticus, Legionella pneumophila, Listeria monocytogenes, Neisseria gonorrhoeae, Neisseria meningitidis, Gardnerella vaginalis, Bacteroides fragilis, Bacteroides distasonis, Bacteroides 3452A homology group, Bacteroides vulgatus, Bacteroides ovalus, Bacteroides thetaiotaomnicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis, Staphylococcus saccharolyticus.

In one embodiment, the targeted agent is resistant to traditional therapies, e.g., is resistant to antibiotics.

In another embodiment, exemplary targeted agents that can be bound by the antigen-based heteropolymers of the present invention include, but are not limited to, any one of the following: autoantibodies associated with treatment of hemophilia by replacement recombinant factor VII; autoantibodies associated with the autoimmune diseases myasthenia gravis, lupus, lupus nephritis, idiopathic thrombocytopenic purpura, Sjogren's Syndrome, myocarditis, or pemphigus and pemphigoid; autoantibodies associated with tissue transplantation autoimmune reactions; autoantibodies associated with immune complex mediated kidney disease; or any other autoantibody which is well-characterized and is associated with disease pathogenesis.

In yet other embodiments, exemplary biologic agents that can be bound by the bispecific compounds of the present invention include infectious agents and toxins which can be associated with biowarfare, including, but not limited to, any one of the following: anthrax, smallpox, plague, Ebola, and Marburg virus.

In one embodiment, in performing an assay of the invention, the agent is administered to the transgenic animal, e.g., prior to, simultaneously with, or after administration of a bispecific compound.

The bispecific compounds of the present invention, or any portion thereof, can be modified to enhance their half life. Peptide analogs are commonly used in the pharmaceutical industry as non-peptide drugs with properties analogous to those of the template peptide. These types of non-peptide compounds are termed “peptide mimetics” or “peptidomimetics” (Fauchere, Adv. Drug Res. 15: 29, 1986; Veber et al., TINS p.392, 1985; and Evans et al., J. Med. Chem 30: 1229, 1987, which are incorporated herein by reference) and are usually developed with the aid of computerized molecular modeling. Peptide mimetics that are structurally similar to therapeutically useful peptides can be used to produce an equivalent therapeutic or prophylactic effect. Generally, peptidomimetics are structurally similar to a paradigm polypeptide (i.e., a polypeptide that has a biological or pharmacological activity), such as an antigen polypeptide, but have one or more peptide linkages optionally replaced by a linkage selected from the group consisting of: —CH₂NH—, —CH₂S—, —CH₂—CH₂—, —CH=CH— (cis and trans), —COCH₂—, —CH(OH)CH₂—, and —CH₂SO—, by methods known in the art and further described in the following references: Spatola, A. F. in Chemistry and Biochemistry of Amino Acids, Peptides, and Proteins Weinstein, B., ed., Marcel Dekker, New York, p. 267, 1983; Spatola, A. F., Vega Data, Vol. 1, Issue 3, “Peptide Backbone Modifications,” 1983; Morley, Trends. Pharm. Sci. pp.463-468, 1980; Hudson et al., Int. J. Pept. Prot. Res. 14: 177-185, 1979 (—CH₂NH—, CH₂CH₂—); Spatola et al., Life. Sci. 38: 1243-1249, 1986 (—CH₂—S); Hann, J. Chem. Soc. Perkin. Trans. 1: 307-314, 1982 (—CH—CH—, cis and trans); Almquist et al., J. Med. Chem. 23: 1392-1398, 1980 (—COCH₂—); Jennings-White et al., Tetrahedron Lett. 23: 2533, 1982 (—COCH₂—); Szelke et al., European Patent Application No. EP 45665 CA: 97: 39405, 1982 (—CH(OH)CH₂—); Holladay et al., Tetrahedron. Lett. 24: 4401-4404, 1983 (—C(OH)CH₂—); and Hruby, Life Sci. 31: 189-199, 1982 (—CH₂—S—); each of which is incorporated herein by reference. A particularly preferred non-peptide linkage is —CH₂NH—. Such peptide mimetics can have significant advantages over polypeptide embodiments, including, for example: more economical production, greater chemical stability, enhanced pharmacological properties (half-life, absorption, potency, efficacy, etc.), altered specificity (e.g., a broad-spectrum of biological activities), reduced antigenicity, and others. Labeling of peptidomimetics usually involves covalent attachment of one or more labels, directly or through a spacer (e.g., an amide group), to non-interfering position(s) on the peptidomimetic that are predicted by quantitative structure-activity data and/or molecular modeling. Such non-interfering positions generally are positions that do not form direct contacts with the macromolecules(s) to which the peptidomimetic binds to produce the therapeutic effect. Derivatization (e.g., labeling) of peptidomimetics should not substantially interfere with the desired biological or pharmacological activity of the peptidomimetic.

Systematic substitution of one or more amino acids of an amino acid sequence with a D-amino acid of the same type (e.g., D-lysine in place of L-lysine) can be used to generate more stable peptides. In addition, constrained peptides can be generated by methods known in the art (Rizo et al., Annu. Rev. Biochem. 61: 387, 1992, incorporated herein by reference); for example, by adding internal cysteine residues capable of forming intramolecular disulfide bridges which cyclize the peptide.

Such modified polypeptides can be produced in prokaryotic or eukaryotic host cells. Alternatively, such peptides can be synthesized by chemical methods. Methods for expression of heterologous polypeptides in recombinant hosts, chemical synthesis of polypeptides, and in vitro translation are well known in the art and are described further in Maniatis et al., Molecular Cloning: A Laboratory Manual, 2nd Ed., Cold Spring Harbor, N.Y., 1989; Berger et al., Methods in Enzymology, Volume 152, Guide to Molecular Cloning Techniques, 1987, Academic Press, Inc., San Diego, Calif.; Merrifield, J. Am. Chem. Soc. 91: 501, 1969; Chaiken, CRC Crit. Rev. Biochem. 11: 255, 1981; Kaiser et al., Science 243: 187, 1989; Merrifield, Science 232: 342, 1986; Kent, Annu. Rev. Biochem. 57: 957, 1988; and Offord, Semisynthetic Proteins, Wiley Publishing, 1980, which are incorporated herein by reference).

Polypeptides can be produced, typically by direct chemical synthesis, and used as a binding moiety of a heteropolymer. Peptides can be produced as modified peptides, with nonpeptide moieties attached by covalent linkage to the N-terminus and/or C-terminus. In certain preferred embodiments, either the carboxy-terminus or the amino-terminus, or both, are chemically modified. The most common modifications of the terminal amino and carboxyl groups are acetylation and amidation, respectively. Amino-terminal modifications such as acylation (e.g., acetylation) or alkylation (e.g., methylation) and carboxy-terminal modifications such as amidation, as well as other terminal modifications, including cyclization, can be incorporated into various embodiments of the test compounds. Certain amino-terminal and/or carboxy-terminal modifications and/or peptide extensions to the core sequence can provide advantageous physical, chemical, biochemical, and pharmacological properties, such as: enhanced stability, increased potency and/or efficacy, resistance to serum proteases, desirable pharmacokinetic properties, and others.

### Consturction of Transgenic Animals

In one aspect, the present invention provides a animal whose genome contains a polynucleotide encoding CD14 operably linked to a promoter such that the non-human or human TLR4, CD14 or TRAM/Trif gene is functionally expressed in the macrophages of the animal, or the non-human or human CD14 is a loss of function mutation in the macrophage of the animal. The present invention further provides methods for making a transgenic non-human animal expressing non-human or human CD14 in the macrophages of the animal.

The transgenic animal used in the methods of the invention can be, e.g., a mammal, a bird, a reptile or an amphibian. Suitable mammals for uses described herein include: rodents; ruminants; ungulates; domesticated mammals; and dairy animals. Preferred animals include: rodents, goats, sheep, camels, cows, pigs, horses, oxen, llamas, chickens, geese, and turkeys. In a preferred embodiment, the non-human animal is a mouse.

Various methods of making transgenic animals are known in the art (see, e.g., Watson, et al., “The Introduction of Foreign Genes Into Mice,” in Recombinant DNA, 2d Ed., W. H. Freeman & Co., New York, pp. 255-272, 1992; Gordon, Intl. Rev. Cytol. 115: 171-229, 1989; Jaenisch, Science 240: 1468-1474, 1989; Rossant, Neuron 2: 323-334, 1990). An exemplary protocol for the production of a transgenic pig can be found in White and Yannoutsos, Current Topics in Complement Research: 64th Forum in Immunology, pp. 88-94; U.S. Pat. No. 5,523,226; U.S. Pat. No. 5,573,933; PCT Application WO93/25071; and PCT Application WO95/04744. An exemplary protocol for the production of a transgenic rat can be found in Bader et al., Clinical and Experimental Pharmacology and Physiology, Supp. 3: S81-S87, 1996. An exemplary protocol for the production of a transgenic cow can be found in Transgenic Animal Technology, A Handbook, 1994, ed., Carl A. Pinkert, Academic Press, Inc. An exemplary protocol for the production of a transgenic sheep can be found in Transgenic Animal Technology, A Handbook, 1994, ed., Carl A. Pinkert, Academic Press, Inc. Several exemplary methods are set forth in more detail below.

A. Injection into the Pronucleus

Transgenic animals can be produced by introducing a nucleic acid construct according to the present invention into egg cells. The resulting egg cells are implanted into the uterus of a female for normal fetal development, and animals which develop and which carry the transgene are then backcrossed to create heterozygotes for the transgene. Embryonal target cells at various developmental stages are used to introduce the transgenes of the invention. Different methods are used depending on the stage of development of the embryonal target cell(s). Exemplary methods for introducing transgenes include, but are not limited to, microinjection of fertilized ovum or zygotes (Brinster et al., Proc. Natl. Acad. Sci. USA 82: 4438-4442, 1985), and viral integration (Jaenisch, Proc. Natl. Acad. Sci. USA 73: 1260-1264, 1976; Jahner et al., Proc. Natl. Acad. Sci. USA 82: 6927-6931, 1985; Van der Putten et al., Proc. Natl. Acad. Sci. USA 82: 6148-6152, 1985). Procedures for embryo manipulation and microinjection are described in, for example, Manipulating the Mouse Embryo (Cold Spring Harbor Laboratory Press, Cold Spring Harbor, NY., 1986, the contents of which are incorporated herein by reference). Similar methods are used for production of other transgenic animals.

In an exemplary embodiment, production of transgenic mice employs the following steps. Male and female mice, from a defined inbred genetic background, are mated. The mated female mice are previously treated with pregnant mare serum, PMS, to induce follicular growth and human chorionic gonadotropin, hCG, to induce ovulation. Following mating, the female is sacrificed and the fertilized eggs are removed from her uterine tubes. At this time, the pronuclei have not yet fused and it is possible to visualize them using light microscopy. In an alternative protocol, embryos can be harvested at varying developmental stages, e.g. blastocysts can be harvested. Embryos are recovered in a Dulbecco's modified phosphate buffered saline (DPBS) and maintained in Dulbecco's modified essential medium (DMEM) supplemented with 10% fetal bovine serum.

Foreign DNA or the recombinant construct (e.g. TLR4, CD14 or TRAM/Trif expression construct) is then microinjected (100-1000 molecules per egg) into a pronucleus. Microinjection of an expression construct can be performed using standard micro manipulators attached to a microscope. For instance, embryos are typically held in 100 microliter drops of DPBS under oil while being microinjected. DNA solution is microinjected into the male pronucleus. Successful injection is monitored by swelling of the pronucleus. Shortly thereafter, fusion of the pronuclei (a female pronucleus and a male pronucleus) occurs and, in some cases, foreign DNA inserts into (usually) one chromosome of the fertilized egg or zygote. Recombinant ES cells, which are prepared as set forth below, can be injected into blastocysts using similar techniques.

B. Embryonic Stem Cells

In another method of making transgenic mice, recombinant DNA molecules of the invention can be introduced into mouse embryonic stem (ES) cells. Resulting recombinant ES cells are then microinjected into mouse blastocysts using techniques similar to those set forth in the previous subsection.

ES cells are obtained from pre-implantation embryos and cultured in vitro (Evans et al., Nature 292: 154-156, 1981; Bradley et al., Nature 309: 255-258, 1984; Gossler et al., Proc. Natl. Acad. Sci. USA 83: 9065-9069, 1986; Robertson et al., Nature 322: 445-448, 1986). Any ES cell line that is capable of integrating into and becoming part of the germ line of a developing embryo, so as to create germ line transmission of the targeting construct, is suitable for use herein. For example, a mouse strain that can be used for production of ES cells is the 129J strain. A preferred ES cell line is murine cell line D3 (American Type Culture Collection catalog no. CRL 1934). The ES cells can be cultured and prepared for DNA insertion using methods known in the art and described in Robertson, Teratocarcinomas and Embryonic Stem Cells: A Practical Approach, E. J. Robertson, ed. IRL Press, Washington, D.C., 1987, in Bradley et al., Current Topics in Devel. Biol. 20: 357-371, 1986 and in Hogan et al., Manipulating the Mouse Embryo: A Laboratory Manual, Cold Spring Harbor Laboratory Press, Cold Spring Harbor, N.Y., 1986, the contents of which are incorporated herein by reference.

The expression construct can be introduced into the ES cells by methods known in the art, e.g., those described in Sambrook et al., Molecular Cloning: A Laboratory Manual, 2nd Ed., ed., Cold Spring Harbor laboratory Press: 1989, the contents of which are incorporated herein by reference. Suitable methods include, but are not limited to, electroporation, microinjection, and calcium phosphate treatment methods. The expression construct (e.g. TLR4, CD14 or TRAM/Trif) to be introduced into the ES cell is preferably linear. Linearization can be accomplished by digesting the DNA with a suitable restriction endonuclease selected to cut only within the vector sequence and not within the gene (e.g. TLR4, CD14 or TRAM/Trif gene).

After introduction of the expression construct, the ES cells are screened for the presence of the construct. The cells can be screened using a variety of methods. Where a marker gene is employed in the construct, the cells of the animal can be tested for the presence of the marker gene. For example, where the marker gene is an antibiotic resistance gene, the cells can be cultured in the presence of an otherwise lethal concentration of antibiotic (e.g. G418 to select for neo). Those cells that survive have presumably integrated the transgene construct. If the marker gene is a gene that encodes an enzyme whose activity can be detected (e.g., beta.-galactosidase), the enzyme substrate can be added to the cells under suitable conditions, and the enzymatic activity can be analyzed. Alternatively, or additionally, ES cell genomic DNA can be examined directly. For example, the DNA can be extracted from the ES cells using standard methods and the DNA can then be probed on a Southern blot with a probe or probes designed to hybridize specifically to the transgene. The genomic DNA can also be amplified by PCR with probes specifically designed to amplify DNA fragments of a particular size and sequence of the transgene such that, only those cells containing the targeting construct will generate DNA fragments of the proper size.

C. Implantation

The zygote harboring a recombinant nucleic acid molecule of the invention (e.g. TLR4, CD14 or TRAM/Trif) is implanted into a pseudo-pregnant female mouse that was obtained by previous mating with a vasectomized male. In a general protocol, recipient females are anesthetized, paralumbar incisions are made to expose the oviducts, and the embryos are transformed into the ampullary region of the oviducts. The body wall is sutured and the skin closed with wound clips. The embryo develops for the full gestation period, and the surrogate mother delivers the potentially transgenic mice. Finally, the newborn mice are tested for the presence of the foreign or recombinant DNA. Of the eggs injected, on average 10% develop properly and produce mice. Of the mice born, on average one in four (25%) are transgenic for an overall efficiency of 2.5%. Once these mice are bred they transmit the foreign gene in a normal (Mendelian) fashion linked to a mouse chromosome.

D. Screening for the Presence of the Transgenic Construct

Transgenic animals can be identified after birth by standard protocols. DNA from tail tissue can be screened for the presence of the transgene construct, e.g., using southern blots and/or PCR. Offspring that appear to be mosaics are then crossed to each other if they are believed to carry the transgene in order to generate homozygous animals. If it is unclear whether the offspring will have germ line transmission, they can be crossed with a parental or other strain and the offspring screened for heterozygosity. The heterozygotes are identified by southern blots and/or PCR amplification of the DNA. The heterozygotes can then be crossed with each other to generate homozygous transgenic offspring. Homozygotes can be identified by Southern blotting of equivalent amounts of genomic DNA from mice that are the product of this cross, as well as mice that are known heterozygotes and wild type mice. Probes to screen the southern blots can be designed based on the sequence of the human or non-human TLR4, CD14 or TRAM/Trif gene, or the marker gene, or both.

Other means of identifying and characterizing the transgenic offspring are known in the art. For example, western blots can be used to assess the level of expression of the gene introduced in various tissues of these offspring by probing the western blot with an antibody against the protein encoded by the gene introduced (e.g., the human or non-human TLR4, CD14 or TRAM/Trif protein), or an antibody against the marker gene product, where this gene is expressed.

In situ analysis, such as fixing the cells and labeling with an antibody, and/or FACS (fluorescence activated cell sorting) analysis of various cells, e.g. erythrocytes, from the offspring can be performed using suitable antibodies to look for the presence or absence of the transgene product. For example, to verify expression of TLR4, CD14 or TRAM/Trif in macrophages, flow cytometry can be performed using antibodies specific for human C TLR4, CD14 or TRAM/Trif R1, that are directly conjugated or used in conjunction with a secondary antibody that is fluorophore-conjugated and recognizes the antibody for TLR4, CD14 or TRAM/Trif. In this analysis, human erythrocytes can be used as a positive control and normal mouse erythrocytes can be used as a negative control for the presence of TLR4, CD14 or TRAM/Trif.

E. Mice Containing Multiple Transgenes or an Additional Mutation

Transgenic mice expressing TLR4, CD14 or TRAM/Trif on their circulating erythrocytes as described herein can be crossed with mice that a) harbor additional transgene(s), or b) contain mutations in other genes. Mice that are heterozygous or homozygous for each of the mutations can be generated and maintained using standard crossbreeding procedures. Examples of mice that can be bred with mice containing a CD14 transgene include, but are not limited to, mouse strains which are more prone to an auto-immune disease, such as mouse strains which are models for Lupus, e.g. mouse strains NZB/W, MRL+ or SJL.

The invention further pertains to cells derived from transgenic animals. Because certain modifications can occur in succeeding generations due to either mutation or environmental influences, such progeny may not, in fact, be identical to the parent cell, but are still included within the scope of the term as used herein.

### Recombinant Nucleic Acid Techniques

The nucleic acids used to practice this invention, whether RNA, iRNA, antisense nucleic acid, cDNA, genomic DNA, vectors, viruses or hybrids thereof, can be isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems.

Alternatively, these nucleic acids can be synthesized in vitro by well-known chemical synthesis techniques, as described in, e.g., Adams, J. Am. Chem. Soc. 105: 661, 1983; Belousov, Nucleic Acids Res. 25: 3440-3444, 1997; Frenkel, Free Radic. Biol. Med. 19: 373-380, 1995; Blommers, Biochemistry 33: 7886-7896, 1994; Narang, Meth. Enzymol. 68: 90, 1979; Brown Meth. Enzymol. 68: 109, 1979; Beaucage, Tetra. Lett. 22: 1859, 1981; U.S. Pat. No. 4,458,066.

The invention provides oligonucleotides comprising sequences of the invention, e.g., subsequences of the exemplary sequences of the invention. Oligonucleotides can include, e.g., single stranded poly-deoxynucleotides or two complementary polydeoxynucleotide strands which can be chemically synthesized.

Techniques for the manipulation of nucleic acids, such as, e.g., subcloning, labeling probes (e.g., random-primer labeling using Klenow polymerase, nick translation, amplification), sequencing, hybridization and the like are well described in the scientific and patent literature, see, e.g., Sambrook, ed., MOLECULAR CLONING: A LABORATORY MANUAL (2ND ED.), Vols. 1-3, Cold Spring Harbor Laboratory, 1989; CURRENT PROTOCOLS IN MOLECULAR BIOLOGY, Ausubel, ed. John Wiley & Sons, Inc., New York, 1997; LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y., 1993.

Nucleic acids, vectors, capsids, polypeptides, and the like can be analyzed and quantified by any of a number of general means well known to those of skill in the art. These include, e.g., analytical biochemical methods such as NMR, spectrophotometry, radiography, electrophoresis, capillary electrophoresis, high performance liquid chromatography (HPLC), thin layer chromatography (TLC), and hyperdiffusion chromatography, various immunological methods, e.g. fluid or gel precipitin reactions, immunodiffusion, immuno-electrophoresis, adioimmunoassay (RIAs), enzyme-linked immunosorbent assays (ELISAs), immuno-fluorescent assays, Southern analysis, Northern analysis, dot-blot analysis, gel electrophoresis (e.g., SDS-PAGE), nucleic acid or target or signal amplification methods, radiolabeling, scintillation counting, and affinity chromatography.

Obtaining and manipulating nucleic acids used to practice the methods of the invention can be done by cloning from genomic samples, and, if desired, screening and re-cloning inserts isolated or amplified from, e.g., genomic clones or cDNA clones. Sources of nucleic acid used in the methods of the invention include genomic or cDNA libraries contained in, e.g., mammalian artificial chromosomes (MACs), see, e.g., U.S. Pat. Nos. 5,721,118; 6,025,155; human artificial chromosomes, see, e.g., Rosenfeld, Nat. Genet. 15: 333-335, 1997; yeast artificial chromosomes (YAC); bacterial artificial chromosomes (BAC); P1 artificial chromosomes, see, e.g., Woon, Genomics 50: 306-316, 1998; P1-derived vectors (PACs), see, e.g., Kern, Biotechniques 23:120-124, 1997; cosmids, recombinant viruses, phages or plasmids.

The invention provides fusion proteins and nucleic acids encoding them. A CD14 or toll-like receptor 4 polypeptide can be fused to a heterologous peptide or polypeptide, such as N-terminal identification peptides which impart desired characteristics, such as increased stability or simplified purification. Peptides and polypeptides of the invention can also be synthesized and expressed as fusion proteins with one or more additional domains linked thereto for, e.g., producing a more immunogenic peptide, to more readily isolate a recombinantly synthesized peptide, to identify and isolate antibodies and antibody-expressing B cells, and the like. Detection and purification facilitating domains include, e.g., metal chelating peptides such as polyhistidine tracts and histidine-tryptophan modules that allow purification on immobilized metals, protein A domains that allow purification on immobilized immunoglobulin, and the domain utilized in the FLAGS extension/affinity purification system (Immunex Corp, Seattle Wash.). The inclusion of a cleavable linker sequences such as Factor Xa or enterokinase (Invitrogen, San Diego Calif.) between a purification domain and the motif-comprising peptide or polypeptide to facilitate purification. For example, an expression vector can include an epitope-encoding nucleic acid sequence linked to six histidine residues followed by a thioredoxin and an enterokinase cleavage site (see e.g., Williams, Biochemistry 34: 1787-1797, 1995; Dobeli, Protein Expr. Purif 12: 404-414, 1998). The histidine residues facilitate detection and purification while the enterokinase cleavage site provides a means for purifying the epitope from the remainder of the fusion protein. In one aspect, a nucleic acid encoding a polypeptide of the invention is assembled in appropriate phase with a leader sequence capable of directing secretion of the translated polypeptide or fragment thereof. Technology pertaining to vectors encoding fusion proteins and application of fusion proteins are well described in the scientific and patent literature, see e.g., Kroll, DNA Cell. Biol. 12: 441-53, 1993.

A. Transcriptional Control Elements

The nucleic acids of the invention can be operatively linked to a promoter. A promoter can be one motif or an array of nucleic acid control sequences which direct transcription of a nucleic acid. A promoter can include necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription. A “constitutive” promoter is a promoter which is active under most environmental and developmental conditions. An “inducible” promoter is a promoter which is under environmental or developmental regulation. A “tissue specific” promoter is active in certain tissue types of an organism, but not in other tissue types from the same organism. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

B. Expression Vectors And Cloning Vehicles

The invention provides expression vectors and cloning vehicles comprising nucleic acids of the invention, e.g., sequences encoding the proteins of the invention. Expression vectors and cloning vehicles of the invention can comprise viral particles, baculovirus, phage, plasmids, phagemids, cosmids, fosmids, bacterial artificial chromosomes, viral DNA (e.g., vaccinia, adenovirus, foul pox virus, pseudorabies and derivatives of SV40), P1-based artificial chromosomes, yeast plasmids, yeast artificial chromosomes, and any other vectors specific for specific hosts of interest (such as bacillus, Aspergillus and yeast). Vectors of the invention can include chromosomal, non-chromosomal and synthetic DNA sequences. Large numbers of suitable vectors are known to those of skill in the art, and are commercially available.

The nucleic acids of the invention can be cloned, if desired, into any of a variety of vectors using routine molecular biological methods; methods for cloning in vitro amplified nucleic acids are described, e.g., U.S. Pat. No. 5,426,039. To facilitate cloning of amplified sequences, restriction enzyme sites can be “built into” a PCR primer pair.

The invention provides libraries of expression vectors encoding polypeptides and peptides of the invention. These nucleic acids can be introduced into a genome or into the cytoplasm or a nucleus of a cell and expressed by a variety of conventional techniques, well described in the scientific and patent literature. See, e.g., Roberts, Nature 328: 731, 1987; Schneider, Protein Expr. Purif. 6435: 10, 1995; Sambrook, Tijssen or Ausubel. The vectors can be isolated from natural sources, obtained from such sources as ATCC or GenBank libraries, or prepared by synthetic or recombinant methods. For example, the nucleic acids of the invention can be expressed in expression cassettes, vectors or viruses which are stably or transiently expressed in cells (e.g., episomal expression systems). Selection markers can be incorporated into expression cassettes and vectors to confer a selectable phenotype on transformed cells and sequences. For example, selection markers can code for episomal maintenance and replication such that integration into the host genome is not required.

In one aspect, the nucleic acids of the invention are administered in vivo for in situ expression of the peptides or polypeptides of the invention. The nucleic acids can be administered as “naked DNA” (see, e.g., U.S. Pat. No. 5,580,859) or in the form of an expression vector, e.g., a recombinant virus. The nucleic acids can be administered by any route, including peri- or intra-tumorally, as described below. Vectors administered in vivo can be derived from viral genomes, including recombinantly modified enveloped or non-enveloped DNA and RNA viruses, preferably selected from baculoviridiae, parvoviridiae, picornoviridiae, herpesveridiae, poxyiridae, adenoviridiae, or picornnaviridiae. Chimeric vectors can also be employed which exploit advantageous merits of each of the parent vector properties (See e.g., Feng, Nature Biotechnology 15: 866-870, 1997). Such viral genomes can be modified by recombinant DNA techniques to include the nucleic acids of the invention; and can be further engineered to be replication deficient, conditionally replicating or replication competent. In alternative aspects, vectors are derived from the adenoviral (e.g., replication incompetent vectors derived from the human adenovirus genome, see, e.g., U.S. Pat. Nos. 6,096,718; 6,110,458; 6,113,913; 5,631,236); adeno-associated viral and retroviral genomes. Retroviral vectors can include those based upon murine leukemia virus (MuLV), gibbon ape leukemia virus (GaLV), Simian Immuno deficiency virus (SIV), human immuno deficiency virus (HIV), and combinations thereof; see, e.g., U.S. Pat. Nos. 6,117,681; 6,107,478; 5,658,775; 5,449,614; Buchscher, J. Virol. 66: 2731-2739, 1992; Johann, J. Virol. 66: 1635-1640, 1992). Adeno-associated virus (AAV)-based vectors can be used to adioimmun cells with target nucleic acids, e.g., in the in vitro production of nucleic acids and peptides, and in in vivo and ex vivo gene therapy procedures; see, e.g., U.S. Pat. Nos. 6,110,456; 5,474,935; Okada, Gene Ther. 3: 957-964, 1996.

“Expression cassette” as used herein refers to a nucleotide sequence which is capable of affecting expression of a structural gene (i.e., a protein coding sequence, such as a polypeptide of the invention) in a host compatible with such sequences. Expression cassettes include at least a promoter operably linked with the polypeptide coding sequence; and, optionally, with other sequences, e.g., transcription termination signals. Additional factors necessary or helpful in effecting expression can also be used, e.g., enhancers.

A nucleic acid is “operably linked” when it is placed into a functional relationship with another nucleic acid sequence. For instance, a promoter or enhancer is operably linked to a coding sequence if it affects the transcription of the sequence. With respect to transcription regulatory sequences, operably linked means that the DNA sequences being linked are contiguous and, where necessary to join two protein coding regions, contiguous and in reading frame. For switch sequences, operably linked indicates that the sequences are capable of effecting switch recombination. Thus, expression cassettes also include plasmids, expression vectors, recombinant viruses, any form of recombinant “naked DNA” vector, and the like.

“Vector” is intended to refer to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

C. Host Cells and Transformed Cells

The invention also provides a transformed cell comprising a nucleic acid sequence of the invention, e.g., a sequence encoding a polypeptide of the invention, or a vector of the invention. The host cell can be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Streptomyces, Bacillus subtilis, Salmonella typhimurium and various species within the genera Pseudomonas, Streptomyces, and Staphylococcus. Exemplary insect cells include Drosophila S2 and Spodoptera Sf9. Exemplary animal cells include CHO, COS or Bowes melanoma or any mouse or human cell line. The selection of an appropriate host is within the abilities of those skilled in the art.

The vector can be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation.

Engineered host cells can be cultured in conventional nutrient media modified as appropriate for activating promoters, selecting transformants or amplifying the genes of the invention. Following transformation of a suitable host strain and growth of the host strain to an appropriate cell density, the selected promoter can be induced by appropriate means (e.g., temperature shift or chemical induction) and the cells can be cultured for an additional period to allow them to produce the desired polypeptide or fragment thereof.

Cells can be harvested by centrifugation, disrupted by physical or chemical means, and the resulting crude extract is retained for further purification. Microbial cells employed for expression of proteins can be disrupted by any convenient method, including freeze-thaw cycling, sonication, mechanical disruption, or use of cell lysing agents. Such methods are well known to those skilled in the art. The expressed polypeptide or fragment can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the polypeptide. If desired, high performance liquid chromatography (HPLC) can be employed for final purification steps.

Various mammalian cell culture systems can also be employed to express recombinant protein. Examples of mammalian expression systems include the COS-7 lines of monkey kidney fibroblasts and other cell lines capable of expressing proteins from a compatible vector, such as the C127, 3T3, CHO, HeLa and BHK cell lines.

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue.

Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof. In some aspects, the DNA construct can be linearized prior to conducting an in vitro transcription reaction. The transcribed mRNA is then incubated with an appropriate cell-free translation extract, such as a rabbit reticulocyte extract, to produce the desired polypeptide or fragment thereof.

The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli.

D. Amplification of Nucleic Acids

In practicing the invention, nucleic acids encoding the polypeptides of the invention, or modified nucleic acids, can be reproduced by, e.g., amplification. The invention provides amplification primer sequence pairs for amplifying nucleic acids encoding polypeptides of the invention, e.g., primer pairs capable of amplifying nucleic acid sequences comprising the CD14 protein or toll-like receptor 4 sequences, or subsequences thereof.

Amplification methods include, e.g., polymerase chain reaction, PCR (PCR PROTOCOLS, A GUIDE TO METHODS AND APPLICATIONS, ed. Innis, Academic Press, N.Y., 1990 and PCR STRATEGIES, 1995, ed. Innis, Academic Press, Inc., N.Y., ligase chain reaction (LCR) (see, e.g., Wu, Genomics 4: 560, 1989; Landegren, Science 241: 1077, 1988; Barringer, Gene 89: 117, 1990); transcription amplification (see, e.g., Kwoh, Proc. Natl. Acad. Sci. USA 86: 1173, 1989); and, self-sustained sequence replication (see, e.g., Guatelli, Proc. Natl. Acad. Sci. USA 87: 1874, 1990); Q Beta replicase amplification (see, e.g., Smith, J. Clin. Microbiol. 35: 1477-1491, 1997), automated Q-beta replicase amplification assay (see, e.g., Burg, Mol. Cell. Probes 10: 257-271, 1996) and other RNA polymerase mediated techniques (e.g., NASBA, Cangene, Mississauga, Ontario); see also Berger, Methods Enzymol. 152: 307-316, 1987; Sambrook; Ausubel; U.S. Pat. Nos. 4,683,195 and 4,683,202; Sooknanan, Biotechnology 13: 563-564, 1995.

E. Hybridization of Nucleic Acids

The invention provides isolated or recombinant nucleic acids that hybridize under stringent conditions to an exemplary sequence of the invention, e.g., a CD14 sequence or toll-like receptor 4 sequence, or the complement of any thereof, or a nucleic acid that encodes a polypeptide of the invention. In alternative aspects, the stringent conditions are highly stringent conditions, medium stringent conditions or low stringent conditions, as known in the art and as described herein. These methods can be used to isolate nucleic acids of the invention.

In alternative aspects, nucleic acids of the invention as defined by their ability to hybridize under stringent conditions can be between about five residues and the full length of nucleic acid of the invention; e.g., they can be at least 5, 10, 15, 20, 25, 30, 35, 40, 50, 55, 60, 65, 70, 75, 80, 90, 100, 150, 200, 250, 300, 350, 400, 450, 500, 550, 600, 650, 700, 750, 800 or more residues in length, or, the full length of a gene or coding sequence, e.g., cDNA. Nucleic acids shorter than full length are also included. These nucleic acids can be useful as, e.g., hybridization probes, labeling probes, PCR oligonucleotide probes, iRNA, antisense or sequences encoding antibody binding peptides (epitopes), motifs, active sites and the like.

“Selectively (or specifically) hybridizes to” refers to the binding, duplexing, or hybridizing of a molecule to a particular nucleotide sequence under stringent hybridization conditions when that sequence is present in a complex mixture (e.g., total cellular or library DNA or RNA), wherein the particular nucleotide sequence is detected at least at about 10 times background. In one embodiment, a nucleic acid can be determined to be within the scope of the invention by its ability to hybridize under stringent conditions to a nucleic acid otherwise determined to be within the scope of the invention (such as the exemplary sequences described herein).

“Stringent hybridization conditions” refers to conditions under which a probe will hybridize to its target subsequence, typically in a complex mixture of nucleic acid, but not to other sequences in significant amounts (a positive signal (e.g., identification of a nucleic acid of the invention) is about 10 times background hybridization). Stringent conditions are sequence-dependent and will be different in different circumstances. Longer sequences hybridize specifically at higher temperatures. An extensive guide to the hybridization of nucleic acids is found in e.g., Sambrook, ed., MOLECULAR CLONING: A LABORATORY MANUAL (2ND ED.), Vols. 1-3, Cold Spring Harbor Laboratory, 1989; CURRENT PROTOCOLS IN MOLECULAR BIOLOGY, Ausubel, ed. John Wiley & Sons, Inc., New York, 1997; LABORATORY TECHNIQUES IN BIOCHEMISTRY AND MOLECULAR BIOLOGY: HYBRIDIZATION WITH NUCLEIC ACID PROBES, PART I. Theory and Nucleic Acid Preparation, Tijssen, ed. Elsevier, N.Y., 1993.

Generally, stringent conditions are selected to be about 5-10C lower than the thermal melting point I for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions can also be achieved with the addition of destabilizing agents such as formamide as described in Sambrook (cited below). For high stringency hybridization, a positive signal is at least two times background, preferably 10 times background hybridization. Exemplary high stringency or stringent hybridization conditions include: 50% formamide, 5×SSC and 1% SDS incubated at 42° C. or 5×SSC and 1% SDS incubated at 65° C., with a wash in 0.2×SSC and 0.1% SDS at 65° C. For selective or specific hybridization, a positive signal (e.g., identification of a nucleic acid of the invention) is about 10 times background hybridization. Stringent hybridization conditions that are used to identify nucleic acids within the scope of the invention include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. In the present invention, genomic DNA or cDNA comprising nucleic acids of the invention can be identified in standard Southern blots under stringent conditions using the nucleic acid sequences disclosed here. Additional stringent conditions for such hybridizations (to identify nucleic acids within the scope of the invention) are those which include a hybridization in a buffer of 40% formamide, 1 M NaCl, 1% SDS at 37° C.

However, the selection of a hybridization format is not critical—it is the stringency of the wash conditions that set forth the conditions which determine whether a nucleic acid is within the scope of the invention. Wash conditions used to identify nucleic acids within the scope of the invention include, e.g., a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. See Sambrook, Tijssen and Ausubel for a description of SSC buffer and equivalent conditions.

F. Oligonucleotides Probes and Methods for Using Them

The invention also provides nucleic acid probes for identifying nucleic acids encoding a polypeptide which is a modulator of a TLR4-signaling activity. In one aspect, the probe comprises at least 10 consecutive bases of a nucleic acid of the invention. Alternatively, a probe of the invention can be at least about 5, 6, 7, 8, 9, 10, 15, 20, 25, 30, 35, 40, 45, 50, 60, 70, 80, 90, 100, 110, 120, 130, 150 or about 10 to 50, about 20 to 60 about 30 to 70, consecutive bases of a sequence as set forth in a nucleic acid of the invention. The probes identify a nucleic acid by binding and/or hybridization. The probes can be used in arrays of the invention, see discussion below. The probes of the invention can also be used to isolate other nucleic acids or polypeptides.

G. Determining the Degree of Sequence Identity

The invention provides nucleic acids having at least 90%, 91%, 92%, 93%, 94%, 95%, 96%, 97%, 98%, 99% or more sequence identity to CD14 polynucleotide or toll-like receptor 4 polynucleotide. The invention provides polypeptides having at least 90%, 91%, 92%, 93%, 94%, 95%, 96%, 97%, 98%, 99% or more sequence identity to CD14 protein or toll-like receptor 4 protein. The sequence identities can be determined by analysis with a sequence comparison algorithm or by a visual inspection. Protein and/or nucleic acid sequence identities (homologies) can be evaluated using any of the variety of sequence comparison algorithms and programs known in the art.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins, the BLAST and BLAST 2.2.2. or FASTA version 3.0t78 algorithms and the default parameters discussed below can be used.

A “comparison window”, as used herein, includes reference to a segment of any one of the number of contiguous positions selected from the group consisting of from 20 to 600, usually about 50 to about 200, more usually about 100 to about 150 in which a sequence can be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2: 482, 1981, by the homology alignment algorithm of Needleman & Wunsch, J. Mol. Biol. 48: 443, 1970, by the search for similarity method of Pearson & Lipman, Proc. Natl. Acad. Sci. U.S.A. 85: 2444, 1988, by computerized implementations of these algorithms (FASTDB (Intelligenetics), BLAST (National Center for Biomedical Information), GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., (1999 Suppl.), Current Protocols in Molecular Biology, Greene Publishing Associates and Wiley Interscience, N.Y., 1987)

A preferred example of an algorithm that is suitable for determining percent sequence identity and sequence similarity is the FASTA algorithm, which is described in Pearson & Lipman, Proc. Natl. Acad. Sci. U.S.A. 85: 2444, 1988. See also Pearson, Methods Enzymol. 266: 227-258, 1996. Preferred parameters used in a FASTA alignment of DNA sequences to calculate percent identity are optimized, BL50 Matrix 15: -5, k-tuple=2; joining penalty=40, optimization=28; gap penalty=−12, gap length penalty =-2; and width=16.

Another preferred example of algorithm that is suitable for determining percent sequence identity and sequence similarity are the BLAST and BLAST 2.0 algorithms, which are described in Altschul et al., Nuc. Acids Res. 25: 3389-3402, 1977; and Altschul et al., J. Mol. Biol. 215: 403-410, 1990, respectively. BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. U.S.A. 89:10915, 1989) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Natl. Acad. Sci. U.S.A. 90: 5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Another example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35: 351-360, 1987. The method used is similar to the method described by Higgins & Sharp, CABIOS 5:151-153, 1989. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. Using PILEUP, a reference sequence is compared to other test sequences to determine the percent sequence identity relationship using the following parameters: default gap weight (3.00), default gap length weight (0.10), and weighted end gaps. PILEUP can be obtained from the GCG sequence analysis software package, e.g., version 7.0 (Devereaux et al., Nuc. Acids Res. 12: 387-395, 1984.

Another preferred example of an algorithm that is suitable for multiple DNA and amino acid sequence alignments is the CLUSTALW program (Thompson et al., Nucl. Acids. Res. 22: 4673-4680, 1994). ClustaLW performs multiple pairwise comparisons between groups of sequences and assembles them into a multiple alignment based on homology. Gap open and Gap extension penalties were 10 and 0.05 respectively. For amino acid alignments, the BLOSUM algorithm can be used as a protein weight matrix (Henikoff and Henikoff, Proc. Natl. Acad. Sci. U.S.A. 89: 10915-10919, 1992).

“Sequence identity” refers to a measure of similarity between amino acid or nucleotide sequences, and can be measured using methods known in the art, such as those described below:

“Identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 80%, 85%, 90%, 91%, 92%, 93%, 94%, 95%, 96%, 97%, 98%, or 99% or more identity over a specified region, when compared and aligned for maximum correspondence over a comparison window, or designated region as measured using one of the following sequence comparison algorithms or by manual alignment and visual inspection.

“Substantially identical,” in the context of two nucleic acids or polypeptides, refers to two or more sequences or subsequences that have at least of at least 60%, often at least 70%, preferably at least 80%, most preferably at least 90% or at least 95% nucleotide or amino acid residue identity, when compared and aligned for maximum correspondence, as measured using one of the following sequence comparison algorithms or by visual inspection. Preferably, the substantial identity exists over a region of the sequences that is at least about 50 bases or residues in length, more preferably over a region of at least about 100 bases or residues, and most preferably the sequences are substantially identical over at least about 150 bases or residues. In a most preferred embodiment, the sequences are substantially identical over the entire length of the coding regions.

“Homology” and “identity” in the context of two or more nucleic acids or polypeptide sequences, refer to two or more sequences or subsequences that are the same or have a specified percentage of amino acid residues or nucleotides that are the same when compared and aligned for maximum correspondence over a comparison window or designated region as measured using any number of sequence comparison algorithms or by manual alignment and visual inspection. For sequence comparison, one sequence can act as a reference sequence (an exemplary sequence of CD14 or toll-like receptor 4 polynucleotide or polypeptide) to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the numbers of contiguous residues. For example, in alternative aspects of the invention, continugous residues ranging anywhere from 20 to the full length of an exemplary polypeptide or nucleic acid sequence of the invention, e.g., CD14 or toll-like receptor 4 polynucleotide or polypeptide, are compared to a reference sequence of the same number of contiguous positions after the two sequences are optimally aligned. If the reference sequence has the requisite sequence identity to an exemplary polypeptide or nucleic acid sequence of the invention, e.g., at least 90%, 91%, 92%, 93%, 94%, 95%, 96%, 97%, 98%, 99% or more sequence identity to CD14 or toll-like receptor 4 polynucleotide or polypeptide, that sequence is within the scope of the invention.

Motifs which can be detected using the above programs include sequences encoding leucine zippers, helix-turn-helix motifs, glycosylation sites, ubiquitination sites, alpha helices, and beta sheets, signal sequences encoding signal peptides which direct the secretion of the encoded proteins, sequences implicated in transcription regulation such as homeoboxes, acidic stretches, enzymatic active sites, substrate binding sites, and enzymatic cleavage sites.

H. Computer Systems and Computer Program Products

To determine and identify sequence identities, structural homologies, motifs and the like in silico, the sequence of the invention can be stored, recorded, and manipulated on any medium which can be read and accessed by a computer. Accordingly, the invention provides computers, computer systems, computer readable mediums, computer programs products and the like recorded or stored thereon the nucleic acid and polypeptide sequences of the invention. As used herein, the words “recorded” and “stored” refer to a process for storing information on a computer medium. A skilled artisan can readily adopt any known methods for recording information on a computer readable medium to generate manufactures comprising one or more of the nucleic acid and/or polypeptide sequences of the invention.

Another aspect of the invention is a computer readable medium having recorded thereon at least one nucleic acid and/or polypeptide sequence of the invention. Computer readable media include magnetically readable media, optically readable media, electronically readable media and magnetic/optical media. For example, the computer readable media can be a hard disk, a floppy disk, a magnetic tape, CD-ROM, Digital Versatile Disk (DVD), Random Access Memory (RAM), or Read Only Memory (ROM) as well as other types of other media known to those skilled in the art.

As used herein, the terms “computer,” “computer program” and “processor” are used in their broadest general contexts and incorporate all such devices.

### Inhibiting Expression of Polypoptides and Transcripts

The invention further provides for nucleic acids complementary to (e.g., antisense sequences to) the nucleic acid sequences of the invention. Antisense sequences are capable of inhibiting the transport, splicing or transcription of protein-encoding genes, e.g., CD14-encoding nucleic acids. The inhibition can be effected through the targeting of genomic DNA or messenger RNA. The transcription or function of targeted nucleic acid can be inhibited, for example, by hybridization and/or cleavage. One particularly useful set of inhibitors provided by the present invention includes oligonucleotides which are able to either bind gene or message, in either case preventing or inhibiting the production or function of the protein. The association can be through sequence specific hybridization. Another useful class of inhibitors includes oligonucleotides which cause inactivation or cleavage of protein message. The oligonucleotide can have enzyme activity which causes such cleavage, such as ribozymes. The oligonucleotide can be chemically modified or conjugated to an enzyme or composition capable of cleaving the complementary nucleic acid. One can screen a pool of many different such oligonucleotides for those with the desired activity.

General methods of using antisense, ribozyme technology and RNAI technology, to control gene expression, or of gene therapy methods for expression of an exogenous gene in this manner are well known in the art. Each of these methods utilizes a system, such as a vector, encoding either an antisense or ribozyme transcript of a phosphatase polypeptide of the invention. The term “RNAi” stands for RNA interference. This term is understood in the art to encompass technology using RNA molecules that can silence genes. See, for example, McManus, et al. Nature Reviews Genetics 3: 737, 2002. In this application, the term “RNAi” encompasses molecules such as short interfering RNA (siRNA), microRNAs (mRNA), small temporal RNA (siRNA). Generally speaking, RNA interference results from the interaction of double-stranded RNA with genes.

A. Antisense Oligonucleotides

The invention provides antisense oligonucleotides capable of binding CD14 messenger RNA which can inhibit polypeptide activity by targeting mRNA. Strategies for designing antisense oligonucleotides are well described in the scientific and patent literature, and the skilled artisan can design such oligonucleotides using the novel reagents of the invention. For example, gene walking/RNA mapping protocols to screen for effective antisense oligonucleotides are well known in the art, see, e.g., Ho, Methods Enzymol. 314: 168-183, 2000, describing an RNA mapping assay, which is based on standard molecular techniques to provide an easy and reliable method for potent antisense sequence selection. See also Smith, Eur. J. Pharm. Sci. 11: 191-198, 2000.

Naturally occurring nucleic acids are used as antisense oligonucleotides. The antisense oligonucleotides can be of any length; for example, in alternative aspects, the antisense oligonucleotides are between about 5 to 100, about 10 to 80, about 15 to 60, about 18 to 40. The optimal length can be determined by routine screening. The antisense oligonucleotides can be present at any concentration. The optimal concentration can be determined by routine screening. A wide variety of synthetic, non-naturally occurring nucleotide and nucleic acid analogues are known which can address this potential problem. For example, peptide nucleic acids (PNAs) containing non-ionic backbones, such as N-(2-aminoethyl)glycine units can be used. Antisense oligonucleotides having phosphorothioate linkages can also be used, as described in WO 97/03211; WO 96/39154; Mata, Toxicol Appl Pharmacol. 144: 189-197, 1997; Antisense Therapeutics, ed. Agrawal, Humana Press, Totowa, N.J., 1996. Antisense oligonucleotides having synthetic DNA backbone analogues provided by the invention can also include phosphoro-dithioate, methylphosphonate, phosphoramidate, alkyl phosphotriester, sulfamate, 3′-thioacetal, methylene(methylimino), 3′-N-carbamate, and morpholino carbamate nucleic acids, as described above.

Combinatorial chemistry methodology can be used to create vast numbers of oligonucleotides that can be rapidly screened for specific oligonucleotides that have appropriate binding affinities and specificities toward any target, such as the sense and antisense polypeptides sequences of the invention (see, e.g., Gold, J. of Biol. Chem. 270: 13581-13584, 1995).

B. siRNA

“Small interfering RNA” (siRNA) refers to double-stranded RNA molecules from about 10 to about 30 nucleotides long that are named for their ability to specifically interfere with protein expression through RNA interference (RNAi). Preferably, siRNA molecules are 12-28 nucleotides long, more preferably 15-25 nucleotides long, still more. Preferably 19-23 nucleotides long and most preferably 21-23 nucleotides long. Therefore, preferred siRNA molecules are 12, 13, 14, 15, 16, 17, 18, 19, 20, 21, 22, 23, 24, 25, 26, 27 28 or 29 nucleotides in length.

RNAi is a two-step mechanism. Elbashir et al., Genes Dev., 15: 188-200, 2001. First, long dsRNAs are cleaved by an enzyme known as Dicer in 21-23 ribonucleotide (nt) fragments, called small interfering RNAs (siRNAs). Then, siRNAs associate with a ribonuclease complex (termed RISC for RNA Induced Silencing Complex) which target this complex to complementary mRNAs. RISC then cleaves the targeted mRNAs opposite the complementary siRNA, which makes the mRNA susceptible to other RNA degradation pathways.

siRNAs of the present invention are designed to interact with a target ribonucleotide sequence, meaning they complement a target sequence sufficiently to bind to the target sequence. The present invention also includes siRNA molecules that have been chemically modified to confer increased stability against nuclease degradation, but retain the ability to bind to target nucleic acids that may be present.

C. Inhibitory Ribozymes

The invention provides ribozymes capable of binding message which can inhibit polypeptide activity by targeting mRNA, e.g., inhibition of polypeptides with CD14 activity, e.g., TLR4-signaling activity. Strategies for designing ribozymes and selecting the protein-specific antisense sequence for targeting are well described in the scientific and patent literature, and the skilled artisan can design such ribozymes using the novel reagents of the invention.

Ribozymes act by binding to a target RNA through the target RNA binding portion of a ribozyme which is held in close proximity to an enzymatic portion of the RNA that cleaves the target RNA. Thus, the ribozyme recognizes and binds a target RNA through complementary base-pairing, and once bound to the correct site, acts enzymatically to cleave and inactivate the target RNA. Cleavage of a target RNA in such a manner will destroy its ability to direct synthesis of an encoded protein if the cleavage occurs in the coding sequence. After a ribozyme has bound and cleaved its RNA target, it is typically released from that RNA and so can bind and cleave new targets repeatedly.

In some circumstances, the enzymatic nature of a ribozyme can be advantageous over other technologies, such as antisense technology (where a nucleic acid molecule simply binds to a nucleic acid target to block its transcription, translation or association with another molecule) as the effective concentration of ribozyme necessary to effect a therapeutic treatment can be lower than that of an antisense oligonucleotide. This potential advantage reflects the ability of the ribozyme to act enzymatically. Thus, a single ribozyme molecule is able to cleave many molecules of target RNA. In addition, a ribozyme is typically a highly specific inhibitor, with the specificity of inhibition depending not only on the base pairing mechanism of binding, but also on the mechanism by which the molecule inhibits the expression of the RNA to which it binds. That is, the inhibition is caused by cleavage of the RNA target and so specificity is defined as the ratio of the rate of cleavage of the targeted RNA over the rate of cleavage of non-targeted RNA. This cleavage mechanism is dependent upon factors additional to those involved in base pairing. Thus, the specificity of action of a ribozyme can be greater than that of antisense oligonucleotide binding the same RNA site.

The enzymatic ribozyme RNA molecule can be formed in a hammerhead motif, but can also be formed in the motif of a hairpin, hepatitis delta virus, group I intron or RnaseP-like RNA (in association with an RNA guide sequence). Examples of such hammerhead motifs are described by Rossi, Aids Research and Human Retroviruses 8: 183, 1992; hairpin motifs by Hampel, Biochemistry 28: 4929, 1989, and Hampel, Nuc. Acids Res. 18: 299, 1990; the hepatitis delta virus motif by Perrotta, Biochemistry 31: 16, 1992; the RnaseP motif by Guerrier-Takada, Cell 35: 849, 1983; and the group I intron by Cech U.S. Pat. No. 4,987,071. The recitation of these specific motifs is not intended to be limiting; those skilled in the art will recognize that an enzymatic RNA molecule of this invention has a specific substrate binding site complementary to one or more of the target gene RNA regions, and has nucleotide sequence within or surrounding that substrate binding site which imparts an RNA cleaving activity to the molecule.

### Methods of Treatment

Also described herein are both prophylactic and therapeutic methods of treating a subject at risk of (or susceptible to) a disorder or having a disorder associated with undesirable toll-like receptor 4 expression or activity.

### Prophylactic Methods

The invention relates to methods for preventing in a subject a disease or condition associated with an undesirable amount of toll-like receptor 4 expression or activity, by administering to the subject an agent that modulates signaling through toll-like receptor 4, TRAM/Trif, or CD14. Subjects at risk for a disorder or undesirable symptoms that are caused or contributed to by toll-like receptor 4- or CD14-mediated signaling can be identified by, for example, any of a combination of diagnostic or prognostic assays as described herein or are known in the art. In general, such disorders involve undesirable activation of the innate immune system, e.g., undesirable induction of cytokines such as TNF-α. Administration of the agent as a prophylactic agent can occur prior to the manifestation of symptoms, such that the symptoms are prevented, delayed, or diminished compared to symptoms in the absence of the agent. In some embodiments, the agent decreases binding of toll-like receptor 4 to CD14 and/or TRAM/Trif. In some embodiments, the agent decreases ligand binding to toll-like receptor 4 to CD14 and/or TRAM/Trif. The appropriate agent can be identified based on screening assays described herein. In general, such agents specifically bind to toll-like receptor 4 to CD14 and/or TRAM/Trif.

### Therapeutic Methods

Another aspect of the invention pertains to methods of TLR4, CD14 or TRAM/Trif expression or activity for therapeutic purposes. The method can include contacting a cell with an agent that modulates one or more of the activities of toll-like receptor 4 and/or CD14 activity associated with the cell, e.g., specifically binds to CD14 and inhibits signaling through toll-like receptor 4. The agent can be a compound that specifically binds to toll-like receptor 4 and selectively activates or inhibits TNF-α activity in a cell that has been induced by lipopolysaccharide, or activates or inhibits macrophage response to vesicular stomatitis virus or rabies virus. The agent can be an antibody or a protein, a naturally-occurring cognate ligand of a toll-like receptor 4 protein, a peptide, a toll-like receptor 4 or CD14 peptidomimetic, a small non-nucleic acid organic molecule, or a small inorganic molecule. These modulatory methods can be performed in vitro (e.g., by culturing the cell with the agent) or, alternatively, in vivo (e.g., by administering the agent to a subject).

The present invention provides methods of treating an individual affected by a disease or disorder characterized by undesirable expression or activity of a toll-like receptor 4 protein; for example, undesirable cytokine activity, e.g., TNF-α. In one embodiment, the method involves administering an agent (e.g., an agent identified by a screening assay described herein), or combination of agents that increases or decreases signaling through toll-like receptor 4. Conditions that can be treated by agents include those in which a subject exhibits undesirable activation of the innate immune system (e.g., undesirable inflammation).

Other disorders that can be treated by the new methods and compositions include fungal infections, sepsis, cytomegalovirus infection, tuberculosis, leprosy, bone resorption (e.g., in periodontal disease), arthritis (e.g., associated with Lyme disease), and viral hepatitis. Compounds that interfere with signaling through toll-like receptor 4 (e.g., by binding to CD14), are also useful for selectively controlling cytokine production during inflammatory reactions, e.g., those produced in response to infection by microbes such as mycobacteria.

Successful treatment of disorders related to undesirable activation of the innate immune system such as undesirable inflammation reactions can be brought about by techniques that serve to inhibit the binding of CD14 to toll-like receptor 4, or inhibit the binding of ligands to toll-like receptor 4 complexes. For example, compounds, e.g., an agent identified using an assay described herein, such as an antibody, that prove to exhibit negative modulatory activity, can be used to prevent and/or ameliorate symptoms of disorders caused by undesirable CD14 or toll-like receptor 4 activity. Such molecules can include, but are not limited to peptides, phosphopeptides, small organic or inorganic molecules, or antibodies (including, for example, polyclonal, monoclonal, humanized, anti-idiotypic, chimeric or single chain antibodies, and F_{ab}, F(_{ab′})₂ and F_{ab} expression library fragments, scFV molecules, and epitope-binding fragments thereof). In particular, antibodies and derivatives thereof (e.g., antigen-binding fragments thereof) that specifically bind to toll-like receptor 4 and can activate or inhibit TNF-α activity in a cell that has been induced by lipopolysaccharide, or activate or inhibit macrophage response to vesicular stomatitis virus or rabies virus.

### Kits

The invention provides kits comprising the compositions, e.g., nucleic acids, expression cassettes, vectors, cells, polypeptides (e.g., CD14 polypeptides, or TRAM/Trif-signal activating or toll-like receptor 4-signal activating polypeptides) and/or antibodies of the invention. The kits also can contain instructional material teaching the methodologies and uses of the invention, as described herein.

### Therapeutic Applications

The compounds and modulators identified by the methods of the present invention can be used in a variety of methods of treatment. Thus, the present invention provides compositions and methods for treating an autoimmune disease, an infectious disease, a toll-like receptor 4 signaling defect, or a CD14 cell defect.

Exemplary autoimmune diseases are acute idiopathic thrombocytopenic purpura, chronic idiopathic thrombocytopenic purpura, dermatomyositis, Sydenham's chorea, myasthenia gravis, systemic lupus erythematosus, lupus nephritis, rheumatic fever, polyglandular syndromes, bullous pemphigoid, diabetes mellitus, Henoch-Schonlein purpura, post-streptococcalnephritis, erythema nodosurn, Takayasu's arteritis, Addison's disease, rheumatoid arthritis, multiple sclerosis, sarcoidosis, ulcerative colitis, erythema multiforme, IgA nephropathy, polyarteritis nodosa, ankylosing spondylitis, Goodpasture's syndrome, thromboangitisubiterans, Sjogren's syndrome, primary biliary cirrhosis, Hashimoto's thyroiditis, thyrotoxicosis, scleroderma, chronic active hepatitis, polymyositis/dermatomyositis, polychondritis, pamphigus vulgaris, Wegener's granulomatosis, membranous nephropathy, amyotrophic lateral sclerosis, tabes dorsalis, giant cell arteritis/polymyalgia, pemiciousanemia, rapidly progressive glomerulonephritis and fibrosing alveolitis.

Exemplary infectious disease, include but are not limited to, viral or bacterial diseases. The polypeptide or polynucleotide of the present invention can be used to treat or detect infectious agents. For example, by increasing the immune response, particularly increasing the proliferation and differentiation of B and/or T cells, infectious diseases can be treated. The immune response can be increased by either enhancing an existing immune response, or by initiating a new immune response. Alternatively, the polypeptide or polynucleotide of the present invention can also directly inhibit the infectious agent, without necessarily eliciting an immune response.

Viruses are one example of an infectious agent that can cause disease or symptoms that can be treated or detected by a polynucleotide or polypeptide of the present invention. Examples of viruses, include, but are not limited to the following DNA and RNA viral families: Arbovirus, Adenoviridae, Arenaviridae, Arterivirus, Birnaviridae, Bunyaviridae, Caliciviridae, Circoviridae, Coronaviridae, Flaviviridae, Hepadnaviridae (Hepatitis), Herpesviridae (such as, Cytomegalovirus, Herpes Simplex, Herpes Zoster), Mononegavirus (e.g., Paramyxoviridae, Morbillivirus, Rhabdoviridae), Orthomyxoviridae (e.g., Influenza), Papovaviridae, Parvoviridae, Picornaviridae, Poxyiridae (such as Smallpox or Vaccinia), Reoviridae (e.g., Rotavirus), Retroviridae (HTLV-I, HTLV-II, Lentivirus), and Togaviridae (e.g., Rubivirus). Viruses falling within these families can cause a variety of diseases or symptoms, including, but not limited to: arthritis, bronchiollitis, encephalitis, eye infections (e.g., conjunctivitis, keratitis), chronic fatigue syndrome, hepatitis (A, B, C, E, Chronic Active, Delta), meningitis, opportunistic infections (e.g., AIDS), pneumonia, Burkitt's Lymphoma, chickenpox, hemorrhagic fever, Measles, Mumps, Parainfluenza, Rabies, the common cold, Polio, leukemia, Rubella, sexually transmitted diseases, skin diseases (e.g., Kaposi's, warts), and viremia. A polypeptide or polynucleotide of the present invention can be used to treat or detect any of these symptoms or diseases.

Similarly, bacterial or fungal agents that can cause disease or symptoms and that can be treated or detected by a polynucleotide or polypeptide of the present invention include, but not limited to, the following Gram-Negative and Gram-positive bacterial families and fingi: Actinomycetales (e.g., Corynebacterium, Mycobacterium, Norcardia), Aspergillosis, Bacillaceae (e.g., Anthrax, Clostridium), Bacteroidaceae, Blastomycosis, Bordetella, Borrelia, Brucellosis, Candidiasis, Campylobacter, Coccidioidomycosis, Cryptococcosis, Dermatocycoses, Enterobacteriaceae (Klebsiella, Salmonella, Serratia, Yersinia), Erysipelothrix, Helicobacter, Legionellosis, Leptospirosis, Listeria, Mycoplasmatales, Neisseriaceae (e.g., Acinetobacter, Gonorrhea, Menigococcal), Pasteurellacea Infections (e.g., Actinobacillus, Heamophilus, Pasteurella), Pseudomonas, Rickettsiaceae, Chlamydiaceae, Syphilis, and Staphylococcal. These bacterial or fungal families can cause the following diseases or symptoms, including, but not limited to: bacteremia, endocarditis, eye infections (conjunctivitis, tuberculosis, uveitis), gingivitis, opportunistic infections (e.g., AIDS related infections), paronychia, prosthesis-related infections, Reiter's Disease, respiratory tract infections, such as Whooping Cough or Empyema, sepsis, Lyme Disease, Cat-Scratch Disease, Dysentery, Paratyphoid Fever, food poisoning, Typhoid, pneumonia, Gonorrhea, meningitis, Chlamydia, Syphilis, Diphtheria, Leprosy, Paratuberculosis, Tuberculosis, Lupus, Botulism, gangrene, tetanus, impetigo, Rheumatic Fever, Scarlet Fever, sexually transmitted diseases, skin diseases (e.g., cellulitis, dermatocycoses), toxemia, urinary tract infections, wound infections. A polypeptide or polynucleotide of the present invention can be used to treat or detect any of these symptoms or diseases.

Moreover, parasitic agents causing disease or symptoms that can be treated or detected by a polynucleotide or polypeptide of the present invention include, but not limited to, the following families: Amebiasis, Babesiosis, Coccidiosis, Cryptosporidiosis, Dientamoebiasis, Dourine, Ectoparasitic, Giardiasis, Helminthiasis, Leishmaniasis, Theileriasis, Toxoplasmosis, Trypanosomiasis, and Trichomonas. These parasites can cause a variety of diseases or symptoms, including, but not limited to: Scabies, Trombiculiasis, eye infections, intestinal disease (e.g., dysentery, giardiasis), liver disease, lung disease, opportunistic infections (e.g., AIDS related), Malaria, pregnancy complications, and toxoplasmosis. A polypeptide or polynucleotide of the present invention can be used to treat or detect any of these symptoms or diseases.

Preferably, treatment using a polypeptide or polynucleotide of the present invention could either be by administering an effective amount of a polypeptide to the patient, or by removing cells from the patient, supplying the cells with a polynucleotide of the present invention, and returning the engineered cells to the patient (ex vivo therapy). Moreover, the polypeptide or polynucleotide of the present invention can be used as an antigen in a vaccine to raise an immune response against infectious disease.

### Formulation and Administration of Pharmaceutical Compositions

The invention provides pharmaceutical compositions comprising nucleic acids, peptides and polypeptides (including Abs) of the invention. As discussed above, the nucleic acids, peptides and polypeptides of the invention can be used to inhibit or activate expression of an endogenous CD14 polypeptide. Such inhibition in a cell or a non-human animal can generate a screening modality for identifying compounds to treat or ameliorate an autoimmune disease, an infectious disease, an antigen presenting cell defect or a CD14 cell defect. Administration of a pharmaceutical composition of the invention to a subject is used to generate a toleragenic immunological environment in the subject. This can be used to tolerize the subject to an antigen.

The nucleic acids, peptides and polypeptides of the invention can be combined with a pharmaceutically acceptable carrier (excipient) to form a pharmacological composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also used to stabilize or to increase or decrease the absorption of the pharmaceutical composition, including liposomal carriers. Pharmaceutically acceptable carriers and formulations for peptides and polypeptide are known to the skilled artisan and are described in detail in the scientific and patent literature, see e.g., the latest edition of Remington's Pharmaceutical Science, Mack Publishing Company, Easton, Pa. (“Remington's”).

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the peptide or polypeptide of the invention and on its particular physio-chemical characteristics.

In one aspect, a solution of nucleic acids, peptides or polypeptides of the invention are dissolved in a pharmaceutically acceptable carrier, e.g., an aqueous carrier if the composition is water-soluble. Examples of aqueous solutions that can be used in formulations for enteral, parenteral or transmucosal drug delivery include, e.g., water, saline, phosphate buffered saline, Hank's solution, Ringer's solution, dextrose/saline, glucose solutions and the like. The formulations can contain pharmaceutically acceptable auxiliary substances as required to approximate physiological conditions, such as buffering agents, tonicity adjusting agents, wetting agents, detergents and the like. Additives can also include additional active ingredients such as bactericidal agents, or stabilizers. For example, the solution can contain sodium acetate, sodium lactate, sodium chloride, potassium chloride, calcium chloride, sorbitan monolaurate or triethanolamine oleate. These compositions can be sterilized by conventional, well-known sterilization techniques, or can be sterile filtered. The resulting aqueous solutions can be packaged for use as is, or lyophilized, the lyophilized preparation being combined with a sterile aqueous solution prior to administration. The concentration of peptide in these formulations can vary widely, and will be selected primarily based on fluid volumes, viscosities, body weight and the like in accordance with the particular mode of administration selected and the patient's needs.

Solid formulations can be used for enteral (oral) administration. They can be formulated as, e.g., pills, tablets, powders or capsules. For solid compositions, conventional nontoxic solid carriers can be used which include, e.g., pharmaceutical grades of mannitol, lactose, starch, magnesium stearate, sodium saccharin, talcum, cellulose, glucose, sucrose, magnesium carbonate, and the like. For oral administration, a pharmaceutically acceptable nontoxic composition is formed by incorporating any of the normally employed excipients, such as those carriers previously listed, and generally 10% to 95% of active ingredient (e.g., peptide). A non-solid formulation can also be used for enteral administration. The carrier can be selected from various oils including those of petroleum, animal, vegetable or synthetic origin, e.g., peanut oil, soybean oil, mineral oil, sesame oil, and the like. Suitable pharmaceutical excipients include e.g., starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol.

Nucleic acids, peptides or polypeptides of the invention, when administered orally, can be protected from digestion. This can be accomplished either by complexing the nucleic acid, peptide or polypeptide with a composition to render it resistant to acidic and enzymatic hydrolysis or by packaging the nucleic acid, peptide or polypeptide in an appropriately resistant carrier such as a liposome. Means of protecting compounds from digestion are well known in the art, see, e.g., Fix, Pharm Res. 13: 1760-1764, 1996; Samanen, J. Pharm. Pharmacol. 48: 119-135, 1996; U.S. Pat. No. 5,391,377, describing lipid compositions for oral delivery of therapeutic agents (liposomal delivery is discussed in further detail, infra).

Systemic administration can also be by transmucosal or transdermal means. For transmucosal or transdermal administration, penetrants appropriate to the barrier to be permeated can be used in the formulation. Such penetrants are generally known in the art, and include, e.g., for transmucosal administration, bile salts and fusidic acid derivatives. In addition, detergents can be used to facilitate permeation. Transmucosal administration can be through nasal sprays or using suppositories. See, e.g., Sayani, Crit. Rev. Ther. Drug Carrier Syst. 13: 85-184, 1996. For topical, transdermal administration, the agents are formulated into ointments, creams, salves, powders and gels. Transdermal delivery systems can also include, e.g., patches.

The nucleic acids, peptides or polypeptides of the invention can also be administered in sustained delivery or sustained release mechanisms, which can deliver the formulation internally. For example, biodegradeable microspheres or capsules or other biodegradeable polymer configurations capable of sustained delivery of a peptide can be included in the formulations of the invention (see, e.g., Putney, Nat. Biotechnol. 16: 153-157, 1998).

For inhalation, the nucleic acids, peptides or polypeptides of the invention can be delivered using any system known in the art, including dry powder aerosols, liquids delivery systems, air jet nebulizers, propellant systems, and the like. See, e.g., Patton, Biotechniques 16: 141-143, 1998; product and inhalation delivery systems for polypeptide macromolecules by, e.g., Dura Pharmaceuticals (San Diego, Calif.), Aradigrn (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, e.g., air jet nebulizers.

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes, see below), carbohydrates, or synthetic polymers (discussed above). For a general discussion of pharmacokinetics, see, e.g., Remington's, Chapters 37-39.

The nucleic acids, peptides or polypeptides of the invention can be delivered alone or as pharmaceutical compositions by any means known in the art, e.g., systemically, regionally, or locally (e.g., directly into, or directed to, a tumor); by intraarterial, intrathecal (IT), intravenous (IV), parenteral, intra-pleural cavity, topical, oral, or local administration, as subcutaneous, intra-tracheal (e.g., by aerosol) or transmucosal (e.g., buccal, bladder, vaginal, uterine, rectal, nasal mucosa). Actual methods for preparing administrable compositions will be known or apparent to those skilled in the art and are described in detail in the scientific and patent literature, see e.g., Remington's. For a “regional effect,” e.g., to focus on a specific organ, one mode of administration includes intra-arterial or intrathecal (IT) injections, e.g., to focus on a specific organ, e.g., brain and CNS (see e.g., Gurun, Anesth Analg. 85: 317-323, 1997). For example, intra-carotid artery injection if preferred where it is desired to deliver a nucleic acid, peptide or polypeptide of the invention directly to the brain. Parenteral administration is a preferred route of delivery if a high systemic dosage is needed. Actual methods for preparing parenterally administrable compositions will be known or apparent to those skilled in the art and are described in detail, in e.g., Remington's, See also, Bai, J. Neuroimmunol. 80: 65-75, 1997; Warren, J. Neurol. Sci. 152: 31-38, 1997; Tonegawa, J. Exp. Med. 186: 507-515, 1997.

In one aspect, the pharmaceutical formulations comprising nucleic acids, peptides or polypeptides of the invention are incorporated in lipid monolayers or bilayers, e.g., liposomes, see, e.g., U.S. Pat. Nos. 6,110,490; 6,096,716; 5,283,185; 5,279,833. The invention also provides formulations in which water soluble nucleic acids, peptides or polypeptides of the invention have been attached to the surface of the monolayer or bilayer. For example, peptides can be attached to hydrazide-PEG-(distearoylphosphatidyl) ethanolamine-containing liposomes (see, e.g., Zalipsky, Bioconjug. Chem. 6: 705-708, 1995). Liposomes or any form of lipid membrane, such as planar lipid membranes or the cell membrane of an intact cell, e.g., a red blood cell, can be used. Liposomal formulations can be by any means, including administration intravenously, transdermally (see, e.g., Vutla, J. Pharm. Sci. 85: 5-8, 1996), transmucosally, or orally. The invention also provides pharmaceutical preparations in which the nucleic acid, peptides and/or polypeptides of the invention are incorporated within micelles and/or liposomes (see, e.g., Suntres, J. Pharm. Pharmacol. 46: 23-28, 1994; Woodle, Pharm. Res. 9: 260-265, 1992). Liposomes and liposomal formulations can be prepared according to standard methods and are also well known in the art, see, e.g., Remington's; Akimaru, Cytokines Mol. Ther. 1: 197-210, 1995; Alving, Immunol. Rev. 145: 5-31, 1995; Szoka, Ann. Rev. Biophys. Bioeng. 9: 467, 1980, U.S. Pat. Nos. 4, 235,871, 4,501,728 and 4,837,028.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects can be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any compound used in the method of the invention, the therapeutically effective dose can be estimated initially from cell culture assays. A dose can be formulated in animal models, e.g., of inflammation or disorders involving undesirable inflammation, to achieve a circulating plasma concentration range that includes the IC₅₀ (i.e., the concentration of the test compound which achieves a half-maximal inhibition of symptoms) as determined in cell culture. Such information can be used to more accurately determine useful doses in humans. Levels in plasma can be measured, for example, by high performance liquid chromatography, generally of a labeled agent. Animal models useful in studies, e.g., preclinical protocols, are known in the art, for example, animal models for inflammatory disorders such as those described in Sonderstrup (Springer, Sem. Immunopathol. 25: 35-45, 2003) and Nikula et al., Inhal. Toxicol. 4(12): 123-53, 2000), and those known in the art, e.g., for fungal infection, sepsis, cytomegalovirus infection, tuberculosis, leprosy, viral hepatitis, and infection (e.g., by mycobacteria).

As defined herein, a therapeutically effective amount of protein or polypeptide such as an antibody (i.e., an effective dosage) ranges from about 0.001 to 30 mg/kg body weight, for example, about 0.01 to 25 mg/kg body weight, about 0.1 to 20 mg/kg body weight, or about 1 to 10 mg/kg, 2 to 9 mg/kg, 3 to 8 mg/kg, 4 to 7 mg/kg, or 5 to 6 mg/kg body weight. The protein or polypeptide can be administered one or several times per day or per week for between about 1 to 10 weeks, for example, between 2 to 8 weeks, between about 3 to 7 weeks, or about 4, 5, or 6 weeks. In some instances the dosage can be required over several months or more. The skilled artisan will appreciate that certain factors can influence the dosage and timing required to effectively treat a subject, including, but not limited to the severity of the disease or disorder, previous treatments, the general health and/or age of the subject, and other diseases present. Moreover, treatment of a subject with a therapeutically effective amount of an agent such as a protein or polypeptide (including an antibody) can include a single treatment or, preferably, can include a series of treatments.

For antibodies, the dosage is generally 0.1 mg/kg of body weight (for example, 10 mg/kg to 20 mg/kg). Partially human antibodies and fully human antibodies generally have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al., J. Acquired Immune Deficiency Syndromes and Human Retrovirology, 14: 193, 1997).

The present invention encompasses agents or compounds that modulate expression or activity of TNF-α by modulating signaling through toll-like receptor 4 or CD14. An agent can, for example, be a small molecule. Such small molecules include, but are not limited to, peptides, peptidomimetics (e.g., peptoids), amino acids, amino acid analogs, small non-nucleic acid organic compounds or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid of the invention, a physician, veterinarian, or researcher can, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

An antibody or fragment thereof can be linked, e.g., covalently and/or with a linker to another therapeutic moiety such as a therapeutic agent or a radioactive metal ion, to form a conjugate. Therapeutic agents include, but are not limited to, antibiotics (e.g., dactinomycin (formerly actinomycin), bleomycin, mithramycin, and anthramycin (AMC)).

The conjugates described herein can be used for modifying a given biological response. For example, the moiety bound to the antibody can be a protein or polypeptide possessing a desired biological activity. Such proteins can include, for example, a toxin such as abrin, ricin A, Pseudomonas exotoxin, or diphtheria toxin; a protein such as tumor necrosis factor, .alpha.-interferon, .beta.-interferon, nerve growth factor, platelet derived growth factor, tissue plasminogen activator; or, biological response modifiers.

Alternatively, an antibody can be conjugated to a second antibody to form an antibody heteroconjugate as described by Segal in U.S. Pat. No. 4,676,980.

The pharmaceutical compositions can be included in a container, pack, or dispenser together with instructions for administration.

Compounds as described herein can be used for the preparation of a medicament for use in any of the methods of treatment described herein.

The pharmaceutical compositions are generally formulated as sterile, substantially isotonic and in full compliance with all Good Manufacturing Practice (GMP) regulations of the U.S. Food and Drug Administration.

### Treatment Regimens: Pharmacokinetics

The pharmaceutical compositions of the invention can be administered in a variety of unit dosage forms depending upon the method of administration. Dosages for typical nucleic acid, peptide and polypeptide pharmaceutical compositions are well known to those of skill in the art. Such dosages are typically advisorial in nature and are adjusted depending on the particular therapeutic context, patient tolerance, etc. The amount of nucleic acid, peptide or polypeptide adequate to accomplish this is defined as a “therapeutically effective dose.” The dosage schedule and amounts effective for this use, i.e., the “dosing regimen,” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e., the pharmaceutical composition's rate of absorption, bioavailability, metabolism, clearance, and the like. See, e.g., the latest Remington's; Egleton, Peptides 18: 1431-1439, 1997; Langer, Science 249: 1527-1533, 1990.

In therapeutic applications, compositions are administered to a patient suffering from autoimmune disease, an infectious disease, an antigen presenting cell defect or a CD4 cell defect in an amount sufficient to at least partially arrest the condition or a disease and/or its complications. For example, in one aspect, a soluble peptide pharmaceutical composition dosage for intravenous (IV) administration would be about 0.01 mg/hr to about 1.0 mg/hr administered over several hours (typically 1, 3, or 6 hours), which can be repeated for weeks with intermittent cycles. Considerably higher dosages (e.g., ranging up to about 10 mg/ml) can be used, particularly when the drug is administered to a secluded site and not into the blood stream, such as into a body cavity or into a lumen of an organ, e.g., the cerebrospinal fluid (CSF).

The following Examples of specific embodiments for carrying out the present invention are offered for illustrative purposes only, and are not intended to limit the scope of the present invention in any way.

The disclosures of all publications, patents and patent applications cited herein are hereby incorporated by reference in their entirety.

### Exemplary Embodiments

### EXAMPLE 1

The Heedless Mutation

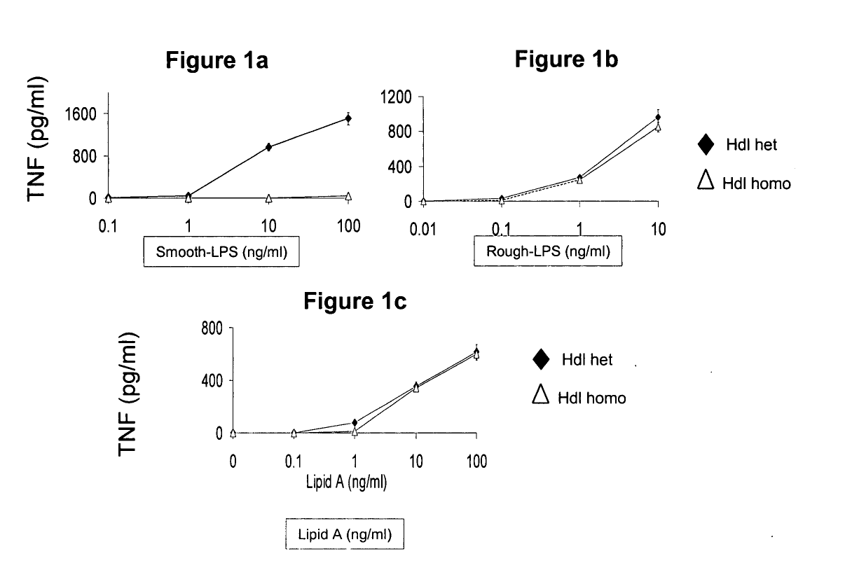

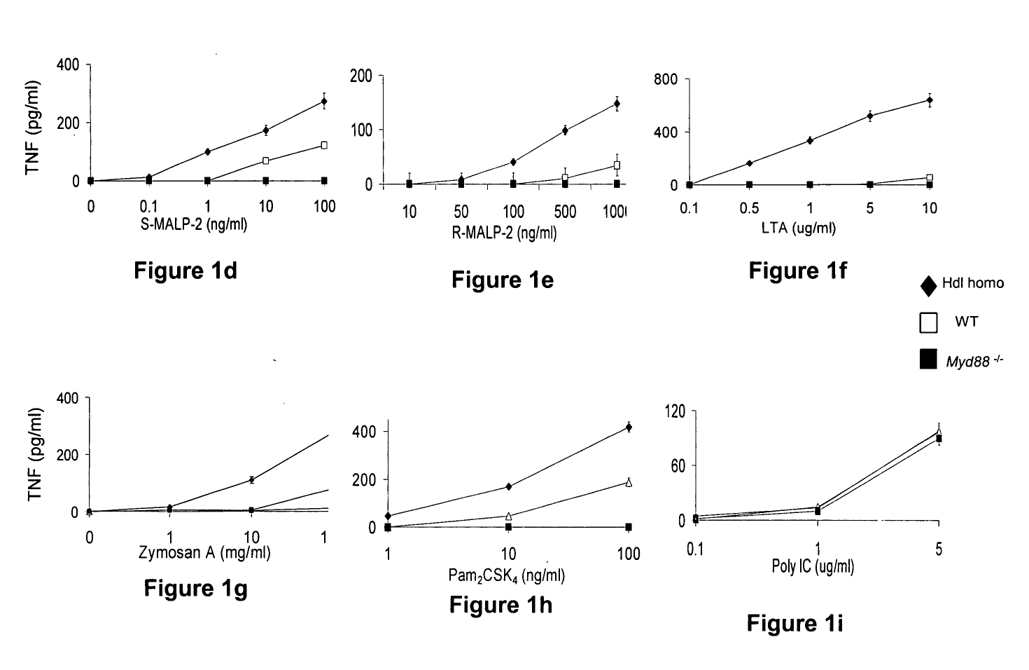

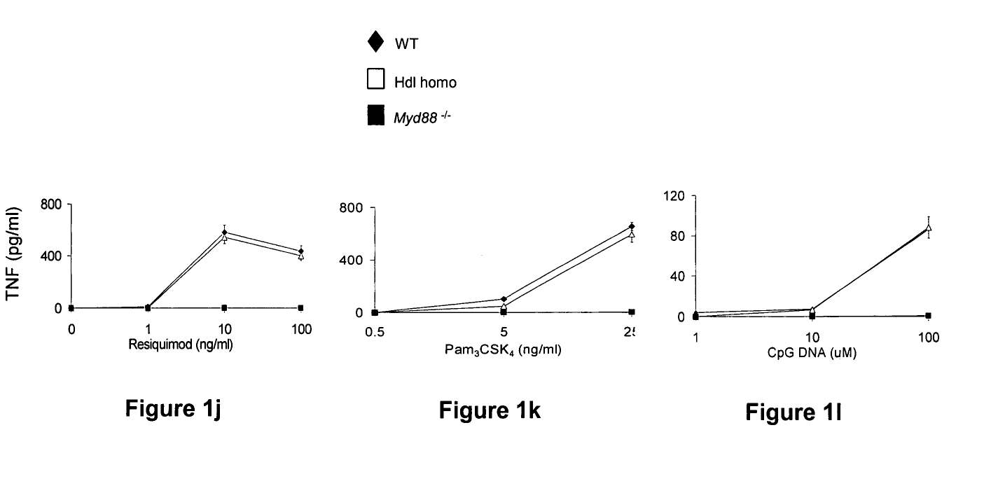

‘Heedless’, a transmissible recessive LPS-hyporesponsive phenotype identified in a G3 animal, was bred to produce a homozygous stock. The mutation was found to prevent TNF production in response to smooth LPS chemotypes, but not rough LPS chemotype or lipid A from Salmonella. minnesota (FIG. 1a-c). The mutation also produced partial impairment of the response to TLR2-TLR6 ligands, including synthetic di-acylated macrophage activating lipopeptide-2 (MALP-2; impairment of R stereoisomer>S stereoisomer) and Pam₂CSK₄, as well as highly purified lipoteichoic acid, and zymosan A (FIG. 1d-h). The response to Pam₃CSK₄, a TLR2-TLR1 ligand, and to other known TLR ligands, such as Resiquimod (TLR7), poly IC (TLR3) and CpG (TLR9), was normal (FIG. 1i-l). Hence, the mutation exerted a ligand-restricted but essentially complete effect on signaling via TLR4, and a broad but partial effect on signaling via the TLR2-TLR6 complex.

FIG. 1 shows rough LPS and TLR2-6 specificity of the Heedless mutation. Wild-type (WT), heterozygous Heedless (Hdl het), homozygous Heedless (Hdl homo) or Myd88-deficient mice were injected intraperitoneally with 3% thioglycolate to induce macrophage infiltration. Macrophages were isolated, cultured and dose-response experiments were performed for each specific inducer as indicated. After 4 h of incubation with the inducer at 37° C., supernatants were collected and assayed in duplicate for TNF concentrations using the L929 bioassay as described previously. Values represent mean±SEM (n=6 mice of greater). The inducers used are smooth LPS (a), rough LPS (b), Lipid A (c), S-MALP-2 (d), R-MALP-2 (e), LTA (f), Zymosan A (g), Pam₂CSK₄ (h), Poly I:C (i), Resiquimod (), Pam₃CSK₄ (k) and CpG-containing DNA (l). Similar results were observed in three independent experiments.

### EXAMPLE 2

Resistance to Shock Independent of LPS Chemotype

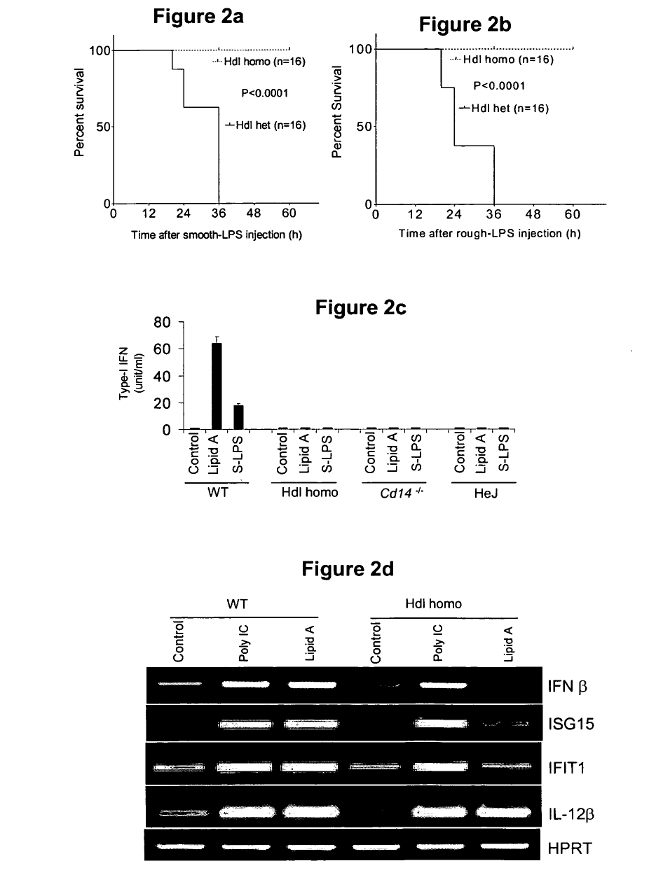

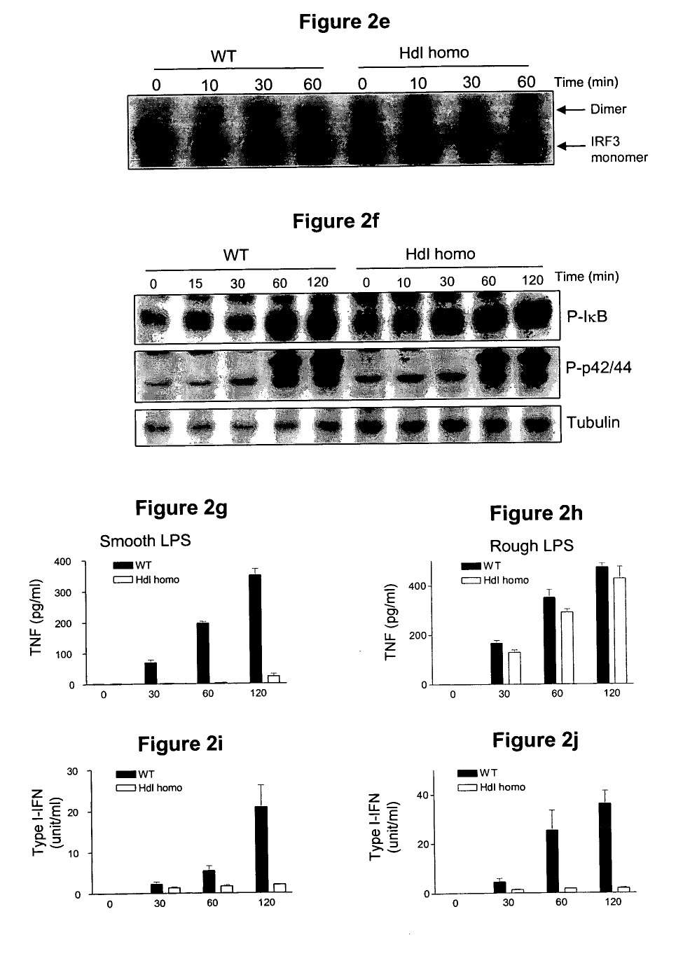

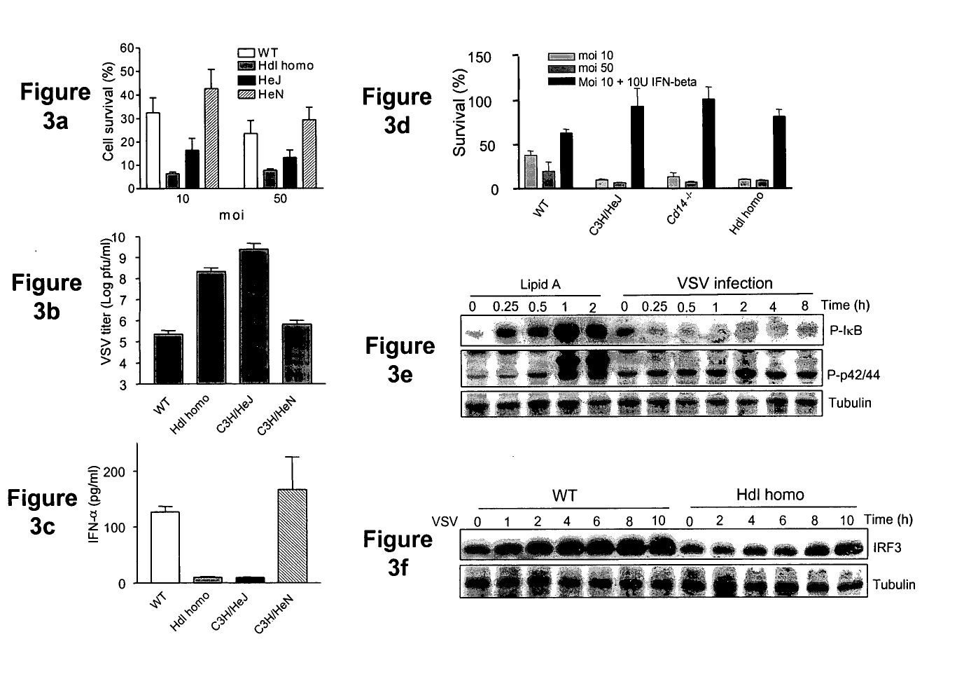

Because Heedless selectively prevented TNF production in response to smooth LPS, it was anticipated that the mutation would only protect mice against the lethal effect of smooth (but not rough) LPS. Mice homozygous for the mutation, or heterozygous C57BL/6 littermates, were injected with 1 mg of LPS (either rough or smooth chemotype) by an intraperitoneal route. All heterozygous mice receiving either rough or smooth LPS died within 36 hours. Contrary to expectation, all homozygous Heedless mice survived, whether rough or smooth LPS was administered. Subjectively, all Heedless homozygotes showed far less sensitivity to both rough and smooth chemotypes than the controls (FIGS. 2a and 2b). Although rough LPS can induce Heedless macrophages to produce TNF, the mutation must forbid at least some aspects of the LPS response.

### EXAMPLE 3

Heedless Blocks LPS-induced Type I IFN Production

All forms of LPS signal via TLR4 through a MyD88-dependent pathway involving the adapters MyD88 and Mal, and a MyD88-independent pathway involving the adapters TRIF and TRAM. Poltorak et al., Science 282: 2085-2088, 1998; Hoebe et al., Nature 424: 743-748, 2003; Yamamoto et al., Science 301: 640-643, 2003; Yamamoto et al., Nat. Immunol. 4: 1144-1150, 2003; Kawai et al., 11(1): 115-122, 1999; Yamamoto et al., Nature 420: 324-329, 2002; Horng et al., Nature 420: 329-333, 2002. LPS-induced IFN-β production is entirely dependent upon TRWF and TRAM, which permit the phosphorylation and dimerization of the INF-β transcription factor IRF-3. Hoebe et al., Nature 424: 743-748, 2003; Yamamoto et al., Science 301: 640-643, 2003; Yamamoto et al., Nat. Immunol. 4: 1144-1150, 2003. INF-β augments its own synthesis via autoamplification loops utilizing the tyrosine kinase Tyk2 and the signal transducer and activator of transcription STAT-1, and is known to play a major role in LPS toxicity. Karaghiosoff et al., Nat. Immunol. 4: 471-477, 2003. The effect of the Heedless mutation on the production of type I IFN was examined, and it was found that the mutation prevented both smooth LPS and lipid A from signaling via the MyD88-independent pathway (FIG. 2c, d). Specifically, Heedless prevented the production of type I WFN and IFN-β mRNA, as well as the induction of IFN-inducible genes such as IFIT1, ISG15. In response to lipid A, the formation of the IRF-3 phosphodimer was not detected in heedless mutant cells. (FIG. 2e). However, activation of the transcription factor NF-κB and phosphorylation of the mitogen activated protein kinases ERK1 and ERK2 occurred normally in Heedless macrophages (FIG. 2f). The production of TNF and type I IFN in vivo in mice injected with either rough LPS or smooth LPS was also examined. Both smooth and rough LPS induced robust production of TNF and type I IFNs in sera of wild-type mice (FIG. 2g-j), whereas smooth LPS did not induce TNF or type I IFN in the sera of Heedless mutant mice (FIG. 2g, 2i). Rough LPS induced TNF production in both Heedless and wild-type mice, but did not stimulate type I IFN production in Heedless mice (FIG. 2h, j). These results agree with analyses of macrophage responses ex vivo, and are consistent with the hypothesis that Heedless inhibits lethality by preventing LPS-induced Type I IFN production in vivo. Thus, the LPS receptor complex can either selectively initiate MyD88-dependent signaling or initiate both MyD88-dependent and MyD88-independent signaling. The protein affected by Heedless governs the availability of the MyD88-independent pathway and the Heedless protein is necessary for smooth LPS to induce any form of TLR4 signaling. Finally, the Heedless protein is necessary for lipid A (or rough LPS) to induce signaling via the MyD88-independent pathway, but is unnecessary for lipid A (or rough LPS) to induce signaling via the MyD88-dependent pathway.

FIG. 2 shows Heedless prevents INF-β induction by LPS. Age-matched male Heedless heterozygotes and homozygotes (litter-mates) were injected intraperitoneally with 1 mg of LPS from S. abortus (smooth LPS) (a) or S. minnesota Re595 (rough LPS) (b). Survival was monitored over a period of three days, and the data are expressed as a Kaplan-Meier plot (P<0.0001). (c) Type I IFN activity measured in the supernatant of macrophages from indicated mutant mice (n=6). Cells were treated with smooth LPS (100 ng/ml) or lipid A (100 ng/ml) for 4 hours. Error bars represent SEM of determinations. (d) Peritoneal macrophages from wild-type C57BLU6 or Heedless mice were treated with Poly IC or lipid A for 2 hours. Total RNA was isolated from cells and induction of IFNβ, IFN-inducible gene IFIT1, ISG15, and IL-12β, HPRT were analyzed by RT-PCR. Immunoblot analysis of IRF-3 activation (e) or IκB and ERK (p42/44) phosphorylation (f) in wild-type and Heedless macrophages treated with lipid A (100 ng/ml). (g-j) The production of TNF and type I interferon in the serum of mice injected with LPS. Age-matched (8 weeks old) wild-type and Heedless mice (4 mice per group) were injected intraperitoneally with 0.5 mg of either smooth LPS from S. abortus (g, i) or rough LPS from S. minnesota Re595 (h, j), blood was collected at the indicated times and the concentration of TNF (g, h) and type I IFN (i, j) in serum was analyzed by TNF or IFN bioassay as described in the materials and methods. Similar results were obtained in an additional experiment.

### EXAMPLE 4

VSV Signals via Heedless and TLR4

The key role of type I interferon in the restriction of viral proliferation led to an examination of the requirement for Heedless in the control of vesicular stomatitis virus (VSV) growth in macrophages ex vivo. Heedless macrophages were far more susceptible to lysis than wild-type C57BL/6 macrophages when exposed to VSV at a MOI ranging between 10 and 50 (FIG. 3a). Moreover, LPS-unresponsive Tlr4^{Lps-d/Lps-d} macrophages from C3H/HeJ mice were markedly hypersusceptible compared to LPS-responsive Tlr4^{Lps-n/Lps-n} macrophages from C3H/HeN mice. At least 1000 times more virus was found in Heedless macrophages as compared with infected wildtype cells, suggesting that macrophages from Heedless mice were not able to contain the infection (FIG. 3b). In addition, the production of IFN-α by Heedless macrophages was profoundly reduced (FIG. 3c). The lytic effect of VSV on Heedless or Tlr4^{Lps-d/Lps-d} macrophages was prevented by treating the cells with type I interferon before infection (FIG. 3d).

To exclude any possibility that LPS contamination of the VSV inoculum was responsible for the Heedless- and TLR4-dependent resistance to infection, the virus was serially passaged on Vero cell monolayers using the same medium as that used for macrophage culture. Viral infection was performed by directly applying diluted medium from the producer line to the macrophage monolayers, without any effort at viral purification or concentration (mock-infected producer cells were used as controls), and viral titer was measured separately. Unlike LPS, the virus was unable to induce a TNF response 8h, 16h, 21h, and 36h after infection, and no NF-κB activation nor ERK phosphorylation was detected (FIG. 3e). Although VSV infection of macrophages from C57BU6 mice induced a strong IRF-3 activation response (shown as the shifted bands) after 4 h (FIG. 3f), infection of macrophages from Heedless mice induced minimal IRF-3 activation after 10 h. Hence, the Heedless-TLR4-IRF-3-IFN-β axis is required for a protective response to VSV. No enhancement of susceptibility was noted in macrophages obtained from TLR3-deficient mice.

FIG. 3 shows Heedless macrophages are hypersensitive to cytolysis induced by VSV. The cytolytic effect of VSV was examined in thioglycolate-elicited peritoneal macrophages from C57BL/6 (WT), heedless (Hdl), C3H/HeN (HeN) and Tlr4^{Lps-d/Lps-d} C3H/HeJ (HeJ) mice. Cell survival (a), viral titer (b), and IFN-α in culture medium (c) were measured 48 h after the infection which was initiated with a multiplicity of infection (moi) of 10 or 50 viral particles per macrophage. Cell survival was also determined in cultures pretreated with IFN-β was 4 h before viral infection (d). Immunoblot analysis of IκB, ERK (p42/44) phosphorylation in wild-type macrophages infected with VSV (50 moi) for indicated times (e). Immunoblot analysis of IRF-3 activation in wild-type and Heedless macrophages infected with VSV (50 moi) for indicated times (f). Similar results were observed in three independent experiments.

### EXAMPLE 5

Heedless Corresponds to a Cd14 Mutation

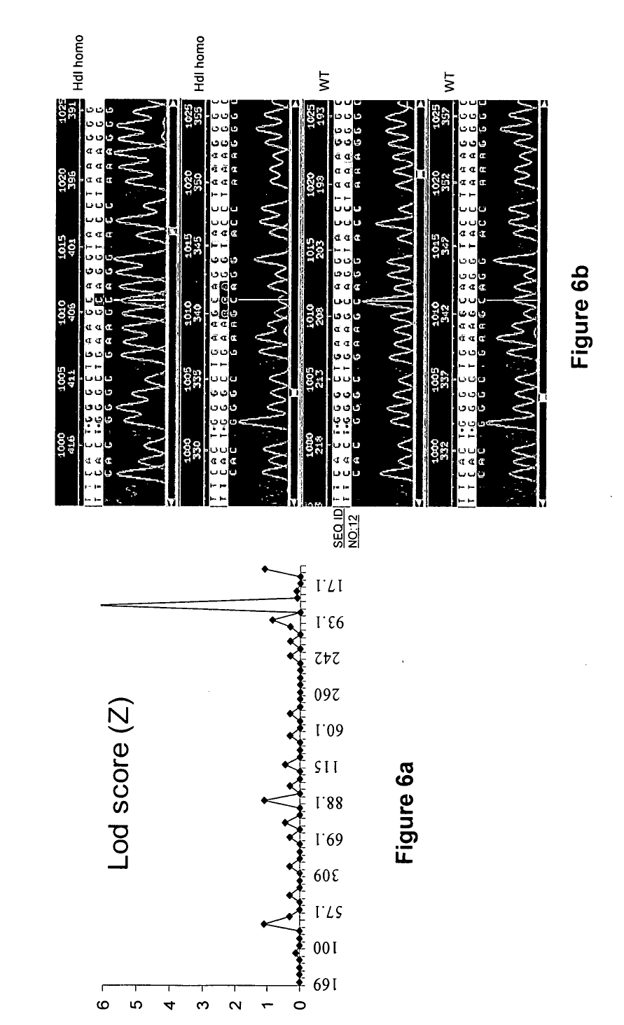

The Heedless mutation was mapped to central mouse chromosome 18, whereby >98.2% of the mouse genome was excluded on 24 meioses (FIG. 6). The critical region contained the gene encoding CD14. When the CD14 cDNA was amplified by RT-PCR and sequenced, a premature stop codon (Q284X) was observed in the Heedless sample. Ferrero and Goyert, Nucleic Acids Res., 16: 4173, 1988; NCBI GenBank P08571.

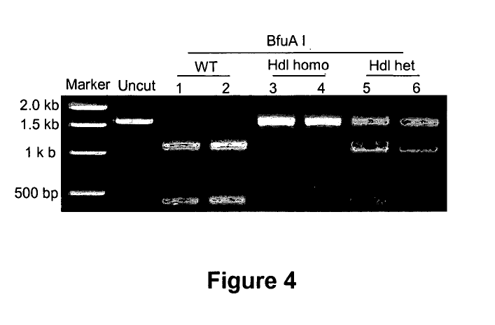

Because the morphology of the critical base was unusual, presenting the appearance of a double peak in both strands of DNA from numerous homozygotes, the existence of the mutation was confirmed by restriction endonuclease cleavage using the enzyme BfuA-I, which is capable of cutting the wild-type allele, but not the Heedless allele (FIG. 4). The mutation predicted the removal of 83 carboxy-terminal amino acids, which form the second leucine-rich repeat LRR domain of the 366 amino acid CD14 polypeptide chain.

FIG. 4 shows Heedless, a mutation in Cd14, detected by restriction endonuclease cleavage. A fragment of the Cd14 gene, containing the hdl mutation site, was amplified by PCR from wild-type mice, Heedless homozygotes and heterozygotes using genomic DNA template. About 0.2 microgram of each fragment was digested using restriction enzyme BfuAI at 50° C. for 2 hours and separated on a 1% agarose gel. The uncut PCR fragment is 1571 bp, after BfuA I digestion. Digestion of the wild-type (but not the hdl) sequence is predicted to yield a fragment 1111 bp in length and another fragment 460 bp in length.

FIG. 6 shows the Heedless mutation, mapped and identified by sequencing. (a). Phenotypic classification of F2 mice was based on measurement of LPS-induced macrophage TNF production, using the L929 bioassay. On 24 meioses, confinement of hdl to the central region of chromosome 18 was achieved with a peak LOD score>6. (b). Consed display of the mutation, showing sequence from the distal coding region of an hdl/hdl homozygote (top traces; bidirectional sequencing) and from a normal C57BL/6 mouse (bottom traces). A C is replaced by T in the mutant strain, but appears as a double peak despite homozygosity.

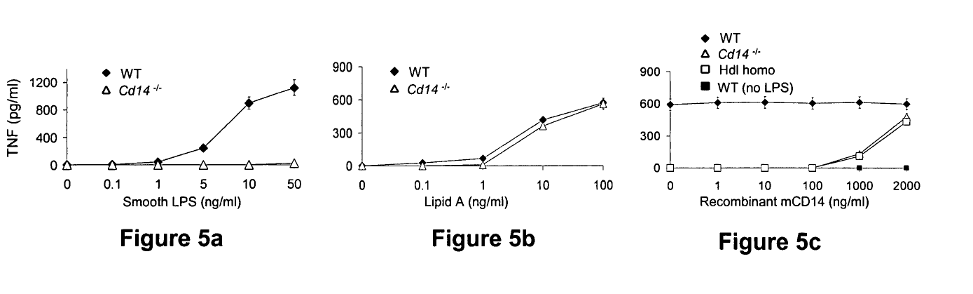

Because it has not previously been reported that CD14 exercises selectivity in concentrating the LPS response, it was decided to exclude the possibility that an unrelated mutation might have caused the phenotype that was observed, and Cd14^{-/-} mice were examined. Macrophages from these animals showed precisely the same phenotype as that observed in Heedless cells (FIGS. 2c, 3d, 5a, and 5b). Moreover, when added to macrophage cultures at a high concentration (greater than 2 μg/ml), purified recombinant soluble CD14 was capable of rescuing the heedless phenotype (FIG. 5c). Thus, the Heedless phenotype is caused by a functionally null allele of CD14.

FIG. 5 shows rescue of smooth LPS responsiveness in Cd14 homozygous mutant cells by recombinant mCD14. a, b. Peritoneal macrophages from normal or CD14 knock-out mice were treated with the indicated amounts of smooth LPS (a), lipid A (b), or 50 ng/ml smooth LPS plus indicated amount of recombinant mCD14 (c) for 4 hours. In (c), both the response of Cd14^{-/-} cells and Hdl mutant cells is shown. TNF production was measured as the endpoint of response.

### EXAMPLE 6

CD14 Required for LPS-induced Activation of the TRIF-TRAM Pathway and for VSV Response

Unbiased phenotypic screening and positional cloning reveal that CD14 serves a different function than that which was previously ascribed to it. Rather than simply concentrating the LPS signal, CD14 was absolutely required for LPS-induced activation of the TRWF-TRAM pathway. It was also essential for the response to VSV, which entailed exclusive TLR4-mediated activation of IRF-3 phosphodimer formation. To a lesser extent, CD14 also participated in signaling via the TLR2-TLR6 receptor complex (also known to incorporate CD36).

From the foregoing presentation, it might be supposed that CD14 can distinguish between rough and smooth LPS chemotypes. However, the data do not permit this conclusion. On the contrary, because the TLR4-MD-2 complex makes a distinction between smooth and rough LPS in the absence of CD14, it is the TLR4-MD-2 complex that has discriminatory ability, whereas CD14 enables specific biological activities of both rough and smooth LPS chemotypes. CD14 imparts an ability to trigger MyD88-independent signaling in response to rough LPS or lipid A. In contrast, CD14 imparts all TLR4-dependent signaling activity in response to smooth LPS. Thus, CD14 does not discriminate between rough and smooth chemotypes, but acts as an essential factor in signaling by both.

The LPS receptor behaves as a switch with two stops; either “full activation” of the receptor or restricted MyD88-dependent activation can occur, depending upon the presence or absence of CD14, and the activating ligand. Rough LPS or lipid A stimulate MyD88-dependent activation in the absence of CD14, which is consistent with the hypothesis that lipid A molecules undergo direct contact with TLR4 in order to signal. Poltorak et al., Proc. Natl. Acad. Sci. USA 97: 2163-2167, 2000; Lien et al., J. Clin. Invest. 105: 497-504, 2000. Because some cells express TLR4-MD-2 but not CD14, strictly MyD88-dependent signaling initiated by rough LPS is likely to occur when animals are infected with organisms that produce the rough LPS chemotype, and is not a phenomenon restricted to CD14-deficient mice (e.g., mast cells and B cells do not express CD14; M. Huber, et al., personal communication). Although previously considered to signal by way of TLR7, VSV clearly depends on the CD14-TLR4 pathway in macrophages, and elicits IRF-3-dependent production of IFN-β, but does not activate the MyD88 pathway. Lund et al., Proc. Natl. Acad. Sci U.S.A. 101: 5598-5603, 2004.

One hypothesis that would account for our observations holds that CD14 permits MyD88-independent signal transduction through an effect on supramolecular structure of the TLR4-MD-2 complex (i.e., by means of induced proximity of complexes), whereas MyD88-dependent signaling can result from direct stimulation of disordered TLR4-MD-2 complexes by rough LPS or lipid A. In an alternative model, CD14 might allow the TLR4-MD-2 complex to undergo a conformational change that permits MyD88-independent signaling when LPS is present. In either model, it is envisioned that CD14 directly engages both rough and smooth chemotype LPS molecules; the TLR4-MD-2 is able to engage only the former unassisted. The three-dimensional structure of CD14, recently determined by X-ray crystallography, has disclosed the likely binding site for LPS and other microbial ligands, as well as sites that may be involved in downstream signal transduction, and may ultimately contribute to the interpretation of the effects reported here. Kim et al., J. Biol. Chem. 280: 11347-11351,2005.

It should be noted that mice homozygous for a Cd14 null allele vs. a Trif null allele are phenotypically distinguishable from one another. In both cases, there is failure to respond to LPS with INF-β production. However, Trif mutant macrophages also show rather severe impairment of lipid A-induced TNF production whereas Cd14 null macrophages are perfectly able to produce TNF in response to lipid A. Hoebe et al., Nature 424: 743-748, 2003. This is probably due to functionally important interactions between TRIF and components of the MyD88-dependent pathway; e.g., TRAF-6. Jiang et al., Proc. Natl. Acad. Sci U. S. A 101: 3533-3538, 2004. Moreover, Cd14 mutations affect TLR2-TLR6 sensing, whereas Trif mutations do not. Hoebe et al., Nature 424: 743-748, 2003; Yamamoto et al., Science 301: 640-643, 2003.

In its dual role as a facilitator of TLR2-TLR6 and TLR4 stimuli (including LPS and a yet-unknown product of VSV infection), CD14 transduces signals from structurally disparate molecules, and it can be inferred that the TLR2-TLR6 complex interacts with CD14 much as the TLR4-MD-2 complex does. Both LTA and MALP-2 (but not zymosan) partially depend upon CD36 as well as CD14 to signal via the TLR2-TLR6 heterodimer, and the phenotype of compound homozygotes for a CD36 null allele (Cd36^{obl}) and Cd14^{Hdl} is presently being examined. Hoebe et al., Nature 433: 523-527, 2005. The essential function of the CD14-TLR4 signaling axis in macrophage resistance to VSV infection was unexpected, and it is clear that at least in macrophages TLR4, rather than TLR3 or TLR7, is of key importance to the detection of this microbe. It is possible that different cell types sense the same virus via different TLRs by recognizing specific viral product. The identity of the molecule that activates CD14 and permits a MyD88-independent TLR4 response in the course of VSV infection remains to be determined.

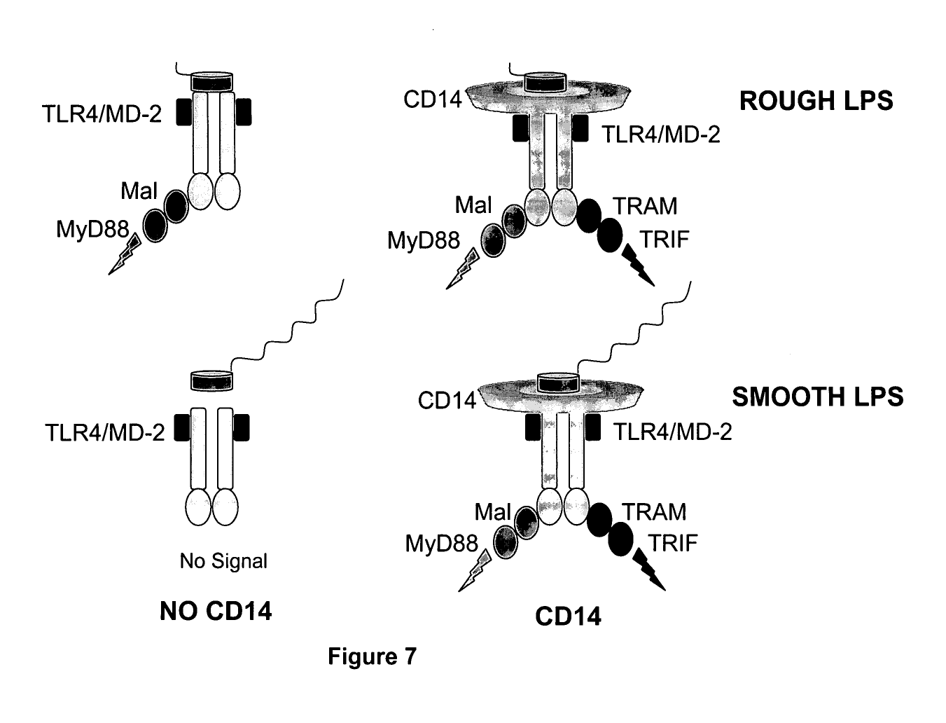

FIG. 7 shows a schematic illustration summarizing the interactions between rough and smooth LPS (a lipid A “cylinder” with differing length polysaccharide [wavy line]), the TLR4/MD-2 complex (rectangles of blue and black color, respectively), and CD14 (toroid). CD14 permits qualitatively equal responses to smooth and rough LPS. Rough LPS can activate MyD88-independent signaling in the absence of CD14. Smooth LPS can activate no LPS signals in the absence of CD14.

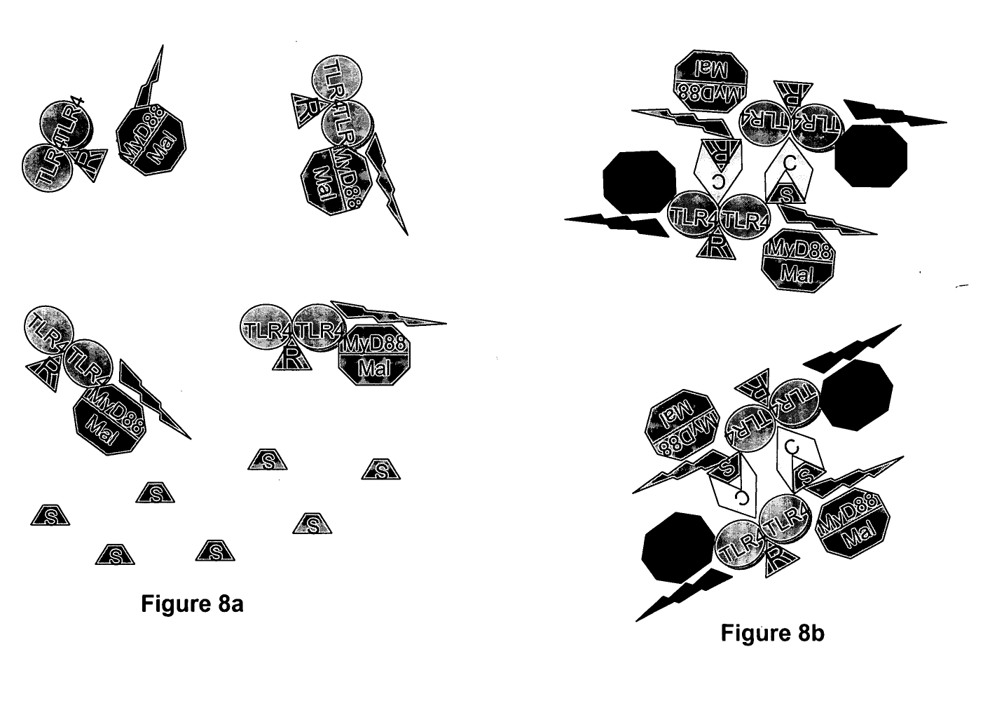

FIG. 8 shows a hypothetical mechanism whereby CD14 can permit MyD88-independent signaling from the TLR4 complex. A top view of smooth and rough LPS, CD14, TLR4/MD-2, and adapter proteins is represented (the cell membrane is transparent). A. In the absence of CD14, rough LPS can stimulate MyD88/Mal recruitment from individual TLR4/MD-2 complexes, but smooth LPS is excluded from interaction. B. In the presence of CD14, a supramolecular aggregation between TLR4/MD-2 complexes occurs, and TRIF/TRAM recruitment occurs as a result of induced proximity. Both smooth and rough LPS molecules can be engaged by CD14, and are incorporated into the assembly complex.

### EXAMPLE 7

Methodology

Mice. C57BLU6 mice were used in mutagenesis as previously described. Hoebe et al., Nature 424: 743-748, 2003. Thioglycolate (TG)-elicited peritoneal macrophages were harvested three days after TG injection and screened for responses to TLR agonists as previously described. Hoebe et al., Nature 424: 743-748, 2003. Cd14^{-/-} and C3H/HeJ mice were obtained from the Jackson Laboratories. C3H/HeN mice were obtained from Charles River. All experiments were carried out in compliance with the rules of the TSRI Animal Use Committee.

Genetic mapping and positional identification of Heedless. Heedless homozygotes were outcrossed to C3H/HeN mice and backcrossing to the Heedless stock. 24 mice were genotyped at sixty informative microsatellite loci. The mutation was confined between the two chromosome 18 markers (separated from the proximal marker by a single crossover, and from the distal marker by numerous crossovers). Genotyping was performed by fragment length analysis using fluorescent primers and an ABI 3100 DNA sequencer. Sequence analysis was also performed with this machine, and in all instances was performed on uncloned DNA fragments using internal primers.

Reagents. Salmonella minnesota Re595 (rough) LPS, Salmonella abortus equi (smooth) LPS, Salmonella typhimurium (smooth) LPS, Lipid A from Salmonella minnesota R595 (Re) (ultra pure, liquid), and Macrophage-Activating Lipopeptide-2 (MALP-2, S and R form) were obtained from Alexis, Germany. Highly purified lipoteichoic acid was the kind gift of T. Hartung. Unmethylated DNA oligonucleotide 5′-TCCATGACGTTCCTGATGCT-3′ was synthesized by Integrated DNA Technologies (Coralville, Iowa). dsRNA was obtained from Amersham Pharmacia Biotech. Resiquimod was obtained from 3M Corporation. Pam₂CSK₄, and Pam₃CSK₄ were obtained from EMC microcollections (Tuibingen, Germany). Zymosan A was obtained from Sigma. All were used at the stated concentrations. Recombinant soluble CD14 was purchased from CellSciences (Canton, Mass.). rMuIFN-β and an IFN-α ELISA kit were obtained from R&D systems. BfuA-I, used in sequence analysis, was obtained from New England Biolabs. RT-PCR was performed by using ThermoScript RT-PCR systems from Invitrogen. Total RNA was isolated from cells and type-I interferon induction were analyzed by RT-PCR for 28-30 cycles at 94° C. for 30 s, 58° C. for 30 s, and 68° C. for 40 s. The IFN-β, ISG15, IFIT1, IL-12β, HRPT cDNAs were amplified with the following primers: 5′-TTCCTGCTGTGCTTCTCCAC-3′ and 5′-AAGGTACCTTTGCACCCTCC-3′ for IFN-β, 5′-TGGGACCTAAAGGTGAAGATGCTG-3′ and 5′-TGCTTGATCACTGTGCACTGGG-3′ for ISG15, 5′-TCACTTCACATGGAAGCTGCTATTTG-3′ and 5′-CCATGGCTTGTTTATAATTTCCTCCTC-3′ for ]FIT1, 5′-CGGGTCTGGTTTGATGATGTCC-3′ and 5′-GACCCTGACCATCACTGTCAAAGAG-3′ for L-12β, 5′-GGACAGGACTGAAAGACTTGCTCG-3′ and 5′-TCCAACAAAGTCTGGCCTGTATCC-3′ for HRPT. JumpStart RED AccuTaq LA DNA Polymerases was obtained from Sigma. Antibody against IRF-3 (for detection of phosphodimer in the native gel) was obtained from Santa Cruz Biotechnology, antibodies against phosphorylated IκB and ERK1/2 (p42/p44) were from Cell Signaling (Beverly, Mass.), antibody against IRF3, β-Tubulin was from Zymed (South San Francisco, Calif.) and Pharmingen (San Diego, Calif.) respectively.

Biological assays. Type I IFN activity was measured with reference to a recombinant mouse IFN-β standard using an L-929 cell line transfected with an interferon-sensitive luciferase construct. TNF activity produced by peritoneal macrophages was determined with reference to a recombinant mouse TNF standard using the L-929 cells cytolytic assay. To measure the lytic effect of VSV on TG-elicited peritoneal macrophages, cells were plated at a density of 10⁵ per well in 96-well plates and each well was inoculated with virus, which was separately titred by plaque-forming assay on L-929 cell monolayers. Cells were stained with MTT to assess viability after the stated time interval.

Immunoblotting. Peritoneal macrophages, untreated or treated with smooth LPS or Lipid A for indicated times, were lysed in lysis buffer (0.5% Triton X-100, 20 mM HEPES, pH 7.4, 150 mM NaCl, 12.5 mM ³-glycerophosphate, 1.5 mM MgCl₂, 10 mM NaF, 2 mM dithiothreitol, 1 mM sodium orthovanadate, 2 mM EGTA, 20 μM aprotinin, 1 mM phenyl-methylsulfonyl fluoride). Cell extracts were separated by SDS-PAGE, transferred to Immobilon-P membranes (Millipore), and analyzed by immunoblotting using antibody against phospho-ERK, phospho-IκB, IRF3, and β-Tubulin. Protein analysis of IRF-3 phosphodimer formation was performed as described previously. Poltorak et al., TGGGACCTAAAGGTGAAGATGCTG-Science 282: 2085-2088, 1998.

When ranges are used herein for physical properties, such as molecular weight, or chemical properties, such as chemical formulae, all combinations and subcombinations of ranges and specific embodiments therein are intended to be included.

The disclosures of each patent, patent application and publication cited or described in this document are hereby incorporated herein by reference, in their entirety.

Those skilled in the art will appreciate that numerous changes and modifications can be made to the embodiments of the invention and that such changes and modifications can be made without departing from the spirit of the invention. It is, therefore, intended that the appended claims cover all such equivalent variations as fall within the true spirit and scope of the invention.

What is claimed:

1. A method for treating rhabdovirus infection in a mammalian subject suspected of having an infection comprising administering to the subject a modulator of Toll-like receptor 4-signaling activity via CD14 in an amount effective to reduce or eliminate the rhabdovirus infection or to prevent its occurrence or recurrence.

2. The method of claim 1 wherein the modulator is an antagonist of Toll-like receptor 4-signaling activity via CD14.

3. The method of claim 1 wherein the modulator is an inhibitor of CD14 activity or Toll-like receptor 4-signaling activity.

4. The method of claim 3 wherein the inhibitor is interfering RNA, short hairpin RNA, ribozyme, or antisense oligonucleotide to CD14 or TLR-4.

5. The method of claim 3 wherein the inhibitor is a monoclonal antibody, a polyclonal antibody, a peptide, peptidomimetic, or a small chemical inhibitor to CD14 or TLR-4.

6. The method of claim 5 wherein the inhibitor is an antibody to CD14.

7. The method of claim 5 wherein the inhibitor is an antibody to TLR-4.

8. The method of claim 5 wherein the rhabdovirus is rabies virus or vesicular stomatitis virus.

9. A method for treating an autoimmune disease in a mammalian subject comprising administering to the mammalian subject a modulator of Toll-like receptor 4-signaling activity via CD14 in an amount effective to reduce or eliminate the autoimmune disease or to prevent its occurrence or recurrence.

10. The method of claim 9 wherein the modulator is an antagonist of Toll-like receptor 4-signaling activity via CD14.

11. The method of claim 10 wherein the modulator is an inhibitor of CD14 activity or Toll-like receptor 4-signaling activity.

12. The method of claim 11 wherein the inhibitor is a monoclonal antibody, a polyclonal antibody, a peptide, peptidomimetic, or a small chemical inhibitor to CD14 or TLR-4.

13. The method of claim 11 wherein the inhibitor is an antibody to CD14.

14. The method of claim 11 wherein the inhibitor is an antibody to TLR-4.

15. A method for treating inflammation in a mammalian subject comprising administering to the mammalian subject a modulator of Toll-like receptor 4-signaling activity via CD14 in an amount effective to reduce or eliminate inflammation or to prevent its occurrence or recurrence.

16. The method of claim 15 wherein the modulator is an antagonist of Toll-like receptor 4-signaling activity via CD14.

17. The method of claim 16 wherein the modulator is an inhibitor of CD14 activity or Toll-like receptor 4-signaling activity.

18. The method of claim 17 wherein the inhibitor is a monoclonal antibody, a polyclonal antibody, a peptide, peptidomimetic, or a small chemical inhibitor to CD14 or TLR-4.

19. The method of claim 17 wherein the inhibitor is an antibody to CD14.

20. The method of claim 17 wherein the inhibitor is an antibody to TLR-4.

21. A method for identifying a modulator of signaling in cells via a toll-like receptor 4 pathway comprising:
contacting a test compound with a cell-based assay system comprising a cell expressing toll-like receptor 4 capable of signaling responsiveness to a ligand;
providing CD14 and the ligand to the assay system in an amount selected to be effective to activate toll-like receptor 4 signaling; and
detecting an effect of the test compound on toll-like receptor 4 signaling in the assay system, effectiveness of the test compound in the assay being indicative of the modulation.

22. The method of claim 21 further comprising coexpressing CD14 and toll-like receptor 4 in the cell.

23. The method of claim 21, further comprising providing toll-like receptor 4 to the assay system, and detecting an effect of the test compound on CD14/toll-like receptor 4 signaling in the assay system, effectiveness of the test compound in the assay being indicative of the modulation.

24. The method of claim 21 wherein the ligand is an endogenous ligand or an exogenous ligand.

25. The method of claim 24 wherein the exogenous ligand is lipopolysaccharide, lipid A, di-acylated lipopeptide, tri-acylated lipopeptide, S-MALP-2, R-MALP-2, bacterial lipopeptide, Pam2CSK4, lipoteichoic acid, or zymosan A.

26. The method of claim 24 wherein the exogenous ligand is rough lipopolysaccharide, smooth lipopolysaccharide, or lipid A from Salmonella minnesota.

27. The method of claim 26 wherein the detecting step further comprises measuring an effect on tumor necrosis factor production in the cell wherein TNF production is altered in response to rough lipopolysaccharide, but not in response to smooth lipopolysaccharide or lipid A from Salmonella minnesota.

28. The method of claim 21 wherein the endogenous ligand is a lipid.

29. The method of claim 21 wherein the detecting step further comprises effecting reduced binding of ligand to CD14 by the compound.

30. The method of claim 21 wherein the detecting step further comprises effecting reduced binding of CD14 to toll-like receptor 4 by the compound.

31. The method of claim 21 wherein the detecting step further comprises effecting enhanced binding of ligand to CD14 by the compound.

32. The method of claim 21 wherein the detecting step further comprises effecting enhanced binding of CD14 to toll-like receptor 4 by the compound.

33. The method of claim 30 wherein the compound is an antagonist of toll-like receptor 4 pathway signaling.

34. The method of claim 32 wherein the compound is an agonist of toll-like receptor 4 pathway signaling.

35. The method of claim 33 wherein the detecting step further comprises measuring a decrease in tumor necrosis factor in the cell assay.

36. The method of claim 34 wherein the detecting step further comprises measuring an increase in tumor necrosis factor in the cell assay.

37. The method of claim 32 wherein the cell assay further comprises a macrophage cell.

38. The method of claim 21 wherein the detecting step further comprises measuring labeled CD14 binding to ligand or labeled CD14 binding to toll-like receptor 4.

39. The method of claim 38 wherein the label is radiolabel or fluorescent label.

40. The method of claim 21 wherein the cell expresses TRAM-Trif capable of signaling responsiveness to the ligand;
providing CD14 and the ligand to the assay system in an amount selected to be effective to activate TRAM-Trif signaling; and
detecting an effect of the test compound on TRAM-Trif signaling in the assay system, effectiveness of the test compound in the assay being indicative of the modulation.

41. The method of claim 40 further comprising coexpressing CD14, toll-like receptor 4, and TRAM-Trif in the cell.

42. The method of claim 40, further comprising providing toll-like receptor 4 to the assay system, and detecting an effect of the test compound on CD14/toll-like receptor 4/TRAM-Trif signaling in the assay system, effectiveness of the test compound in the assay being indicative of the modulation.

43. The method of claim 40 wherein the detecting step further comprises effecting reduced binding of ligand to toll-like receptor 4 by the compound.

44. The method of claim 40 wherein the detecting step further comprises effecting reduced binding of toll-like receptor 4 to TRAM-Trif by the compound.

45. The method of claim 40 wherein the detecting step further comprises effecting enhanced binding of ligand to CD14 by the compound.

46. The method of claim 40 wherein the detecting step further comprises effecting enhanced binding of toll-like receptor 4 to TRAM-Trif by the compound.

47. The method of claim 40 wherein the compound is an agonist of TRAM-Trif pathway signaling.

48. The method of claim 40 wherein the compound is an antagonist of TRAM-Trif pathway signaling.

49. The method of claim 40 wherein the ligand is an endogenous ligand or an exogenous ligand.

50. The method of claim 49 wherein the exogenous ligand is lipopolysaccharide.

51. The method of claim 49 wherein the endogenous ligand is lipid.

52. The method of claim 47 wherein the detecting step further comprises measuring an increase in phosphorylation of IRF-3 in the cell assay.

53. The method of claim 48 wherein the detecting step further comprises measuring a decrease in phosphorylation of IRF-3 in the cell assay.

54. The method of claim 47 wherein the detecting step further comprises measuring an increase in interferon-β in the cell assay.

55. The method of claim 48 wherein the detecting step further comprises measuring a decrease in interferon-β in the cell assay.

56. The method of claim 47 wherein the detecting step further comprises measuring a decreased susceptibility to viral infectivity in the cell assay.

57. The method of claim 48 wherein the detecting step further comprises measuring an increased susceptibility to viral infectivity in the cell assay.

58. The method of claim 40 wherein the cell assay further comprises a macrophage cell.

59. The method of claim 40 wherein the detecting step further comprises measuring labeled CD14 binding to ligand or labeled CD14 binding to TLR4 or TRAM-Trif.

60. The method of claim 59 wherein the label is radiolabel or fluorescent label.

61. A method for screening for a compound to treat an infectious disease comprising:
contacting a test compound with a cell-based assay system comprising a cell expressing toll-like receptor 4 capable of signaling responsiveness to a ligand;
providing CD14 and the ligand to the assay system in an amount selected to be effective to activate toll-like receptor 4 signaling; and
detecting an effect of the test compound on toll-like receptor 4 signaling in the assay system, effectiveness of the test compound in the assay being indicative of the modulation of the infectious disease.

62. The method of claim 61 wherein the cell expresses TRAM-Trif capable of signaling responsiveness to the ligand;
providing CD14 and the ligand to the assay system in an amount selected to be effective to activate TRAM-Trif signaling; and
detecting an effect of the test compound on TRAM-Trif signaling in the assay system, effectiveness of the test compound in the assay being indicative of the modulation of the infectious disease.

63. The method of claim 61 wherein the compound is an antagonist of toll-like receptor 4 signaling to the ligand.

64. The method of claim 62 wherein the compound is an antagonist of toll-like receptor 4 signaling to the ligand.

65. The method of claim 61 wherein the infectious disease is a bacterial or viral disease.

66. The method of claim 65 wherein the infectious disease is rhabdovirus infection, rabies virus infection, vesicular stomatitis virus infection, HIV infection, AIDS, cytomegalovirus infection, or Staphylococcus aureus infection.

67. The method of claim 66 wherein the compound is an inhibitor of rhabdovirus G glycoprotein interaction with CD14.

68. A method for screening for a compound to treat an autoimmune disease comprising:
contacting a test compound with a cell-based assay system comprising a cell expressing toll-like receptor 4 capable of signaling responsiveness to a ligand;
providing CD14 and the ligand to the assay system in an amount selected to be effective to activate toll-like receptor 4 signaling; and
detecting an effect of the test compound on toll-like receptor 4 signaling in the assay system, effectiveness of the test compound in the assay being indicative of the modulation of the autoimmune disease.

69. The method of claim 68 wherein the cell expresses TRAM-Trif capable of signaling responsiveness to the ligand;
providing CD14 and the ligand to the assay system in an amount selected to be effective to activate TRAM-Trif signaling; and
detecting an effect of the test compound on TRAM-Trif signaling in the assay system, effectiveness of the test compound in the assay being indicative of the modulation of the autoimmune disease.

70. The method of claim 68 wherein the compound is an antagonist of toll-like receptor 4 signaling to the ligand.

71. The method of claim 68 wherein the autoimmune disease is insulin-dependent diabetes mellitus, multiple sclerosis, experimental autoimmune encephalomyelitis, rheumatoid arthritis, experimental autoimmune arthritis, myasthenia gravis, thyroiditis, an experimental form of uveoretinitis, Hashimoto's thyroiditis, primary myxoedema, thyrotoxicosis, pernicious anaemia, autoimmune atrophic gastritis, Addison's disease, premature menopause, male infertility, juvenile diabetes, Goodpasture's syndrome, pemphigus vulgaris, pemphigoid, sympathetic ophthalmia, phacogenic uveitis, autoimmune haemolytic anaemia, idiopathic leucopenia, primary biliary cirrhosis, active chronic hepatitis Hbₛ-ve, cryptogenic cirrhosis, ulcerative colitis, Sjogren's syndrome, scleroderma, Wegener's granulomatosis, poly/dermatomyositis, discoid LE or systemic lupus erythematosus.

72. A method for screening for a compound to treat inflammation comprising:
contacting a test compound with a cell-based assay system comprising a cell expressing toll-like receptor 4 capable of signaling responsiveness to a ligand;
providing CD14 and the ligand to the assay system in an amount selected to be effective to activate toll-like receptor 4 signaling; and
detecting an effect of the test compound on toll-like receptor 4 signaling in the assay system, effectiveness of the test compound in the assay being indicative of the modulation of the autoimmune disease.

73. The method of claim 72 wherein the cell expresses TRAM-Trif capable of signaling responsiveness to the ligand;
providing CD14 and the ligand to the assay system in an amount selected to be effective to activate TRAM-Trif signaling; and
detecting an effect of the test compound on TRAM-Trif signaling in the assay system, effectiveness of the test compound in the assay being indicative of the modulation of the autoimmune disease.

74. The method of claim 72 wherein the compound is an antagonist of toll-like receptor 4 signaling to the ligand.

75. A transgenic non-human animal comprising a heterologous nucleic acid wherein the nucleic acid comprises a loss-of-function allele of a CD14 gene, and the animal exhibits a phenotype, relative to a wild-type phenotype, comprising a characteristic of inhibition of macrophage activation, susceptibility to viral or bacterial infection, a decrease in TNF-α production, or a combination of any two or more thereof.

76. The transgenic non-human animal of claim 75 wherein the phenotype of the CD14 mutant animal is characteristic of decreased phosphorylation and dimerization of IRF-3 upon induction by lipopolysaccharide, non-responsive INF-β production upon induction by lipopolysaccharide, or macrophage hypersensitivity to cytolysis induced by vesicular stomatitis virus or rabies virus.

77. The transgenic non-human animal of claim 75 wherein the loss-of-function allele in the CD14 gene is a premature stop codon at Q284X.

78. The transgenic non-human animal of claim 75 wherein the animal is a mouse or a rat.

79. A cell or cell line derived from a transgenic non-human animal according to claim 75.

80. An in vitro method of screening for a modulator of a Toll-like receptor 4-or TRAM-Trif-signaling activity, the method comprising: contacting a cell or cell line according to claim 79 with a test compound; and detecting an increase or a decrease in the amount of TNF-α production, susceptibility to viral or bacterial infection, or a Toll-like receptor 4- or TRAM-Trif-induced macrophage activating activity, thereby identifying the test compound as a modulator of the Toll-like receptor 4- or TRAM-Trif-induced macrophage activating activity.

81. An in vivo method of screening for a modulator of a Toll-like receptor 4- or TRAM-Trif-signaling activity, the method comprising: contacting a cell or cell line according to claim 79 with a test compound; and detecting an increase or a decrease in the amount of TNF-α production, susceptibility to viral or bacterial infection, or a Toll-like receptor 4- or TRAM-Trif-induced macrophage activating activity, thereby identifying the test compound as a modulator of a Toll-like receptor 4- or TRAM-Trif-induced macrophage activating activity.
